(19) United States
(12) Patent Application Publication
(10) Pub. No.: US 2026/0140128 A1
(43) Pub. Date: May 21, 2026

(54) SYSTEMS AND METHODS FOR SAMPLE HANDLING

(71) Applicant: BECTON, DICKINSON AND COMPANY, Franklin Lakes, NJ (US)

(72) Inventors: Brent Ronald Pohl, Timonium, MD (US); Dwight Livingston, Fallston, MD (US); Edward M. Skevington, Stewartstown, PA (US); Robert Edward Armstrong, Hunt Valley, MD (US); Anand Jasani, Parsippany, NJ (US); Timothy Roy Hansen, Spring Grove, PA (US); Christopher B. Godschall, Ellicott City, MD (US); Franciscus Hermannus Feijen, Leeuwarden (NL); Joshua Herr, Phoenix, MD (US); Sindhushree Raghunandan, Baltimore, MD (US); Stephen Robert LaChance, Cockeysville, MD (US); Paul Rushton, London (GB); David J. Turner, York, PA (US); Mark A. Renoll, York, PA (US); Matthew Swecker, Raleigh, NC (US); Luke Peterken, Abingdon, MD (US); Rita Matta, Morristown, NJ (US)

(73) Assignee: BECTON, DICKINSON AND COMPANY, Franklin Lakes, NJ (US)

(21) Appl. No.: 18/995,305

(22) PCT Filed: Jul. 18, 2023
(86) PCT No.: PCT/US2023/028036

§ 371 Date: Jan. 16, 2025

Related U.S. Application Data
(63) Continuation-in-part of application No. 29/890,968, filed on Apr. 28, 2023, now Pat. No. D1094770.
(60) Provisional application No. 63/390,535, filed on Jul. 19, 2022.
(60) Provisional application No. 63/432,533, filed on Dec. 14, 2022.

(51) Int. Cl. G01N 35/00; C12M 1/00; C12M 1/26; C12M 1/34; C12M 1/36; G01F 23/292; G01N 35/04

(52) U.S. Cl. CPC G01N 35/0099; C12M 33/00; C12M 41/14; C12M 41/36; C12M 41/48; G01F 23/2921; G01N 35/00732; G01N 35/04; G01N 2035/00792; G01N 2035/0091; G01N 2035/046

(57) ABSTRACT

Systems and methods for receiving batches of sample containers (e.g., blood culture bottles), sorting the sample containers, and discarding negative sample containers in a safe, secure, and consistent manner are disclosed. Additionally, systems and methods to accurately and precisely determine the amount of sample inoculated into a container are disclosed. For example, an apparatus determines the amount of blood inoculated into a blood culture bottle by weighing and/or obtaining an image of the blood culture bottle inoculated with blood sample. Such an approach facilitates automation, as a user does not need to visually inspect each bottle.

### CROSS-REFERENCE TO RELATED APPLICATIONS

This application claims priority from U.S. Provisional Application No. 63/390,535, filed Jul. 19, 2022, U.S. Provisional Application No. 63/432,533, filed Dec. 14, 2022, and U.S. Design Application No. Ser. No. 29/890,968, filed Apr. 28, 2023, all of which are incorporated herein by reference. This application is also related to International Application No. PCT/US 2022/019424, filed Mar. 9, 2022, which claims priority from U.S. Provisional Application No. 63/159,269, filed Mar. 10, 2021, both of which are incorporated herein by reference.

### TECHNICAL FIELD

The present disclosure relates to systems and methods for sample handling. For example, in some implementations, a system may be configured to automatically process a plurality of blood culture bottles.

### BACKGROUND OF THE INVENTION

The presence of biologically active agents, such as bacteria in a patient's body fluid, especially blood, is generally determined using culture bottles, such as the BD BACTEC™ culture bottles, which are manufactured and sold by Becton, Dickinson and Company. The culture bottles may contain a blood culture media, such as the BD BACTEC™ Peds Plus™ medium, BD BACTEC™ Plus Aerobic medium, BD BACTEC™ Plus Anaerobic medium, BD BACTEC™ Lytic Anaerobic medium, BD BACTEC™ Standard Aerobic medium, BD BACTEC™ Standard Anaerobic medium, BD BACTEC™ Myco medium, BD BACTEC™ Mycosis medium, BD BACTEC™ Aerobic Platelet medium, or BD BACTEC™ Anaerobic Platelet medium, all of which are manufactured and sold by Becton, Dickinson and Company. To test for the presence of biologically active agents, a small quantity of blood or other bodily fluid is injected through an enclosing rubber septum into a sterile culture bottle containing a culture medium, and the bottle is then incubated at about 35-37° C. (e.g., 36.5° C.) and monitored for microorganism growth. Microbial growth may be detected by a change in the blood culture over time. Parameters, such as the concentration of carbon dioxide or oxygen in the culture bottle headspace or a change in pH, may be monitored for changes over time that are indicative of microbial growth.

Since it is of utmost importance to learn if a patient has a bacterial infection, hospitals and laboratories have automated apparatus that may process many blood culture bottles simultaneously. One example of such an apparatus is the BD BACTEC™ FX blood culture system, which is manufactured and sold by Becton, Dickinson and Company. U.S. Pat. No. 5,817,508, entitled “Blood Culture Apparatus having an Auto-Unloading and Sorting Device,” describes a prior art blood culture apparatus, and is incorporated herein by reference. Additional descriptions of blood culture apparatus are provided in U.S. Pat. No. 5,516,692, entitled “Compact Blood Culture Apparatus,” and U.S. Pat. No. 5,498,543, entitled “Sub-Compact Blood Culture Apparatus,” both of which are incorporated herein by reference.

It is critical to ensure that the presence or absence of a blood stream infection (BSI) is correctly determined. Patients and their caregivers are placed at risk if a BSI goes undetected. It is well known that overfilling a blood culture bottle with the blood sample may lead to false positives. It is also well known that underfilling blood culture bottles with the blood sample may lead to false negatives. This is because the sample removed from the patient has a certain, but unknown, concentration of bacteria (if bacteria is at all present). Therefore, in the case of underfill, a lower bacteria count is present in the blood culture bottle at time zero than if the culture bottle had been filled with the target sample amount. It follows then that, in the case of overfill, a higher bacteria count is present in the blood culture bottle at time zero than if the culture bottle had been filled with the target sample (e.g., blood) amount. If a bottle is overfilled or underfilled, algorithms may be applied to the measured changes in carbon dioxide or oxygen concentration or pH to adjust for underfill or overfill. If the underfill or overfill exceeds a certain specification, the blood culture bottles are discarded. This is described in U.S. Pat. No. 9,365,814, entitled “System and Method for Determining Fill Volume in a Container,” which is incorporated herein by reference.

Therefore, when processing blood culture bottles in a laboratory environment that is processing a large number of blood culture bottles, there is a need to be able to monitor the fill condition of each bottle accurately. Other information about the blood culture, such as the label information, may also need to be collected. Consequently, methods and apparatus that may accurately obtain fill information and label information from a blood culture bottle continue to be sought.

Additionally, even when using existing automated apparatus, it can still be laborious and time consuming for operators to process blood culture bottles. For example, operators may need to individually load and unload blood culture bottles from an automated blood culture apparatus. Operators may also need to manually separate blood culture bottles that tested positive from blood culture bottles that tested negative, discard the negative blood culture bottles into waste receptacles, and prepare the positive blood culture bottles for additional microbiological workup. Such lab environments fail to realize maximum efficiency due to delays in manual handling and processing of the samples. These delays can cause unnecessary delays in diagnosing and treating patients. Moreover, operator errors (e.g., human errors) may result when the operators are tired, distracted, or otherwise unfocused and can lead to processing errors, which may result in an incorrect diagnosis, a late diagnosis, and lost or ruined samples. Such errors can potentially adversely affect patients and waste resources.

Thus, there is also a need in the art for a lab automation system that is capable of receiving batches of blood culture bottles, separating positive and negative blood culture bottles, and discarding negative blood culture bottles in a safe, secure, and consistent manner at all times of the day with minimal intervention from a small number of lab operators.

### BRIEF SUMMARY OF THE INVENTION

Described herein are systems and methods for receiving batches of sample containers (e.g., blood culture bottles), sorting the sample containers, and discarding negative sample containers in a safe, secure, and consistent manner. Additionally, systems and methods are described herein to accurately and precisely determine the amount of sample inoculated into a container. For example, an apparatus is described herein that determines the amount of blood inoculated into a blood culture bottle by weighing and/or obtaining an image of the blood culture bottle inoculated with blood sample. Such an approach facilitates automation, as a user does not need to visually inspect each bottle.

One aspect of the present disclosure relates to a sample handling module comprising a user interface subsystem, an imaging subsystem, a waste management subsystem, and a robotic subsystem. The user interface subsystem may be configured to receive a plurality of untested sample containers and output a plurality of sample containers that tested positive for microbial growth. The imaging subsystem may be configured to scan sample containers for label information. The waste management subsystem may be configured to receive a plurality of sample containers that tested negative for microbial growth. The robotic subsystem may be configured to (a) transfer each of the plurality of untested sample containers from the user interface subsystem to the imaging subsystem for scanning, (b) transfer each of the plurality of untested sample containers from the imaging subsystem to an incubation module configured to incubate each of the plurality of untested sample containers and measure microbial growth, (c) transfer each of the plurality of positive sample containers from the incubation module to the imaging subsystem for scanning, (d) transfer each of the plurality of positive sample containers from the imaging subsystem to the user interface subsystem for output, (e) transfer each of the plurality of negative sample containers from the incubation module to the imaging subsystem for scanning, and (f) transfer each of the plurality of negative sample containers from the imaging subsystem to the waste management subsystem for disposal.

In some implementations, the user interface subsystem comprises one or more compartments, each of which is configured to receive individual untested sample containers or a rack of untested sample containers. In some implementations, the user interface subsystem comprises one or more compartments, each of which is configured to output individual positive sample containers or a rack of positive sample containers. In some implementations, the user interface subsystem comprises one or more compartments, each of which is configured to (a) receive individual untested sample containers or a rack of untested sample containers and (b) output individual positive sample containers or a rack of positive sample containers.

In some implementations, the user interface subsystem comprises one or more compartments, each of which has a liner with one or more sections, wherein each of the one or more sections comprises (a) a plurality of receptacles configured to accept sample containers directly and (b) a pair of recesses configured to accept a rack of sample containers. In some implementations, the user interface subsystem further comprises a sliding door configured to prevent a user from loading one or more untested sample containers into at least one of the compartments while the at least one compartment is being loaded with one or more positive sample containers by the robotic subsystem. In some implementations, the user interface subsystem further comprises one or more output chutes, each of which is configured to output individual sample containers. In some implementations, the user interface subsystem further comprises a display having a graphical user interface (GUI) through which the one or more compartments and the one or more output chutes may be selected for the output of positive sample containers.

In some implementations in which the user interface subsystem comprises one or more compartments, the user interface subsystem may further comprise one or more illumination lights, each of which is configured to change color based on the type of sample containers positioned within at least one of the compartments. In some implementations, each one of the illumination lights is configured to (a) change to a first color when at least one of the compartments contains one or more untested sample containers and (b) change to a second color when the at least one compartment contains one or more positive sample containers. In some implementations, the one or more illumination lights are positioned above the liner of at least one compartment.

In some implementations in which the user interface subsystem comprises one or more compartments, at least one of the compartments may comprise a scale and the sample handling module may further comprise one or more processors configured to determine whether the untested sample containers are overfilled or underfilled based weight measurements received from the scale. For example, in some implementations, the one or more processors may be configured to (a) receive from the scale a first measured weight of a plurality of untested sample containers in the at least one compartment, (b) control the robotic subsystem to transfer one of the untested sample containers from the at least one compartment to the imaging subsystem, (c) receive from the scale a second measured weight of the untested sample containers in the at least one compartment without the one untested sample container, (d) determine a difference between the first and second measured weights, and (e) store the difference as a weight of the one untested sample container in memory.

In some implementations, the user interface subsystem further comprises (a) a reader configured to scan identifiers on sample containers and (b) a display configured to provide information for sample containers scanned by the reader. In some implementations, the user interface subsystem further comprises a reader configured to scan an identifier on a user identification card to initiate an automatic login or automatically adjust one or more system settings.

In some implementations, each of the plurality of untested sample containers is received at the user interface subsystem in an upright position, and each of the plurality of untested sample containers is transferred from the imaging subsystem to the incubation module in a horizontal position. In some implementations, each of the plurality of positive sample containers is transferred from the incubation module to the imaging subsystem in a horizontal position, and each of the plurality of positive sample containers is transferred from the imaging subsystem to the user interface subsystem in an upright position. In some implementations, the imaging subsystem comprises (a) a camera for scanning the sample containers or capturing one or more images of the sample containers, (b) one or more light sources for illuminating the sample containers, (c) a chute configured to reorient sample containers from an upright position to a horizontal position, and (d) a flip station configured to reorient sample containers from a horizontal position to an upright position.

In some implementations, the imaging subsystem is further configured to capture one or more images of the sample containers, and the sample handling module further comprises one or more processors configured to determine whether the sample containers are overfilled or underfilled based on the captured images. For example, in some implementations, the one or more processors may be configured to (a) identify a position of a fill line on a sample container in the one or more images, (b) identifying a position of a reference surface on the sample container in the one or more images, (c) determine a distance between the fill line and the reference surface, (d) determine a correction factor based on a comparison between the determined distance and a predetermined distance, (e) adjust a predetermined tare weight with the correction factor, and (f) determine whether the sample container is overfilled or underfilled based on a comparison between the adjusted predetermined tare weight and a measured weight of the sample container obtained with a scale. In some implementations, the scale is coupled to the chute of the imaging subsystem, and wherein the measured weight is obtained while the sample container is positioned in the chute.

In some implementations, the waste management subsystem comprises a waste receptacle and one or more chutes through which the robotic subsystem can transfer the plurality of negative sample containers into the waste receptacle. In some implementations, the waste management subsystem comprises a load cell configured to (a) detect whether a waste receptacle if full, (b) detect whether a waste receptacle is positioned on a base, or (c) detect the addition of a sample container to a waste receptacle.

Another aspect of the present disclosure relates to an automated system comprising a sample handling module and an incubation module. The sample handling module may comprise a user interface subsystem, an imaging subsystem, a waste management subsystem, and a robotic subsystem. The user interface subsystem may be configured to receive a plurality of untested sample containers and output a plurality of sample containers that tested positive for microbial growth. The imaging subsystem may be configured to scan sample containers for label information. The waste management subsystem may be configured to receive a plurality of sample containers that tested negative for microbial growth. The robotic subsystem may be configured to (a) transfer each of the plurality of untested sample containers from the user interface subsystem to the imaging subsystem for scanning, (b) transfer each of the plurality of untested sample containers from the imaging subsystem to an incubation module configured to incubate each of the plurality of untested sample containers and measure microbial growth, (c) transfer each of the plurality of positive sample containers from the incubation module to the imaging subsystem for scanning, (d) transfer each of the plurality of positive sample containers from the imaging subsystem to the user interface subsystem for output, (e) transfer each of the plurality of negative sample containers from the incubation module to the imaging subsystem for scanning, and (f) transfer each of the plurality of negative sample containers from the imaging subsystem to the waste management subsystem for disposal. The incubation module may be configured to incubate each of the plurality of untested sample containers and measure microbial growth.

In some implementations, the incubation module comprises a motor and a drum having a plurality of receptacles, wherein each receptacle is configured to receive a sample container in a horizontal position, and wherein the motor is configured to rotate the drum. In some implementations, the robotic subsystem is further configured to distribute and redistribute sample containers around a circumference of the drum to balance a load of the drum. In some implementations, the robotic subsystem is further configured to redistribute sample containers to a specific area of the drum that can be viewed entirely when a door to the incubation module is open.

Yet another aspect of the present disclosure relates to a robotic system comprising (a) a gripper assembly configured to grab and release sample containers, (b) an r-axis robot configured to move the gripper assembly forwards and backwards, (c) a theta-axis robot configured to simultaneously rotate the r-axis robot and the gripper assembly, and (d) a z-axis robot configured to simultaneously move the theta-axis robot, the r-axis robot, and the gripper assembly upwards and downwards.

In some implementations, the gripper assembly comprises a motor and two grippers, wherein the motor is configured to move the two grippers closer together to grasp a sample container and to move the two grippers farther apart to release a sample container, and wherein each gripper comprises (a) a curved body configured to grasp a bottom end of a sample container in a horizontal position and (b) a curved recess in the curved body that is configured to grasp a neck of a sample container in an upright position. In some implementations, the curved body is configured to grasp a bottom end of a blood culture bottle in a horizontal position, and the curved recess is configured to grasp a neck of a blood culture bottle in an upright position.

In some implementations, the gripper assembly comprises a motor and two grippers, wherein the motor is configured to move the two grippers closer together to grasp a sample container and to move the two grippers farther apart to release a sample container, and wherein each gripper comprises (a) a plurality of fingers configured to grasp a bottom end of a sample container in a horizontal position and (b) a curved recess in a body of the gripper that is configured to grasp a neck of a sample container in an upright position. In some implementations, the fingers are configured to grasp a bottom end of a blood culture bottle in a horizontal position, and the curved recess is configured to grasp a neck of a blood culture bottle in an upright position.

In some implementations, the r-axis robot comprises (a) a first arm coupled to the theta-axis robot, (b) a second arm coupled to the gripper assembly, wherein the second arm is slidingly engaged with the first arm and configured to move forwards and backwards, (c) a plurality of idler pulleys, (d) a motor coupled to the first arm and positioned between at least two of the idler pulleys, (e) a drive pulley coupled to a shaft of the motor, (f) a belt contacting each of the idler pulleys and the drive pulley, and (g) a clamp coupled to the belt and the second arm. In some implementations, the r-axis robot further comprises a belt tensioner configured to apply tension to the belt. In some implementations, the belt tensioner comprises (a) an idler pulley contacting the belt, (b) an arm rotatably coupled to the idler pulley and a coupling, and (c) a screw configured to apply force to the arm when tightened, wherein the force from the screw causes (i) the arm to rotate about an axis that extends through the coupling and (ii) the idler pulley to apply additional tension to the belt.

In some implementations, the theta-axis robot comprises (a) a platform coupled to the z-axis robot, (b) an idler pully coupled to the r-axis robot, (c) a motor coupled to the platform, (d) a drive pulley coupled to a shaft of the motor, and (e) a belt contacting the idler pulley and the drive pulley, wherein rotation of the drive pully by the motor causes the idler pully, the r-axis robot, and the gripper assembly to simultaneously rotate.

In some implementations, the z-axis robot comprises a rail slidingly engaged with the theta-axis robot. In some implementations, the z-axis robot further comprises a counterweight system comprising (a) a counterweight, (b) one or more pulleys, and (c) at least one cable that contacts the one or more pulleys and is coupled to both the counterweight and the theta-axis robot.

Yet another aspect of the present disclosure relates to a robotic system comprising (a) a gripper assembly configured to grab and release sample containers, (b) an r-axis robot configured to move the gripper assembly forwards and backwards, (c) a z-axis robot configured to simultaneously move the r-axis robot and the gripper assembly upwards and downwards, and (d) a theta-axis robot configured to simultaneously rotate the z-axis robot, the r-axis robot and the gripper assembly.

Yet another aspect of the present disclosure relates to a gripper assembly comprising a motor, a first gripper, and a second gripper. The motor is configured to move the first and second grippers closer together to grasp a sample container and to move the first and second grippers farther apart to release a sample container. Each gripper comprises a first engagement feature configured to grasp a bottom end of a sample container in a horizontal position and a second engagement feature configured to grasp a neck of a sample container in an upright position.

In some implementations, the first engagement feature is a curved portion of a body of each gripper. In some implementations, the second engagement feature is a curved recess in the curved portion of the body of each gripper. In some implementations, the first engagement feature is a plurality of fingers. In some implementations, the second engagement feature is a curved recess in a body of each gripper.

In some implementations, the gripper assembly further comprises a non-contact sensor configured to verify movements of the robotic subsystem. In some implementations, the non-contact sensor is positioned between the two grippers. In some implementations, the non-contact sensor does not extend above or below the two grippers.

In some implementations, the gripper assembly further comprises (a) a first block comprising a first gear rack, wherein the first block is coupled to the first gripper, wherein the first block is slidingly coupled to a first rail, wherein as the first block slides along the first rail in a first direction, the first gripper moves farther away from the second gripper, and wherein as the first block slides along the first rail in a second direction, opposite the first direction, the first gripper moves closer to the second gripper; (b) a second block comprising a second gear rack, wherein the second block is coupled to the second gripper, wherein the second block is slidingly coupled to a second rail, wherein as the second block slides along the second rail in the second direction, the second gripper moves farther away from the first gripper, and wherein as the second block slides along the second rail in the first direction, the second gripper moves closer to the first gripper; and (c) a pinion gear coupled to a shaft of the motor, wherein the pinion gear is engaged with the first and second gear racks, wherein the motor is further configured to rotate the shaft, wherein rotation of the shaft causes the pinion gear to rotate, and wherein rotation of the pinion gear causes the first and second blocks to slide along first and second rails, respectively, in opposite directions.

In some implementations, the gripper assembly further comprises (a) a housing; (b) a base, wherein the motor is further configured to move the base forwards and backwards along a first axis that is perpendicular to a second axis along which the motor moves the first and second grippers, wherein the forward movement of the base causes the first and second grippers to move farther apart, and wherein the backward movement of the base causes the first and second grippers to move closer together; (c) a first plurality of members rotatably coupled to the first gripper and the housing; (d) a second plurality of members rotatably coupled to the second gripper and the housing; (e) a third member rotatably coupled to one of the first plurality of members and the base; and (f) a fourth member rotatably coupled to one of the second plurality of members and the base. In some implementations, the one of the first plurality of members, the one of the second plurality of members, the third member, and the fourth member are bent.

In some implementations, the gripper assembly further comprises (a) a flanged screw nut engaged with threads of a shaft of the motor, wherein the flanged screw nut extends through an opening of the base; (b) a spring plate slidingly engaged with the shaft of the motor, wherein the spring plate is coupled to the base; and (c) a spring, wherein a first end of the spring contacts the spring plate, and wherein a second end of the spring, opposite the first end, contacts the flanged screw nut. In some implementations, the motor is further configured to rotate the shaft, wherein rotation of the shaft causes the flanged screw nut to move forwards or backwards along the first axis, wherein the flanged screw nut pushes the base forward as the flanged screw nut moves forward, and wherein the flanged screw nut pushes against the spring as the flanged screw nut moves backward.

Yet another aspect of the present disclosure relates to a method comprising (a) obtaining, with one or more processors, a measured weight of a sample container, wherein the measured weight was measured with a scale, (b) selecting, with the one or more processors, a predetermined unfilled tare weight, wherein the predetermined unfilled tare weight is selected based on contents of the sample container or a lot or batch in which the sample container was manufactured, (c) comparing, with the one or more processors, the measured weight to the predetermined unfilled tare weight to compute a weight of a sample in the sample container, and (d) converting, with the one or more processors, the weight of the sample into a volume measurement based on a predetermined density value.

Yet another aspect of the present disclosure relates to a method comprising (a) obtaining, with one or more processors, a measured weight of a sample container, wherein the measured weight was measured with a scale, (b) obtaining, with the one or more processors, one or more images of the sample container, wherein the one or more images were captured with a camera, (c) identifying, with the one or more processors, a position of a fill line on the sample container in the one or more images, (d) identifying, with the one or more processors, a position of a reference surface on the sample container in the one or more images, (e) determining, with the one or more processors, a distance between the fill line and the reference surface, (f) determining, with the one or more processors, a correction factor based on a comparison between the determined distance and a predetermined distance, (g) adjusting, with the one or more processors, a predetermined tare weight with the correction factor, and (h) determining, with the one or more processors, whether the sample container is overfilled or underfilled based on a comparison between the adjusted predetermined tare weight and the measured weight.

Yet another aspect of the present disclosure relates to a method comprising (a) positioning a plurality of sample containers on scale, (b) measuring, with the scale, a weight of the plurality of sample containers, (c) removing one of the plurality of sample containers from the scale, (d) measuring, with the scale, a weight of the plurality of sample containers without the one sample container, (e) scanning, with a reader, an identifier on the one sample container while the weight of the plurality of sample containers without the one sample container is being measured, (f) determining, with one or more processors, a difference in weight between (i) the weight of the plurality of sample containers and (ii) the weight of the plurality of sample containers without the one sample container, and (g) storing, with the one or more processors, the difference in weight as a weight of the one sample container in memory.

### BRIEF DESCRIPTION OF DRAWINGS

FIG. 1A illustrates an automated system for processing a plurality of sample containers.

FIG. 1B illustrates a module of the automated system illustrated in FIG. 1A.

FIGS. 1C and 1D are close-up views of top and bottom portions, respectively of the module illustrated in FIG. 1B.

FIG. 1E illustrates the module of FIG. 1B with a cover.

FIG. 1F illustrates the module of FIG. 1B without a cover.

FIGS. 2A-2D are perspective views of the module of FIG. 1B without a housing. This exposes aspects of, for example, a user interface subsystem, an imaging subsystem, a waste management subsystem, and a robotic subsystem.

FIG. 2E is a close-up view of an electronics bay.

FIGS. 3A-3C are perspective views of a user interface subsystem, which includes compartments for receiving and outputting sample containers.

FIG. 3D is a close-up view of output chutes.

FIGS. 4A and 4B are perspective views of a liner, a rack, and sensors.

FIGS. 4C-4I illustrate different views of the rack of FIGS. 4A and 4B.

FIG. 5A is a perspective views of an imaging subsystem and output chutes.

FIGS. 5B and 5C are perspective views of the imaging subsystem illustrated in FIG. 5A.

FIG. 5D is a cross-sectional view of the imaging subsystem illustrated in FIG. 5A.

FIGS. 5E and 5F are perspective views of a chute.

FIG. 6A is a perspective view of a waste management subsystem.

FIG. 6B is an exploded view of a waste receptacle holder.

FIG. 7A is a perspective view of a robotic subsystem.

FIG. 7B is a perspective view of the robotic subsystem illustrated in FIG. 7A without a z-axis robot.

FIG. 7C is a perspective view of an r-axis robot and a gripper assembly of the robotic subsystem illustrated in FIG. 7A.

FIGS. 7D and 7E are perspective views of the gripper assembly and portions of the r-axis robot 730 illustrated in FIG. 7C.

FIGS. 7F-7H are perspective views of a theta-axis robot of the robotic subsystem illustrated in FIG. 7A.

FIGS. 8A-8E are perspective views of one or more racks.

FIGS. 9A and 9B are perspective views of a liner.

FIG. 9C is a cross-sectional view of the liner illustrated in FIGS. 9A and 9B.

FIG. 10 illustrates an imaging apparatus and a robotic gripper for placing a sample container vertically and removing the bottle horizontally.

FIG. 11 is a bottom perspective exploded view of the apparatus of FIG. 10.

FIGS. 12A-12D are perspective views of an imaging apparatus, wherein a gate is operated from beneath a platform to dispense a sample container when an image of the label on the sample container has been obtained.

FIG. 13 is a schematic perspective view of the imaging apparatus of FIG. 10 using a conical mirrored tray.

FIG. 14 is a schematic perspective view of the imaging apparatus of FIG. 13 using multiple cameras to obtain images of discrete portions of the label.

FIG. 15 is a schematic perspective view of the imaging apparatus of FIG. 13 without the trap door.

FIG. 16 is a schematic perspective view of the imaging apparatus of FIG. 10 without the trap door.

FIG. 17 is a schematic perspective view of the imaging apparatus of FIG. 16 with an alternative configuration for gripping the sample container.

FIG. 18 is a schematic perspective view of a chute for removing the sample container from the imaging apparatus after imaging.

FIG. 19 is a flow chart of a method described herein.

FIG. 20A is a perspective view of a robotic subsystem.

FIGS. 20B and 20C are perspective views of an r-axis robot and portions of a gripper assembly of the robotic subsystem illustrated in FIG. 20A.

FIGS. 20D and 20E are perspective views of the gripper assembly of the robotic subsystem illustrated in FIG. 20A.

FIG. 20F is a perspective view of a theta-axis robot of the robotic subsystem illustrated in FIG. 20A.

FIG. 20G is a cross-sectional view of portions of the theta-axis robot and the r-axis robot of the robotic subsystem illustrated in FIG. 20A.

FIG. 21A illustrates an automated system for processing a plurality of sample containers.

FIG. 21B is a perspective view of a robotic subsystem.

FIG. 21C is a perspective view of portions of the robotic subsystem illustrated in FIG. 21B grabbing a sample container.

FIG. 21D illustrates a top-down view of portions of the robotic subsystem illustrated in FIG. 21B grabbing a sample container.

FIGS. 21E and 21F are perspective views of the gripper assembly of the robotic subsystem illustrated in FIG. 21B.

FIGS. 22A and 22B are perspective views of a theta-axis robot.

FIG. 22C is a cross-sectional view of an idler pully.

FIG. 23A is a perspective view of a gripper assembly.

FIGS. 23B-23D are perspective views of the gripper assembly of FIG. 23A without portions of a housing.

FIG. 24A is a perspective view of a gripper assembly.

FIGS. 24B-24D are perspective views of the gripper assembly of FIG. 24A without portions of a housing.

FIG. 25A is a top perspective view of a gripper assembly.

FIG. 25B is a bottom perspective view of the gripper assembly of FIG. 25A.

FIG. 25C is a perspective view of the gripper assembly of FIG. 25A without a top portion of a housing.

FIG. 25D is a perspective view of the gripper assembly of FIG. 25A without a housing and some other stationary components.

FIGS. 25E-25G are top-down views of the gripper assembly of FIG. 25D.

FIGS. 25H and 25I are perspective views of a spring plate and a sensor.

FIG. 26A is a cross-sectional view of a chute with a scale.

FIGS. 26B and 26C are perspective views of the chute illustrated in FIG. 26A.

### DETAILED DESCRIPTION

Aspects of the present disclosure are described in detail with reference to the drawing figures wherein like reference numerals identify similar or identical elements. It is to be understood that the disclosed implementations are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

### System Overview

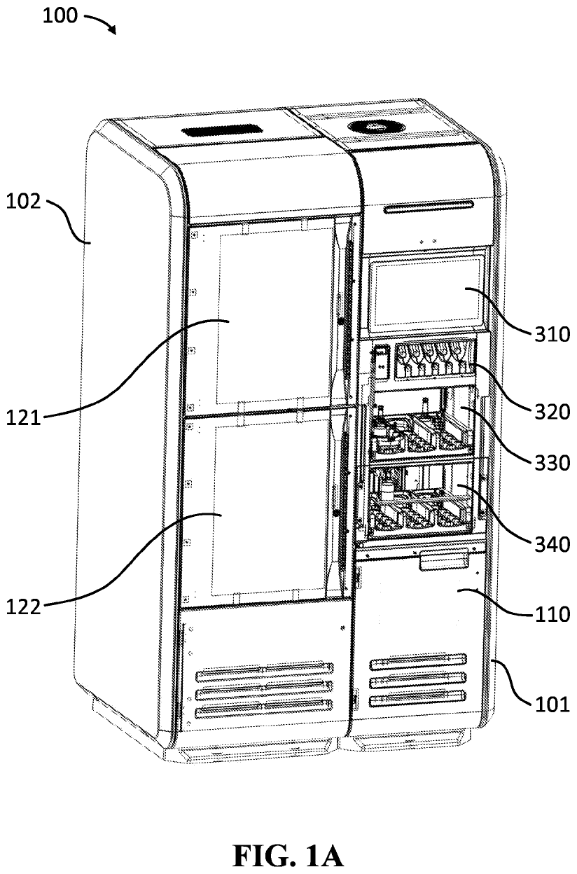

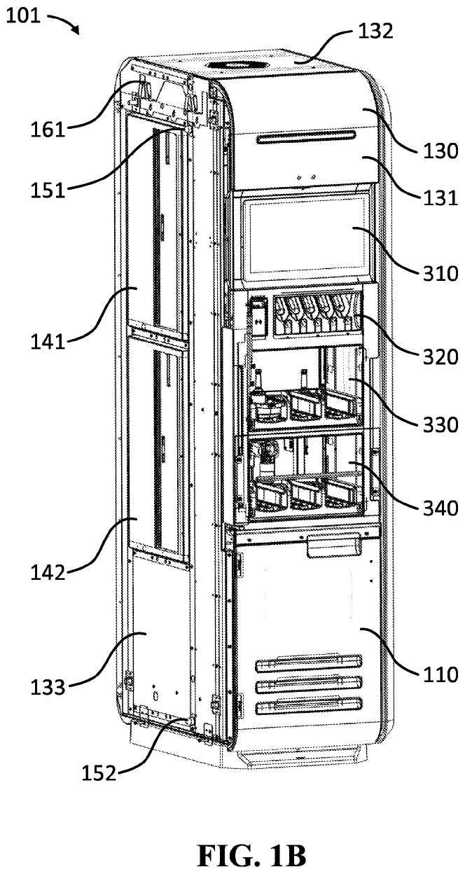

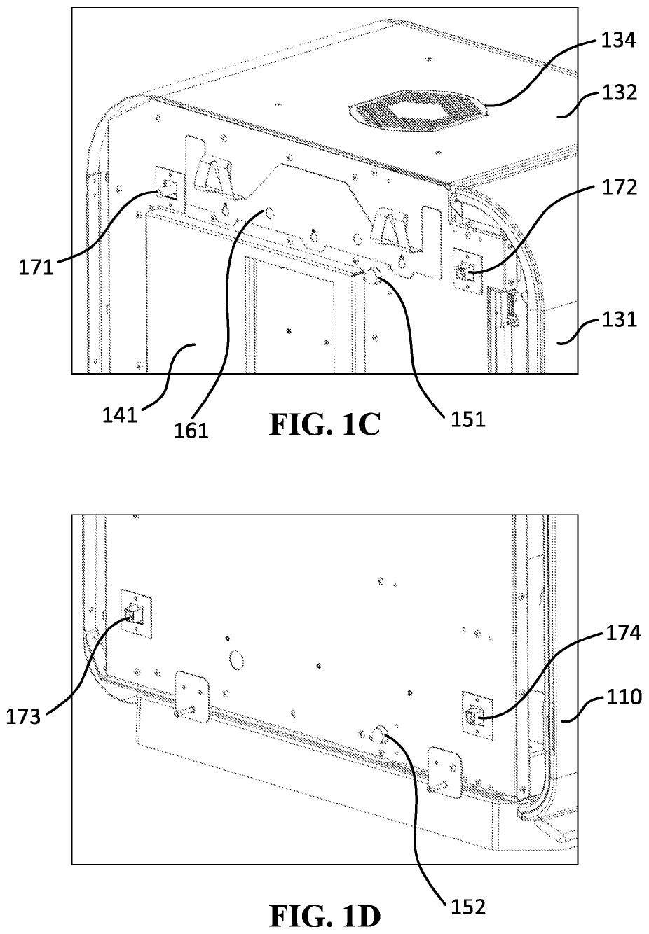

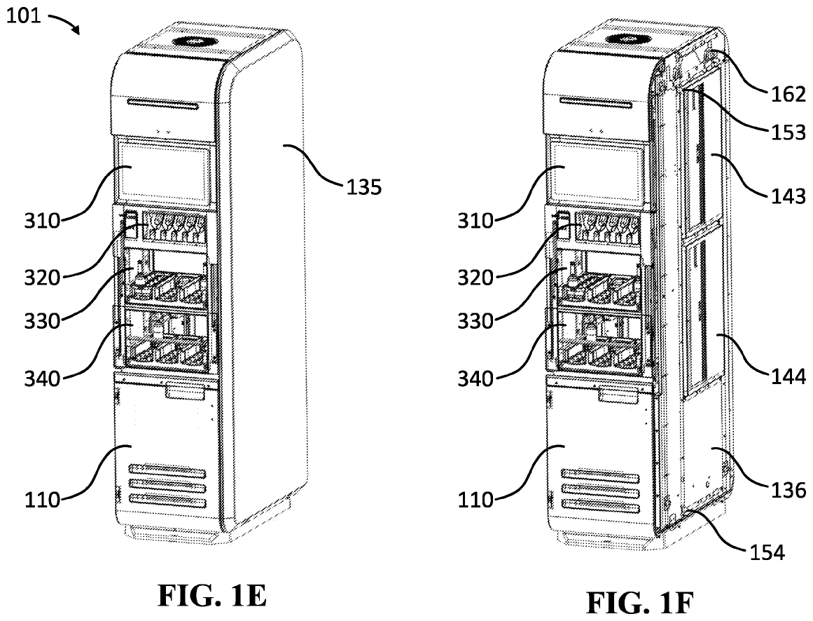

FIG. 1A illustrates an automated system 100 for processing a plurality of sample containers (e.g., blood culture bottles) that includes modules 101 and 102. Module 101 is a sample handling module that is configured to receive sample containers, scan sample containers, transfer sample containers to and from module 102, dispose of sample containers that test negative, and provide sample containers that test positive at an output. As shown, module 101 includes display 310, output chutes 320, compartments 330 and 340, and door 110, which may provide access to a waste receptacle. Module 102 is an incubation and measurement module that is configured to determine whether the sample containers are contaminated with or infected by microorganisms. As shown, module 102 includes doors 121 and 122, which may provide access to drums that hold the sample containers during the incubation and measurement processes.

In some implementations, module 102 may include a high-density drum. High density, as used herein is a description of drum configurations that allow sample containers (e.g., blood culture bottles) to be placed closer to each other to allow a greater number of sample containers to be fitted into the drum compared to the prior art. In some implementations, module 102 may be configured to align the sample containers with a limited number of reader stations. That is, the number of reader stations is less than the number of sample container receptacles in the drum. In some implementations, the drum may be operated by a direct drive motor that can cause accelerated and decelerated drum movement (e.g., a rocking movement, intermittent rotation, etc.). In some implementations, a heater and blower may be provided in the drum housing. The heater and blower circulate warm air around the drum. In some implementations, the heater and blower may be configured to keep the temperature of the contents of all sample containers in the drum within a predetermined narrow range of a specific target temperature. The predetermined narrow range may, for example, be ±1.5° C. of the target temperature. The specific target temperature may be in the range of 30° C. to 40° C. Optionally, the target temperature may be 36.5° C.±0.5° C. Greater temperature uniformity may permit an increase in set point as there is less risk of “over-heating” samples. A greater temperature uniformity at higher temperature will therefore permit a faster time to detection of positive samples. The motor may permit the drum to be positioned such that the user or the automated apparatus can access any sample container carried by the drum. When a sample container is determined to be positive for microbial growth, a workflow may be activated to retrieve that sample container from module 102. Examples of an incubation and measurement module, such as module 102, are described in International Publication No. WO/2021/026272 A1, entitled “High Density Bottle Drum for Storage, Agitation and Reading of Blood Culture Bottles and Methods of Storing,” which is incorporated herein by reference.

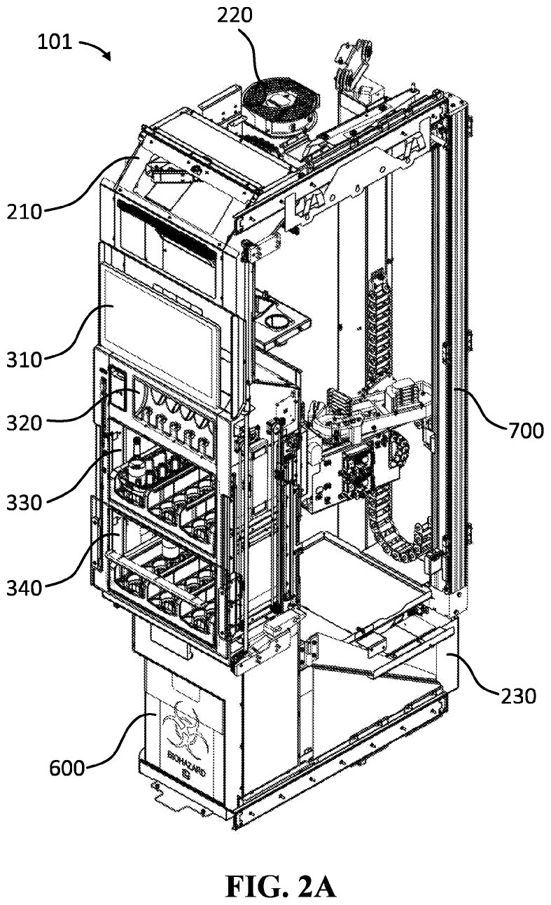

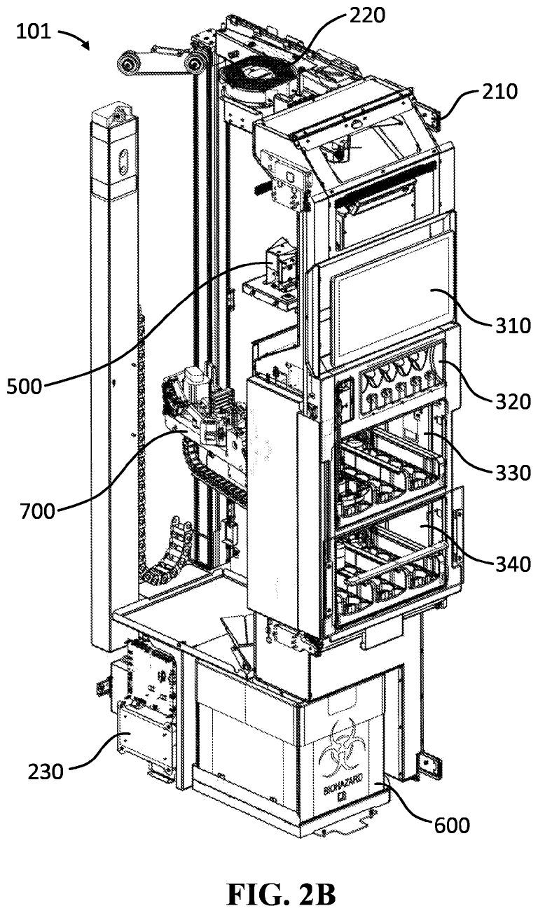

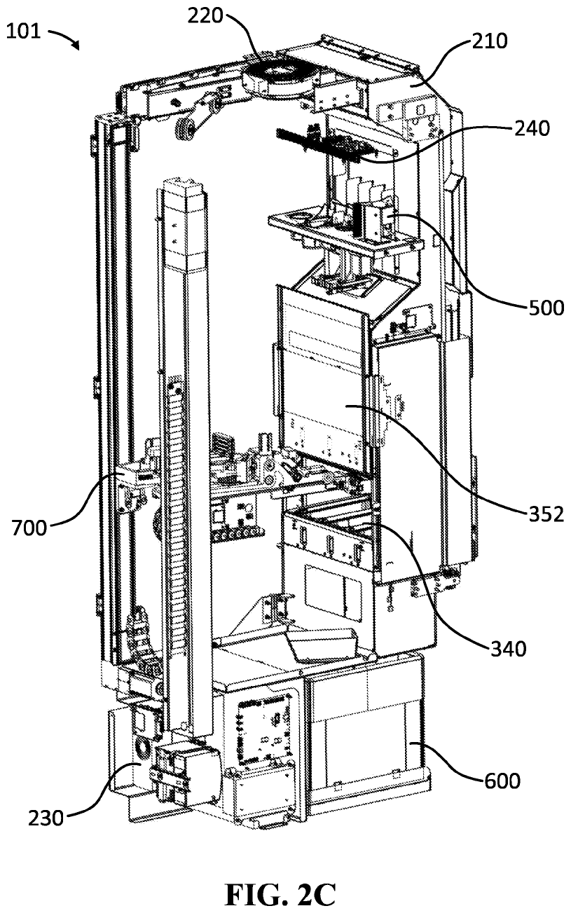

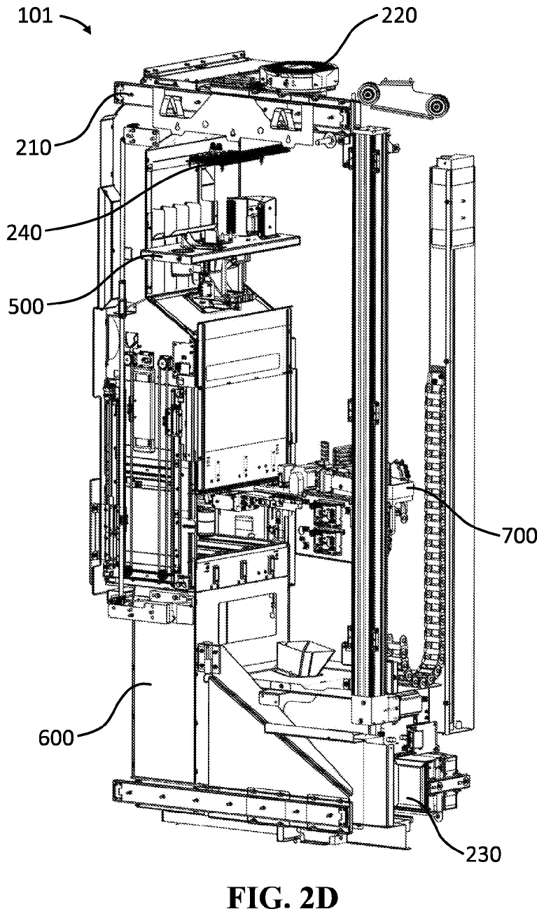

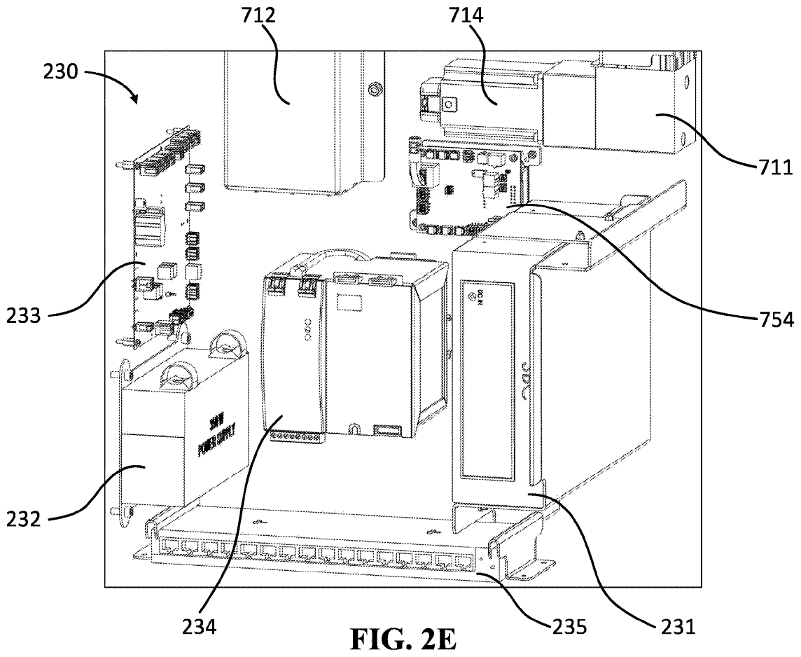

FIG. 1B illustrates module 101 after it has been separated from module 102. As shown, module 101 includes a housing 130, which includes front panel 131, top panel 132, and side panel 133. Side panel 133 includes sliding doors 141 and 142, conical pin guides 151 and 152, and bracket 161. Doors 141 and 142 may be used to access the drums in module 102. For example, door 141 may be used to access a first drum and door 142 may be used to access a second drum. Doors 141 and 142 may also be used to isolate the environmental conditions (e.g., temperature conditions) within module 101 from the environmental conditions within module 102. Guides 151 and 152 may be configured to assist with aligning modules 101 and 102 while they are being assembled together. Bracket 161 may be configured to couple to a cover (not shown). In some implementations, module 101 may also include heating and/or cooling components (see, e.g., fan 220 of FIGS. 2A-2D) to regulate the environmental conditions within module 101.

FIGS. 1C and 1D provide close-up views of top and bottom portions of module 101, respectively. As shown in FIG. 1C, top panel 132 may include a vent 134. As shown in FIGS. 1C and 1D, guides 151 and 152 have a conical shape. A pair of recesses each having a corresponding conical shape may be included in a side panel of module 102. During assembly, guides 151 and 152 may assist a technician with aligning couplings 171-174, which extend through side panel 133, with a corresponding set of couplings extending through a side panel of module 102. For example, during assembly, a technician may position the points of guides 151 and 152 in the corresponding recesses in the side panel of module 102 (not shown). Next, the technician may slide module 101 towards module 102, causing guides to slide along the recesses in the side panel of module 102 and align couplings 171-174 with the corresponding set of couplings extending through the side panel of module 102. In other implementations, guides 151 and 152 may still be pointed, but have a different shape, such as a triangular pyramid shape, a square pyramid shape, or a pentagonal pyramid shape.

As shown in FIG. 1E, module 101 may include a cover 135. As shown in FIG. 1F, cover 135 may be removed to reveal side panel 136, which includes doors 143 and 144, guides 153 and 154, and bracket 162. Side panel 136 and its corresponding components may be structured and/or function much like side panel 133 and its corresponding components. Advantageously, the structure of module 101 allows it to be configured in a few different ways. For example, as shown in FIG. 1A, module 102 may be coupled to the left side of module 101. However, in other implementations, module 102 may be coupled to the right side of module 101. As shown in FIG. 1F, side panel 136 includes doors 143 and 144, which can be used to access the drums in module 102. In some implementations, incubation and measurement modules (e.g., module 102) may be coupled to both sides of module 101. In some implementations, module 101 may be used as a stand-alone unit and covers (e.g., cover 135) may be provided on both sides of module 101.

FIGS. 2A-2D provide perspective views of module 101 without housing 130. As shown, module 101 includes frame 210, fan 220, electronics bay 230, light source 240, display 310, output chutes 320, compartments 330 and 340, imaging subsystem 500, waste management subsystem 600, and robotic subsystem 700. Sample containers (e.g., blood culture bottles) may be received by module 101 in compartments 330 and 340. Robotic subsystem 700 may be configured to transfer sample containers to and/or from module 102, output chutes 320, compartments 330 and 340, imaging subsystem 500, and/or waste management subsystem 600. For example, robotic subsystem 700 may be configured to transfer sample containers to one or more of the drums in module 102 (e.g., through one or more of doors 141-144). As another example, robotic subsystem 700 may be configured to transfer sample containers to and/or from compartments 330 and 340. As yet another example, robotic subsystem 700 may be configured to transfer sample containers to output chutes 320 for retrieval by a user. As yet another example, robotic subsystem 700 may be configured to transfer sample containers to imaging subsystem 500 for scanning. As yet another example, robotic subsystem 700 may be configured to dispose of sample containers in waste management subsystem 600.

Robotic subsystem 700 may also be configured to automatically distribute and/or redistribute sample containers around the circumference of one or more drums in module 102 to distribute the sample containers as desired. For example, robotic subsystem 700 may be configured to move sample containers in module 102 to a specific area of a drum (e.g., an area that can be viewed entirely when door 121 122, or 141-144 is open). This may enable a user to quickly unload those sample containers without having to repeatedly open and close doors 121 and/or 122 and wait for one or more drums in module 102 to rotate. Similarly, this may enable robotic subsystem 700 to quickly retrieve those sample containers without having to repeatedly open and close doors 141-144 and wait for one or more drums in module 102 to rotate. As another example, robotic subsystem 700 may be configured to distribute and/or redistribute sample containers around the circumference of one or more drums in module 102 to balance the drum load (e.g., weight load and/or thermal load). In implementations in which incubation and measurement modules (e.g., module 102) are coupled to both sides of module 101, robotic subsystem 700 may also be configured to automatically distribute and/or redistribute sample containers between the incubation and measurement modules.

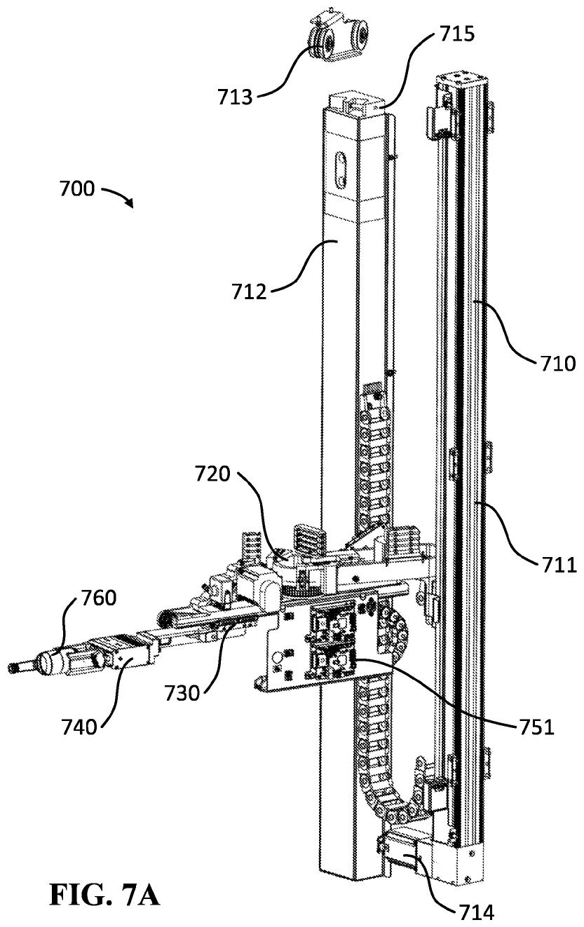

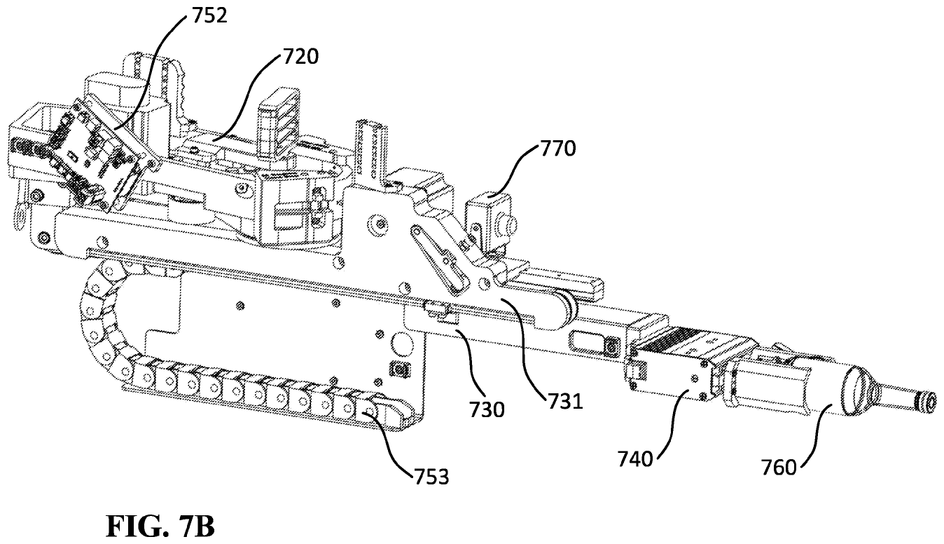

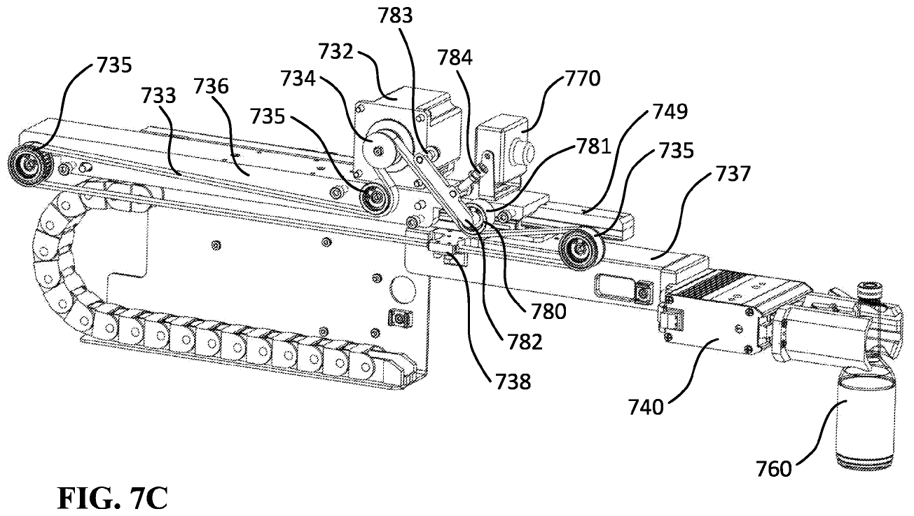

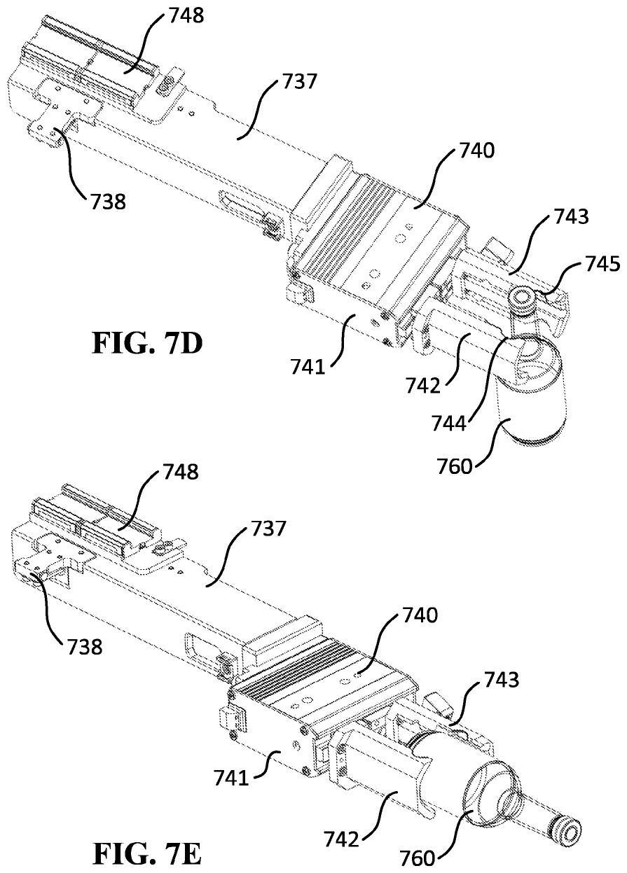

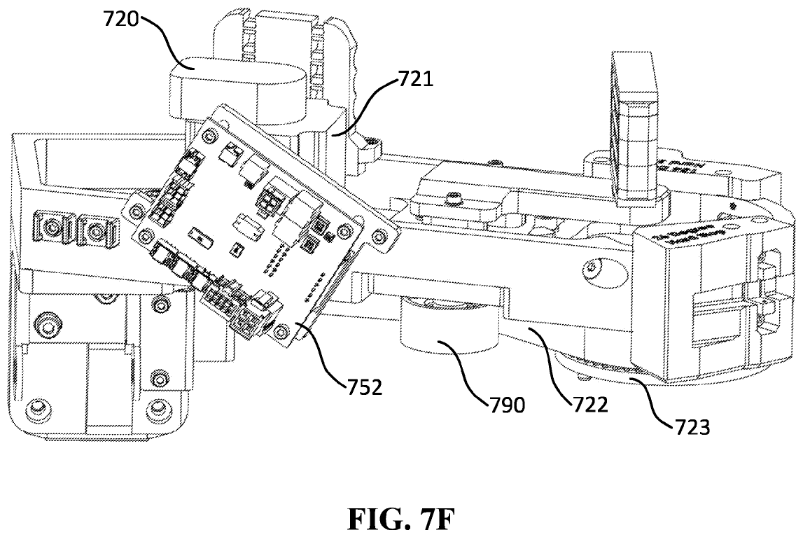

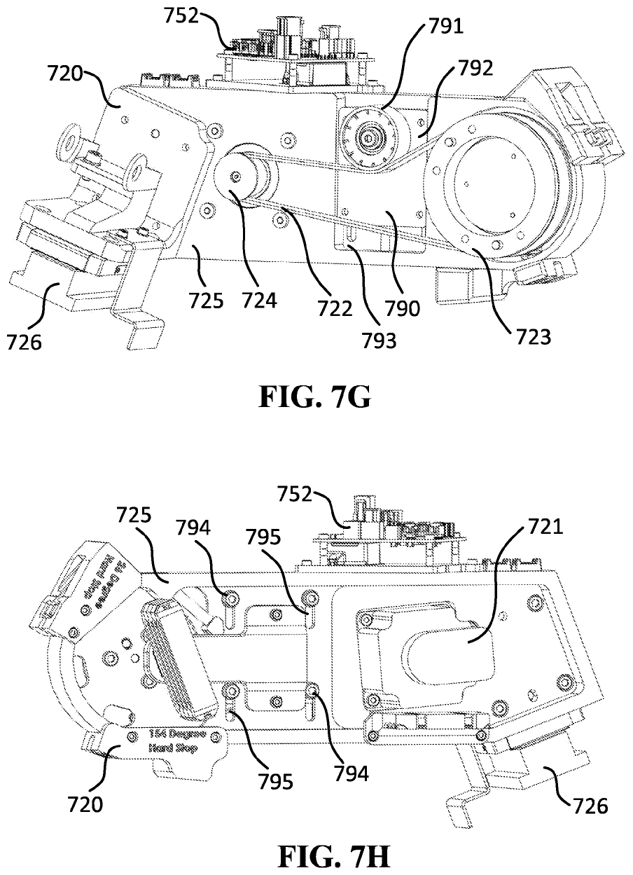

FIG. 2E is a close-up view of electronics bay 230. As shown, computer 231, power supply 232, power distribution board 233, back-up power supply 234, and network switch 235 are positioned in electronics bay 230. Some components of robotic subsystem 700, such as rail 711, counterweight housing 712, motor 714, and controller 754, may also be fully or partially positioned in electronics bay 230. Power distribution board 233, back-up power supply 234, network switch 235, and/or controller 754 may include one or more processors, one or more application specific integrated circuits (ASICs), and/or other similar components. These components may also include a memory medium, such as a hard-drive, memory card, ROM, RAM, DVD, CD-ROM, write-capable, and/or read-only memory, that is capable of storing information. Computer 231 may be communicatively coupled with one or more of the subsystems of module 101. For example, computer 231 may be communicatively coupled to user interface subsystem 300, imaging subsystem 500, waste management subsystem 600, and/or robotic subsystem 700. In some implementations, computer 231 may transmit commands to each of these subsystems and receive measurement data from these subsystems. For example, computer 231 may be configured to control the movements of robotic subsystem 700 and receive measurement data from controllers 751, 752, and 754 and/or camera 770 (see FIGS. 7A-7I). Computer 231 may also be configured to transmit a graphical user interface (GUI), user prompts, user instructions, alerts, system settings, and/or other information to a display.

Power supply 232 may be coupled to an external power source (e.g., an alternating current (AC) wall outlet). Power distribution board 233 may be coupled to power supply 232 and configured to distribute power from power supply 232 to one or more of the subsystems of module 101. Network switch 235 may be communicatively coupled to computer 231 and one or more external devices (e.g., module 102). In some implementations, computer 231 may transmit and receive data using standard communications protocols, such as Inter-Integrated Circuit (I²C), Serial Peripheral Interface (SPI), Controller Area Network (CAN), Universal Asynchronous Reception and Transmission (UART), Ethernet, or Universal Serial Bus (USB), or custom communications protocols. In some implementations, computer 231 may wirelessly transmit and receive data using standard communications protocols, such as Bluetooth, WiFi, ZigBee, Z-Wave, NEC Infrared (IR), Code Division Multiple Access (CDMA), Global System for Mobile Communications (GSM), or Long-Term Evolution (LTE), or custom communications protocols. For example, computer 231 may communicate with one or more internal devices (e.g., controllers 751, 752, 754) using the CAN protocol. As another example, computer may communicate with one or more external devices using the Ethernet protocol. In such implementations, network switch 235 may be an Ethernet switch.

### User Interface Subsystem

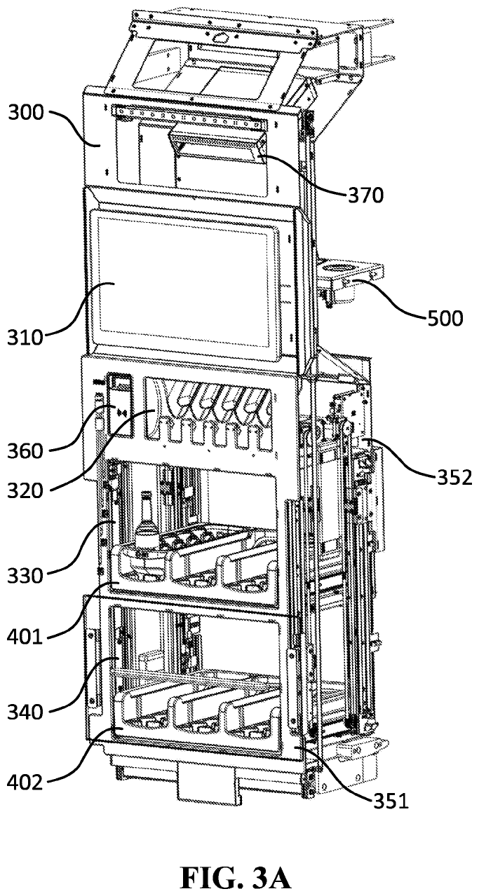

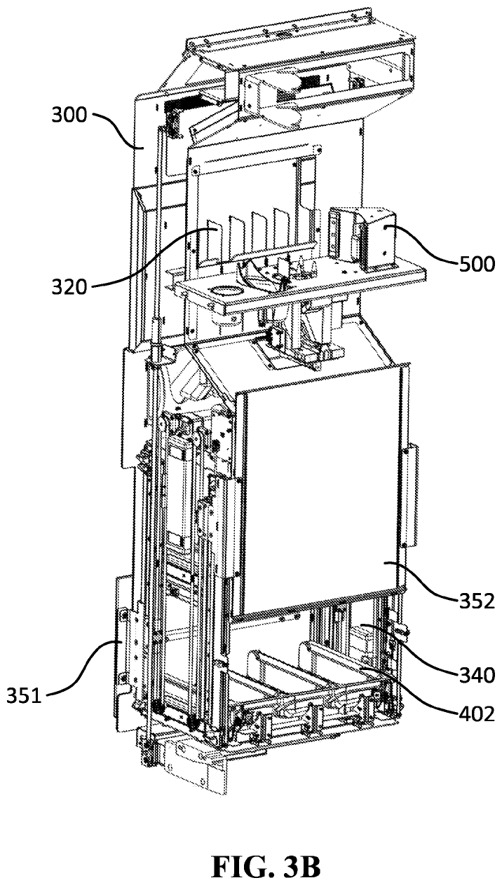

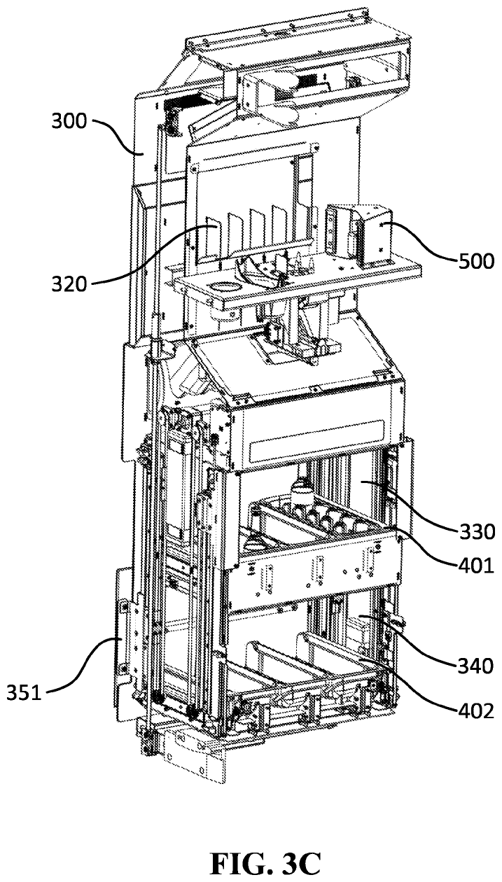

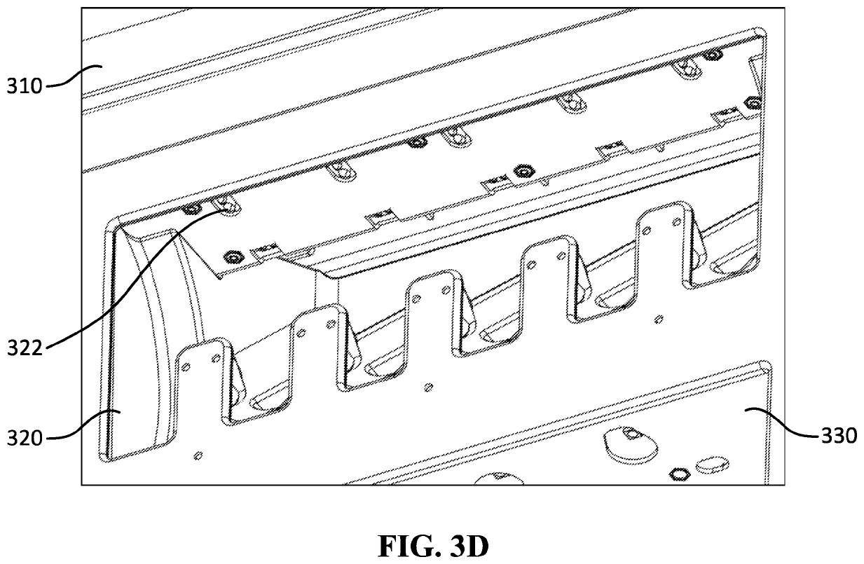

FIG. 3A provides a perspective view of a front side of user interface subsystem 300. As shown, user interface subsystem 300 includes display 310, output chutes 320, indicator lights 322, compartments 330 and 340, doors 351 and 352, reader 360, computer 370, and liners 401 and 402. FIGS. 3B and 3C provide perspective views of a back side of user interface subsystem 300. However, in FIG. 3C, door 352 has been removed to reveal compartment 330 and liner 401. Portions of imaging subsystem 500 are also shown in FIGS. 3A-3C. User interface subsystem 300 and imaging subsystem 500 may be hingedly and/or slidingly connected to frame 210, such that they can be serviced by a technician without moving module 101.

Display 310 may be a touchscreen, monitor, LCD panel, or the like that is configured to display a graphical user interface (GUI), user prompts, user instructions, alerts, system settings, and/or other information that may be relevant to a user. For example, after a batch of sample containers has been received by module 101 and successfully scanned (e.g., by imaging subsystem 500), display 310 may notify the user that the sample containers have been accepted and are being processed. As another example, if the label on a sample container cannot be read, display 310 may display an alert on display 310 that notifies a user of the problem. As yet another example, after a sample container has been deposited into output chutes 320 by robotic subsystem 700, display 310 may be configured to display an arrow above the corresponding chute. Other types of icons, such as a line or a bottle shape, can be used instead of an arrow. In some implementations, the icon may be colored and/or flashing to draw the user's attention. In some implementations, the icon can be pressed to reveal information about the corresponding sample container, such as accession number, sequence number, container type, fill volume, and/or images of the sample container.

In implementations where display 310 is a touchscreen, it may also be configured to receive inputs from the user. In some implementations, inputs from the user may be received from another device that is part of module 101 (e.g., a microphone and/or a keypad) or that is in communication with module 101 (e.g., a mouse and/or keyboard). Regardless of how the user input is received, display 310 may be utilized to implement a load routine and an unload routine. During the load routine, display 310 may ask the user to identify the contents of the sample containers (e.g., controls, empty sample containers, or samples). After the sample containers have been loaded into module 101, they may be digitally tagged with the information provided by the user. During the unload routine, display 310 may request the user to input which sample containers the user would like to unload and/or where the sample containers should be placed (e.g., output chutes 320, compartment 330, or compartment 340).

Output chutes 320 may be used to deliver individual sample containers to a user for retrieval. For example, after a sample is determined to positive in module 102, a user may need to retrieve that sample container for additional microbiological workup. The sample container may be retrieved from module 102 and output from module 101. As shown, there are five output chutes 320. However, in other implementations, module 101 may include more or less output chutes. In some implementations, output chutes 320 may be structured to mitigate the noise made when a bottle is dropped down a chute. In some implementations, output chutes 320 may be constructed with a noise-absorbing material to mitigate the noise made when a bottle is dropped down a chute.

As shown in FIG. 3D, indicator lights 322 are provided above each of output chutes 320. Each one of indicator lights 322 may be configured to illuminate when a sample container is positioned in a particular chute (e.g., the chute below the corresponding light) or when a sample container is positioned in any one of output chutes 320. In some implementations, module 101 may include additional indicator lights by output chutes 320. In some implementations, one or more of indicators lights 322 may be repositioned or removed entirely. For example, one or more of indicator lights 322 may be repositioned below output chutes 320. In some implementations, indicator lights 322 may be different colors, change colors, and/or flash. Furthermore, in some implementations, indicator lights 322 may be configured to behave differently depending on the type of sample container positioned in output chutes 320. For example, indicator lights 322 may illuminate as one color when a sample container is positive (e.g., red) and indicator lights 322 may illuminate as another color when a sample container is negative (e.g., green). In one aspect, the information regarding sample status is transmitted from a sample status (i.e., positive or negative) indicator in module 102. The status of a sample container may be associated with a barcode on the sample container and that barcode information may be read when the sample container is retrieved from module 102 to determine the output chute, tray, or receptable in which to place the retrieved sample container.

In some implementations, output chutes 320 may include one or more sensors that are configured to detect when a sample container has been deposited into output chutes 320. This detection may be used to trigger the illumination of indicator lights 322. It may also be used to provide feedback to robotic subsystem 700. Such feedback may advantageously prevent robotic subsystem 700 from depositing another sample container down the same chute and causing a crash and/or contamination. In some implementations, the one or more sensors may be positioned at the bottoms of each of output chutes 320. In some implementations, the one or more sensors may be touch sensors, optical sensors, and/or ultrasonic sensors.

Compartments 330 and 340 may be used for both input and output purposes. For example, while a user is loading compartment 330 with untested sample containers, robotic subsystem 700 may be depositing sample containers in compartment 340 for retrieval by a user. Similarly, while a user is loading compartment 340 with untested sample containers, robotic subsystem 700 may be depositing sample containers in compartment 330 for retrieval by a user. Alternatively, compartments 330 and 340 may be simultaneously utilized as an input area or an output area. For example, a user may load untested sample containers into both of compartments 330 and 340. As another example, robotic subsystem 700 may deposit sample containers into both of compartments 330 and 340 for retrieval by a user. As a result, in the particular implementation of FIGS. 3A-3C, a user can load or unload up to 60 sample containers at the same time. In other implementations, module 101 may include more or less compartments. For example, a third compartment structured like compartments 330 and 340 may be added to module 101 to increase the number of sample containers that can be loaded and unloaded at a single time to 90.

In some implementations, compartments 330 and 340 may be utilized as the primary output areas and output chutes 320 may be utilized as the secondary output area. For example, in such implementations, batches of sample containers that tested positive may be deposited into compartments 330 and/or 340 by robotic subsystem 700. Having a designated area where batches of positives are presented (separate from negatives) saves time and reduces the risk of user error (e.g., due to inaccurate sorting). Furthermore, in such implementations, output chutes 320 may be utilized for outputting individual sample containers, sample containers requiring additional information and/or sample containers requiring system error resolution. For example, when imaging subsystem 500 is unable to read a barcode label on a sample container, robotic subsystem 700 may deposit that sample container into one of output chutes 320. This allows a user to resolve the problem by, for example, removing obstructions from the label or applying a new bar code label. In other implementations, compartments 330 and/or 340 may be utilized as the secondary output areas and output chutes 320 may be utilized as the primary output area.

In some implementations, a user can designate whether output chutes 320, compartment 330, and compartment 340 are utilized as the primary and/or secondary output areas. For example, display 310 can be utilized to access system settings that allow a user to designate where untested sample containers are received by module 101 and where sample containers are deposited by robotic subsystem 700 for retrieval by a user. Through these system settings, a user may also be able to specify whether the sample containers are output into a removeable rack. In such implementations, if no rack is detected, display 310 may alert the user.

The input and output areas described above (e.g., output chutes 320 and compartments 330 and 340) are particularly advantageous due to their flexibility. For example, since compartments 330 and 340 can be utilized as input or output areas, a large number of sample containers can be loaded or unloaded at a single time. Furthermore, some laboratories may prefer to use compartments 330 and 340 as the primary output area and others may prefer to use output chutes 320 as the primary output area. For example, sample containers may make noise as they descend output chutes 320, which may convey the impression of poor quality. Placing sample containers in compartments 330 and/or 340 for retrieval avoids such noises. Additionally, when placed in racks, large numbers of sample containers can be quickly unloaded by a user from compartments 330 and/or 340.

As shown in FIG. 3A, compartment 330 includes liner 401 and compartment 340 includes liner 402. In some implementations, one or both of liners 401 and 402 may be a piece of molded plastic that has cylindrically shaped receptacles designed to accept sample containers (e.g., blood culture bottles) directly. In some implementations, one or more receptables may be structured to prevent a sample container from falling over after being placed in a receptable. One or both of liners 401 and 402 may also include recesses designed to accept racks of sample containers. In some implementations, liners 401 and/or 402 may include acrylonitrile-butadiene-styrene (ABS), polypropylene (PP), polystyrene (PS), another type of plastic, and/or a mix thereof.

Liners 401 and 402 may advantageously enable a user to load a variable number of sample containers into module 101. Liners 401 and 402 may also advantageously enable a user to load individual sample containers or racks of sample containers into module 101. Having a flexible input area such as this allows users to maintain the same workflow for loading small (e.g., 1-2 sample containers), medium, and large (e.g., 20+ sample containers) batches of sample containers. There are separate advantages to loading sample containers individually and loading sample containers in racks. For example, racks provide a visual cue for users to transfer sample containers to and/or from module 101. This may allow a laboratory to develop Standard Operating Procedures (SOP) around batch loading. Furthermore, it is quicker to load a plurality of sample containers into module 101 by simply placing a rack in module 101, as opposed to individually placing each sample container in module 101. Moreover, studies have shown that racks add a layer of safety when transporting sample containers within a laboratory. However, studies have also shown that users often forget to reload racks into laboratory instruments. Additionally, not all laboratories prefer to use racks. Many do not prefer the usage of racks for reasons including extra time spent managing racks, labelling them, handling the loss, damage and reordering of racks, and cleaning of the racks. Therefore, having a system that accepts individual sample containers or racks of sample containers is particularly advantageous.

In some implementations, liner 401 may be removably coupled to a first drawer (not shown) and/or liner 402 may be removably coupled to a second drawer (not shown). The one or more drawers may include a flat shelf that partially or fully slides out of module 101 using drawer ball-bearing slide rails or another similar mechanism. This is advantageous as it allows the user to see the cylindrical recesses and allows the sample containers to be placed from above, which enhances the usability and ergonomics of loading and unloading sample containers. In some implementations, one or both of liners 401 and 402 may be removed from a drawer by a user by use of a thumb screw, a latch, or other similar fastener. After a liner has been removed, it can be easily cleaned (e.g., in a bleach solution soaking tub). Furthermore, while a liner is being cleaned, another liner can be placed in module 101 to limit instrument down-time. In some implementations, one or more receptacles in liners 401 and/or 402 may include a hole at the bottom of the receptacle. When liners 401 and/or 402 are removed from module 101, these holes may allow cleaning fluids to drain through. Furthermore, when liners 401 and/or 402 are positioned in module 101, these holes may allow one or more sensors to detect the presence or absence of sample containers. In some implementations, the one or more sensors may be touch sensors, optical sensors, and/or ultrasonic sensors.

In some implementations, compartments 330 and/or 340 may include indicator lights (not shown). The indicator lights may be positioned above liners 401 and/or 402. These indicator lights may be used to signal to a user how compartments 330 and/or 340 are being utilized. For example, when compartment 330 is being utilized as an input area, one or more indicator lights may illuminate compartment 330 with a first color (e.g., blue or green), and when compartment 330 is being utilized as an output area, the one or more indicator lights may illuminate compartment 330 with a second color (e.g., red). Similarly, when compartment 340 is being utilized as an input area, one or more indicator lights may illuminate compartment 340 with a first color (e.g., blue or green), and when compartment 340 is being utilized as an output area, the one or more indicator lights may illuminate compartment 340 with a second color (e.g., red). The one or more indicator lights in compartments 330 and/or 340 may also be used indicate whether a batch of sample containers is ready for retrieval. For example, while robotic subsystem 700 is depositing sample containers into one of compartments 330 and 340, the compartment may be illuminated with a first color (e.g., red), and when the batch of sample containers is ready for retrieval, the compartment may be illuminated with a second color (e.g., blue or green). In some implementations the indicator lights may flash instead of changing colors.

As shown in FIGS. 3A-3C, door 351 may be positioned on the front side of user interface subsystem 300, and door 352 may be positioned on the back side of user interface subsystem 300. Doors 351 and 352 are vertical sliding doors configured to be raised and lowered to expose either one of compartments 330 and 340. For example, when doors 351 and 352 are in a raised position, compartment 330 is sealed and compartment 340 is exposed. Similarly, when doors 351 and 352 are in a lowered position, compartment 340 is sealed and compartment 330 is exposed. In some implementations, doors 351 and 352 may be configured to maintain positions opposite one another. For example, when door 351 is in a raised position, door 352 is in a lowered position. Similarly, when door 351 is in a lowered position, door 352 is in a raised position. This may prevent a user from mixing untested sample containers with sample containers that have tested positive. For example, while robotic subsystem 700 is depositing positive sample containers into compartment 330, door 351 may be in a raised position to prevent a user from placing untested sample containers in compartment 330. Furthermore, door 352 may help prevent sample containers from falling into module 101 (e.g., onto support structure 641 of waste management subsystem 600) while a user loads untested sample containers into one of compartments 330 and 340.

In some implementations, one or both of doors 351 and 352 may be constructed with a transparent material, such as plastic or glass. A transparent material advantageously permits a user to observe the processing of the sample containers, which may provide increased confidence in the system. For example, a transparent door allows users to see the sample containers being loaded and unloaded, which increases their overall understanding of how the system works, their visibility to potential jams/errors, and potentially their overall trust in the instrument. As shown, door 351 is constructed with a transparent material and door 352 is constructed with an opaque material. However, in other implementations, both of doors 351 and 352 may be constructed with a transparent material. Furthermore, in other implementations, both of doors 351 and 352 may be constructed with an opaque material.

In other implementations, doors 351 and 352 may be replaced with one or more swinging doors. For example, door 351 may be replaced with a pair of swinging doors, one of which is configured to seal compartment 330 and one of which is configured to seal compartment 340. In such implementations, one or both of the swinging doors may be locked to prevent a user from accessing the corresponding compartment. For example, while robotic subsystem 700 is depositing positive sample containers into compartment 330, the swinging door positioned in front of compartment 330 may be locked. Similarly, while robotic subsystem 700 is depositing positive sample containers into compartment 340, the swinging door positioned in front of compartment 340 may be locked.

As shown in FIG. 3A, reader 360 may be configured to read barcode labels, RFID tags, and/or other types of identifiers on sample containers and/or user identification cards. For example, a user can scan a sample container using reader 360 to lookup information for that sample container (e.g., a sequence number or accession number). In some implementations, the information may be provided on display 310. In some implementations, after scanning a sample container with reader 360, a user can manually load the sample container into an incubation and measurement module, such as module 102. In some implementations, a user can place his or her employee badge or other unique identification card in proximity to the reader 360 to initiate an automatic login. To protect patient information and comply with cyber security regulations, a user may need to login to perform certain actions, such viewing test results and obtaining positive sample containers. In some implementations, a user can place his or her employee badge or other unique identification card in proximity to the reader 360 to automatically adjust one or more system settings. For example, module 101 may store user preferences regarding where sample containers are deposited for retrieval and automatically adjust the system settings to those preferences after the user scans his or her unique identification card.

In some implementations, separate readers may be provided for reading identifiers on sample containers and for reading user identification cards. In some implementations, the one or more readers may use different scanning technologies. For example, an optical bar-code reader could be used to scan the labels on sample containers and a radio frequency identification (RFID) reader could be used to scan user identification cards. In some implementations, a user may also login via other methods, such as a username and password, a passphrase, a PIN, and/or a picture password. In some implementations, a user may also login via a biometric alternative, such as voice, facial, retinal, and/or fingerprint recognition.

As shown in FIG. 3A, computer 370 may include one or more processors, one or more application specific integrated circuits (ASICs), and/or other similar components. Computer 370 may also include a memory medium, such as a hard-drive, memory card, ROM, RAM, DVD, CD-ROM, write-capable, and/or read-only memory, that is capable of storing information. Computer 370 may be communicatively coupled with one or more of the subsystems of module 101. For example, computer 370 may be communicatively coupled to one or more components of electronics bay 230 (e.g., computer 231), user interface subsystem 300, imaging subsystem 500, waste management subsystem 600, and/or robotic subsystem 700. In some implementations, computer 370 may receive commands from one or more of these subsystems. In some implementations, computer 370 may transmit commands to one or more of these subsystems and receive measurement data from one or more of these subsystems. For example, computer 370 may be configured to control the movements of robotic subsystem 700 and receive measurement data from controllers 751 and 752 and/or camera 770 (see FIGS. 7A-71). Computer 370 may also be configured to transmit a graphical user interface (GUI), user prompts, user instructions, alerts, system settings, and/or other information to display 310 for display.

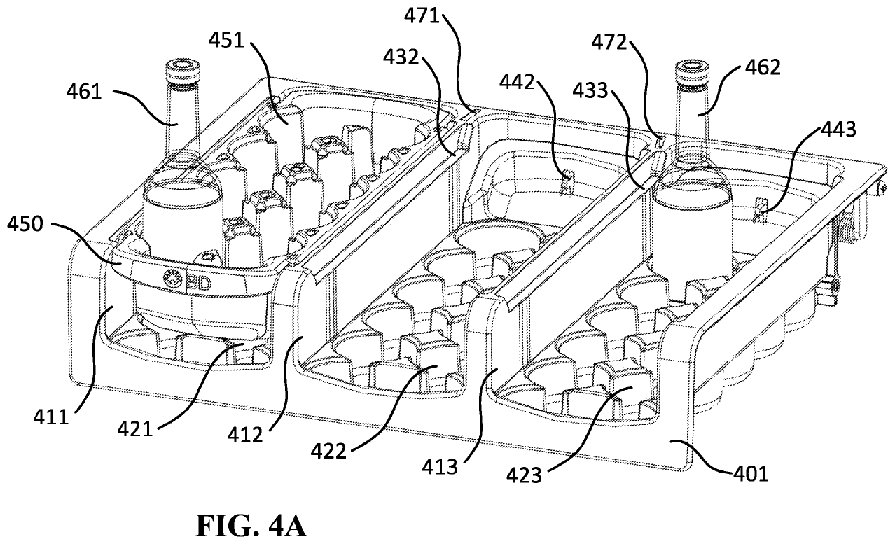

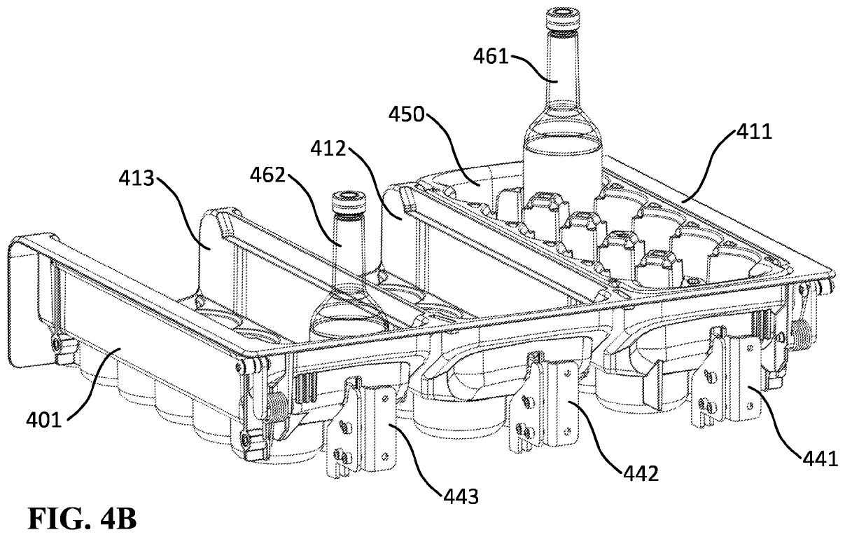

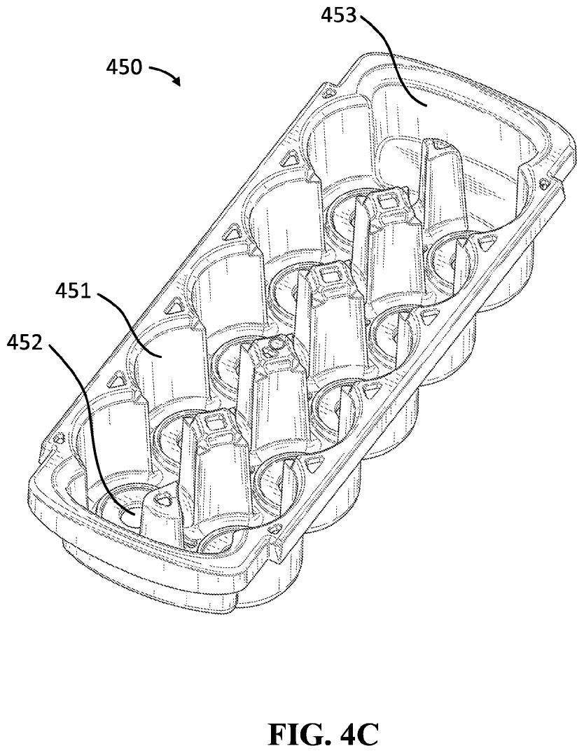

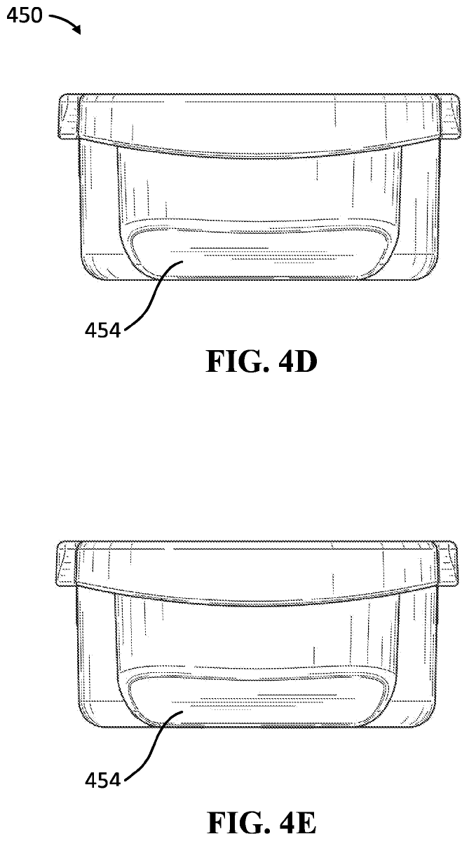

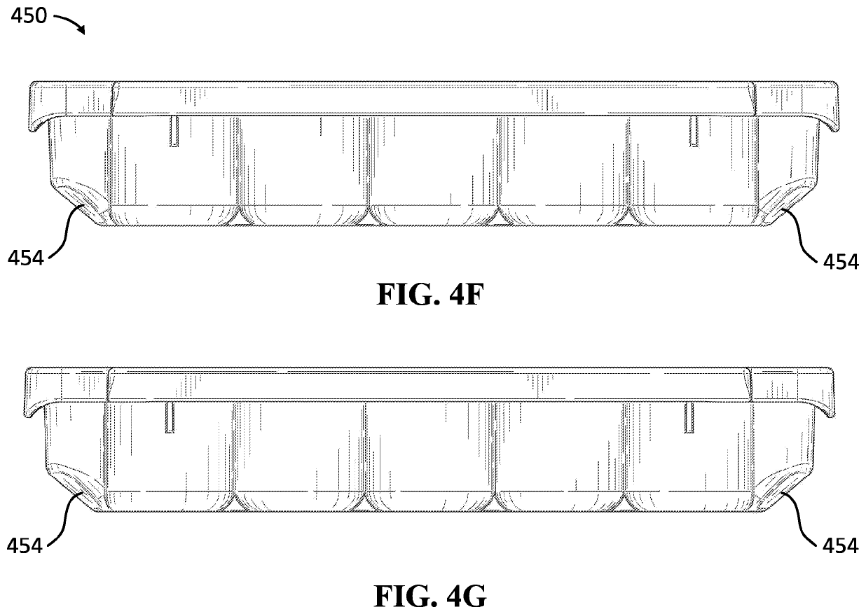

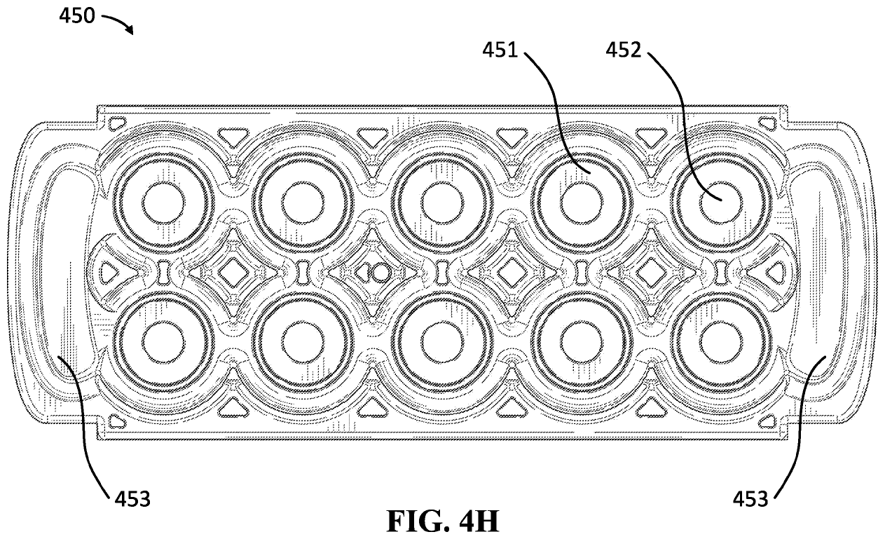

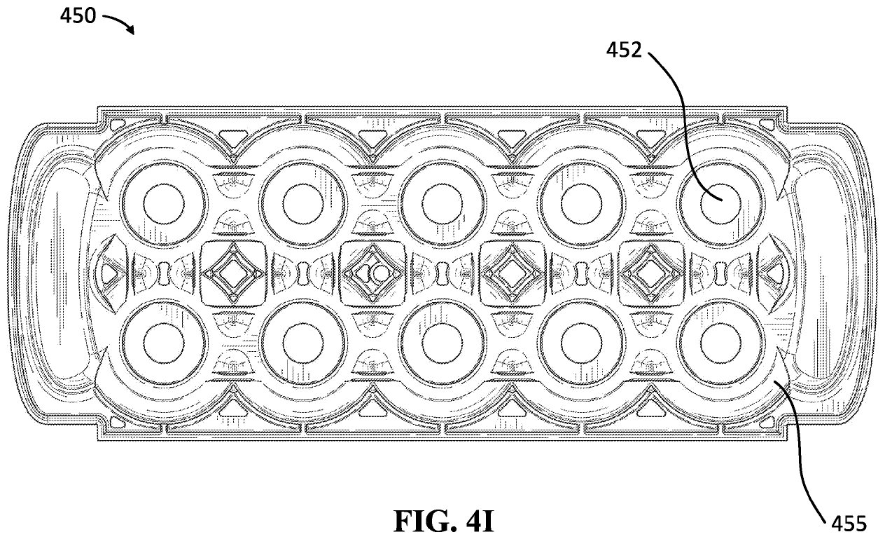

FIGS. 4A and 4B provide perspective views of liner 401. As shown, liner 401 includes sections 411-413. Sensors 441-443 extend through sections 411-413, respectively, and may be configured to detect whether a rack is present. In some implementations, sensors 441-443 may be touch sensors, optical sensors, and/or ultrasonic sensors. Section 411 includes receptacles 421 designed to accept sample containers (e.g., blood culture bottles) directly and recesses (not shown) designed to accept racks of sample containers, such as rack 450. FIGS. 4C-4I illustrate different views of rack 450. More specifically, FIG. 4C is a perspective view of rack 450, FIG. 4D is a front elevation view thereof, FIG. 4E is a back elevation view thereof, FIG. 4F is a right-side elevation view thereof, FIG. 4G is a left-side elevation view thereof, FIG. 4H is a top-down view thereof, and FIG. 4I is a bottom-up view thereof.

As shown in FIGS. 4A and 4B, a single sample container 461 is positioned in one of the receptacles 451 of rack 450. Section 412 includes receptacles 422 designed to accept sample containers directly and recesses 432 designed to accept racks of sample containers. Section 413 includes receptacles 423 designed to accept sample containers directly, such as sample container 462, and recesses 433 designed to accept racks of sample containers. As shown, receptacles 421-423 and/or 451 may include a cylindrical chamfer feature to facilitate the insertion of a sample container into the corresponding receptacle. One or more of receptacles 421-423 and/or 451 may also include a hole (e.g., holes 452 in FIGS. 4C, 4H, and 4I) at the bottom of the receptacle. During cleaning, these holes may allow cleaning fluids to drain through. As shown, receptacles 421-423 and/or 451 may have bottoms with rounded edges to accommodate rocker bottles. A rocker bottle has a convex bottom due to pressure build up or heat in manufacturing. Inspection and controls are in place to prevent this, but some bottles make it through with up to, for example, a 1.5 mm convex shape.

As shown in FIGS. 4A and 4B, each one of sections 411-413 can hold up to ten sample containers in the corresponding receptacles. Rack 450 can also hold up to ten sample containers in receptacles 451. However, in other implementations, sections 411-413 and/or rack 450 may be configured to hold more or less sample containers. Similarly, in other implementations, liner 401 may include more or less sections of receptacles. For example, liner 401 may only include two sections, each of which can hold up to fifteen sample containers. In such implementations, rack 450 may also be reconfigured to hold up to fifteen sample containers.

As shown in FIGS. 4A and 4B, receptacles 421-423 and 451 are configured to accept bottles having a particular diameter. For example, receptacles 421-423 and 451 may be configured to accept specific types of culture bottles, such as the BD BACTEC™ culture bottles, which are manufactured and sold by Becton, Dickinson and Company. However, in other implementations, receptacles 421-423 and 451 may be shaped differently and configured to hold different types of sample containers. Furthermore, in some implementations, receptacles 421-423 and 451 may be configured to accept a range of sample containers with differing diameters and/or heights. For example, one or more of receptacles 421-423 and 451 may have lower and upper portions with different diameters. The lower portion may, for example, have a narrow diameter for accepting sample containers with a similarly narrow diameter. Furthermore, the upper portion may, for example, have a wide diameter for accepting sample containers with a similarly wide diameter.

In some implementations, liner 401 may include one or more features for calibrating robotic subsystem 700. For example, liner 401 may include notches 471 and 472. In some implementations, a pin (not shown) may be temporarily or permanently added to robotic subsystem 700. For example, during a calibration procedure, a pin may be screwed into a component of gripper assembly 740. In some implementations, the pin may remain attached to robotic subsystem 700 during the processing of sample containers. The pin may be sized to fit within notches 471 and 472. After engaging one of notches 471 and 472 with the pin, the positions of one or more components of robotic subsystem 700 may be saved in memory and used as a point of reference for future movements of robotic subsystem 700.

As best seen in FIGS. 4C-4I, rack 450 can be advantageously picked up with one hand by a user. For example, a user may position his or her thumb in one of recesses 453 while simultaneously resting the tips of his or her fingers along one of slanted surfaces 454. The user may then slide his or her fingers along the slanted surface 454 until they contact bottom surface 455 of rack 450. Once in this position, the user can securely lift rack 450. Advantageously, the symmetrical shape of recesses 453 and slanted surfaces 454 allows a user to lift rack 450 with his or her right hand or left hand.

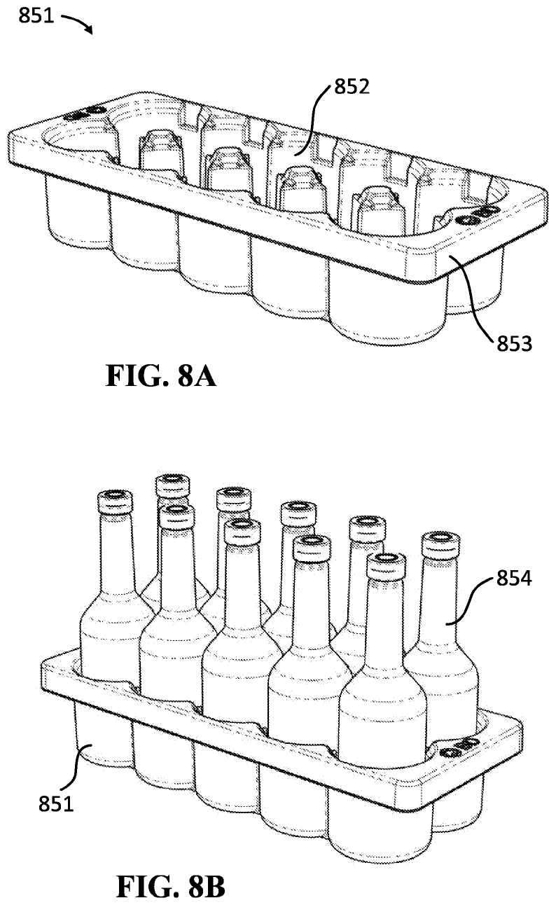

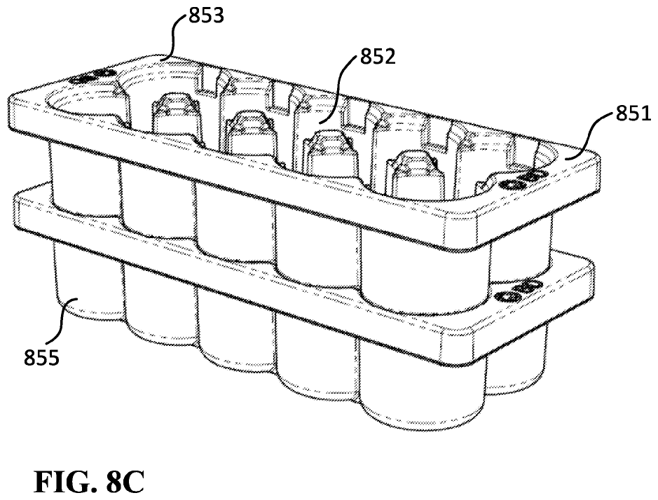

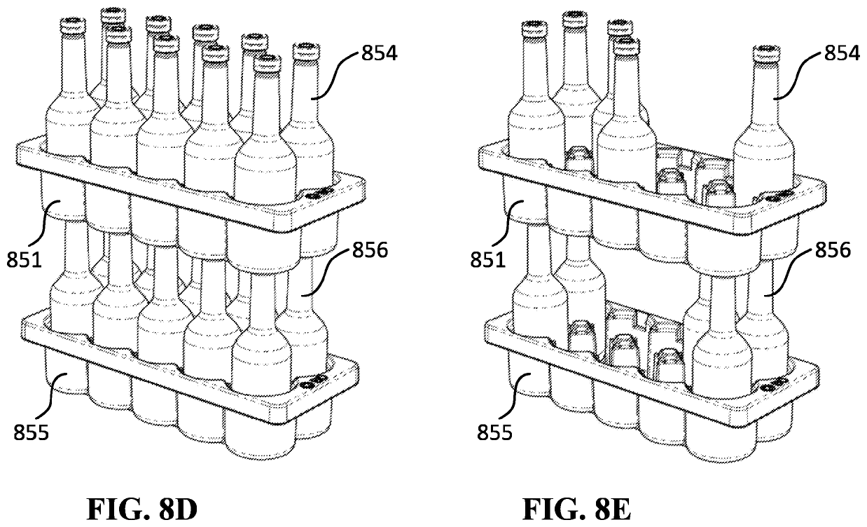

FIGS. 8A-8E illustrate another implementation of a rack that may be compared to rack 450. As shown in FIG. 8A, rack 851 includes receptacles 852 and lip 853. Receptacles 852 may include a cylindrical chamfer feature to facilitate the insertion of a sample container into the corresponding receptacle. One or more of receptacles 852 may include a hole at the bottom of the receptacle. During cleaning, these holes may allow cleaning fluids to drain through. When positioned in a liner in module 101, these holes may allow one or more sensors to detect the presence or absence of sample containers. In some implementations, the one or more sensors may be touch sensors, optical sensors, and/or ultrasonic sensors. Lip 853 acts as a handle for a user to grasp. As shown in FIG. 8B, receptacles 852 are designed to accept sample containers 854.

As shown in FIG. 8C, rack 851 is stackable on top of another rack 855 using complementary locating features (e.g., countersinks and center post structures). For example, the bottom of rack 851 may include a set of locating features and the top of rack 855 may include a complementary set of locating features to enable rack 851 to be securely stacked on top of rack 855. As shown in FIGS. 8D and 8E, rack 851 can also be stacked on top of rack 855 while sample containers 856 are positioned in rack 855. In such implementations, the bottom of rack 851 may include counterbore features that mate into a complementary feature of sample containers 856. For example, if sample containers 856 are blood culture bottles, rack 851 may include counterbore features that mate into the crimp ring and septum of a blood culture bottle.

As shown in FIGS. 8A-8E, racks 851 and 855 can hold up to ten sample containers. However, in other implementations, racks 851 and 855 may be configured to hold more or less sample containers. Furthermore, as shown in FIGS. 8A-8E, receptacles 852 are configured to accept bottles having a particular diameter. For example, receptacles 852 may be configured to accept specific types of culture bottles, such as the BD BACTEC™ culture bottles, which are manufactured and sold by Becton, Dickinson and Company. However, in other implementations, receptacles 852 may be shaped differently and configured to hold different types of sample containers. Furthermore, in some implementations, receptacles 852 may be configured to accept a range of sample containers with differing diameters. For example, one or more of receptacles 852 may have lower and upper portions with different diameters. The lower portion may, for example, have a narrow diameter for accepting sample containers with a similarly narrow diameter. Furthermore, the upper portion may, for example, have a wide diameter for accepting sample containers with a similarly wide diameter.

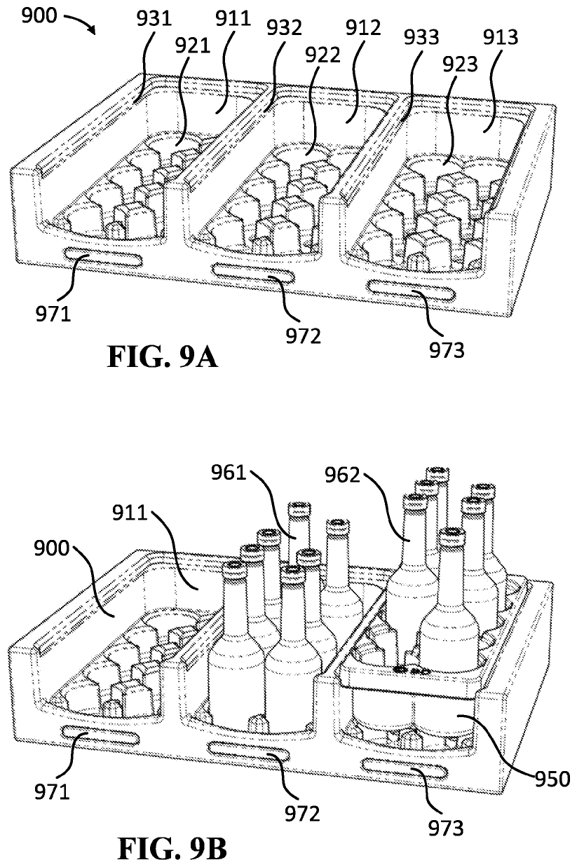

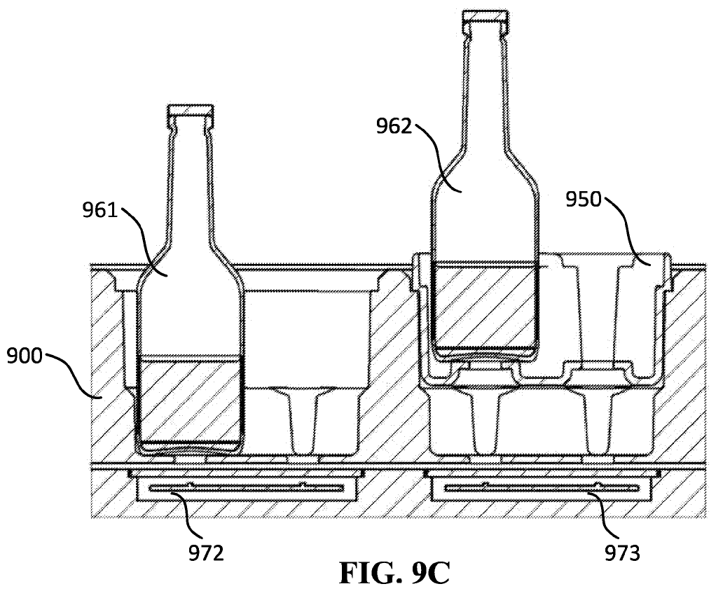

FIGS. 9A-9C illustrate another implementation of a liner that may be compared to liners 401 and 402. As shown, liner 900 includes sections 911-913. Section 911 includes receptacles 921 designed to accept sample containers (e.g., blood culture bottles) directly and recesses 931 designed to accept racks of sample containers. Section 912 includes receptacles 922 designed to accept sample containers directly, such as sample containers 961, and recesses 932 designed to accept racks of sample containers. Section 913 includes receptacles 923 designed to accept sample containers directly and recesses 933 designed to accept racks of sample containers, such as rack 950 with sample containers 962. As shown, receptacles 921-923 may include a cylindrical chamfer feature to facilitate the insertion of a sample container into the corresponding receptacle. One or more of receptacles 921-923 may include a hole at the bottom of the receptacle. During cleaning, these holes may allow cleaning fluids to drain through. When positioned in module 101, these holes may allow one or more sensors to detect the presence or absence of sample containers. In some implementations, the one or more sensors may be touch sensors, optical sensors, and/or ultrasonic sensors.

Indicator lights 971-973 are positioned in front of sections 911-913, respectively. In some implementations, indicator lights 322 may be different colors, change colors, and/or flash. In some implementations, one or more of indicator lights 971-973 may be configured to illuminate based on the status of the corresponding section. For example, when section 911 is empty and ready to receive a batch of sample containers, indicator light 971 may change to a first predetermined color. As another example, when section 911 is full with untested sample containers, indicator light 971 may change to a second predetermined color. As yet another example, when section 911 contains positive sample containers, indicator light 971 may change to a third predetermined color. Indicator lights 972 and 973 may also behave in a similar manner based on the status of sections 912 and 913, respectively. In some implementations, liner 900 may include additional indicator lights. In some implementations, one or more of indicators lights 971-973 may be repositioned or removed entirely. For example, one or more of indicator lights 971-973 may be repositioned in the bottoms of receptacles 921-923.

As shown in FIGS. 9A-9C, each one of sections 911-913 can hold up to ten sample containers in the corresponding receptacles. However, in other implementations, sections 911-913 may be configured to hold more or less sample containers. Similarly, in other implementations, liner 900 may include more or less sections of receptacles. For example, liner 900 may only include two sections, each of which can hold up to fifteen sample containers. In such implementations, rack 950 may also be reconfigured to hold up to fifteen sample containers.

As shown in FIGS. 9A-9C, receptacles 921-923 are configured to accept bottles having a particular diameter. For example, receptacles 921-923 may be configured to accept specific types of culture bottles, such as the BD BACTEC™ culture bottles, which are manufactured and sold by Becton, Dickinson and Company. However, in other implementations, receptacles 921-923 may be shaped differently and configured to hold different types of sample containers. Furthermore, in some implementations, receptacles 921-923 may be configured to accept a range of sample containers with differing diameters. For example, one or more of receptacles 921-923 may have lower and upper portions with different diameters. The lower portion may, for example, have a narrow diameter for accepting sample containers with a similarly narrow diameter. Furthermore, the upper portion may, for example, have a wide diameter for accepting sample containers with a similarly wide diameter.

### Imaging Subsystem

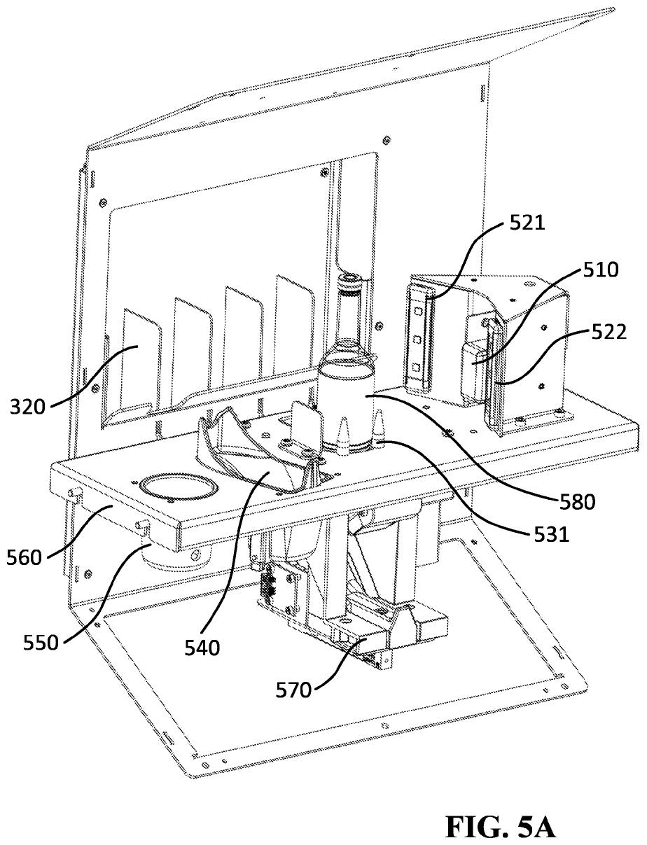

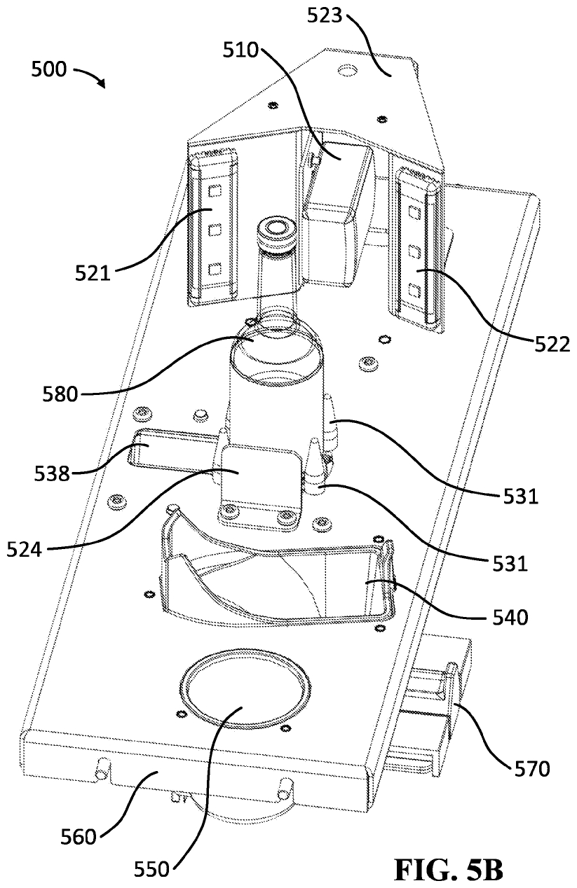

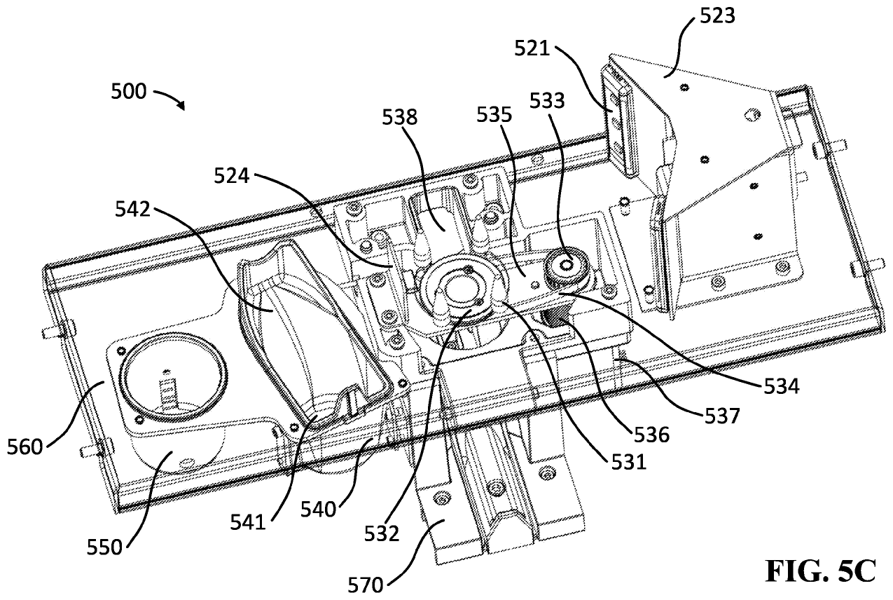

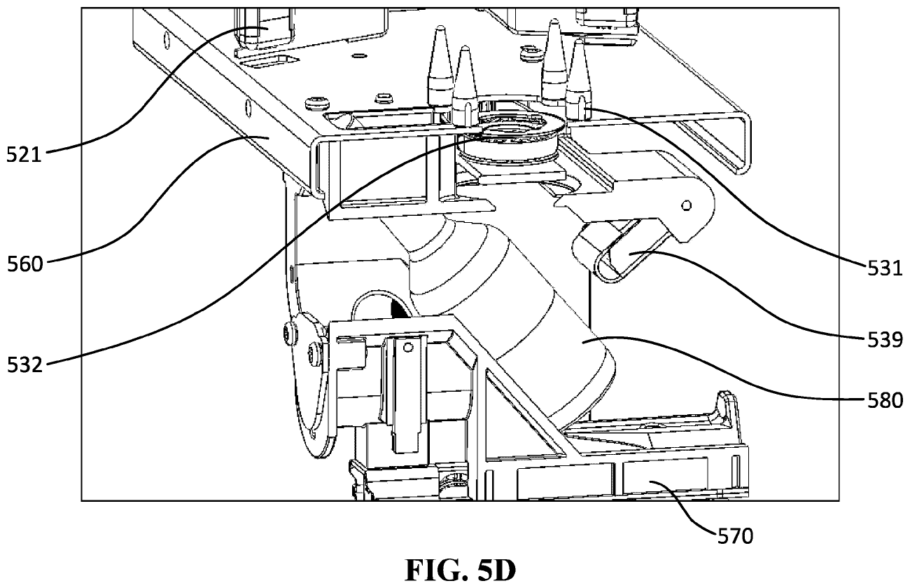

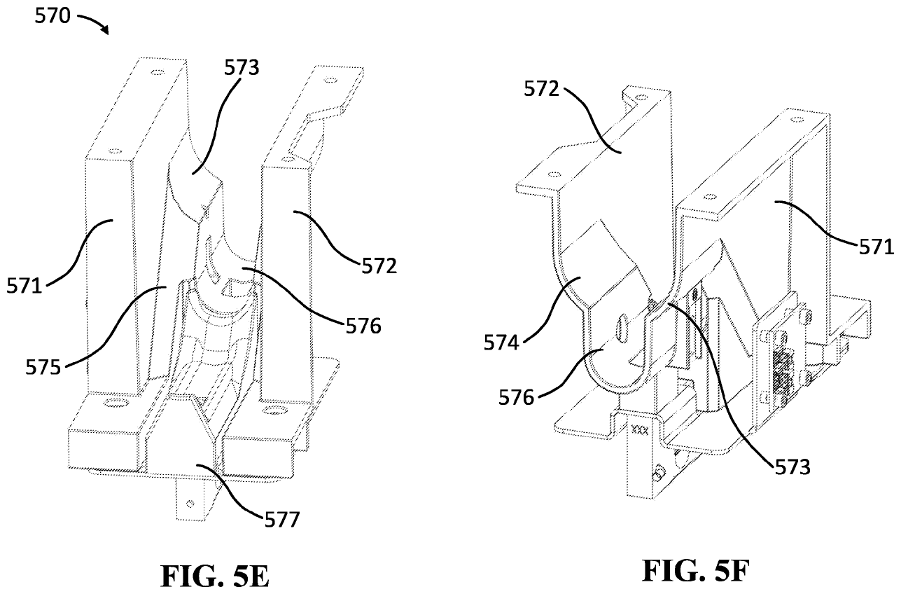

FIGS. 5A-5C provide perspective views of imaging subsystem 500. Imaging subsystem 500 may be configured to scan sample containers for label information and/or obtain image information from which the presence or absence of foam, fill level and/or other information regarding the contents of sample containers may be derived. As shown, output chutes 320 may be positioned by imaging subsystem 500, which includes camera 510, light sources 521 and 522, support structure 523, plate 524, guides 531, platform 532, drive pulley 533, belt 534, arm 535, spring 536, motor 537, opening 538, flip station 540, holding station 550, platform 560, and chute 570. Robotic subsystem 700 may deposit a sample container 580 (e.g., a blood culture bottle) on platform 532 between guides 531. Guides 531 may assist robotic subsystem 700 with centering sample container 580 on platform 532.

Camera 510 is aimed at sample container 580. Camera 510 and light sources 521 and 522 are affixed to platform 560 by support structure 523. Light sources 521 and 522 are configured to direct light towards sample container 580 as camera 510 obtains images of sample container 580. In some implementations, light source 240 may also be configured to direct light towards sample container 580 as camera 510 obtains images of sample container 580. In other implementations, these external light sources may be removed and imaging subsystem 500 may rely on one or more internal light sources of camera 510 to obtain the images of sample container 580. Platform 532 is configured to rotate as camera 510 obtains images of sample container 580. In some implementations, platform 532 may be configured such that a user can remove and replace it without using a tool. Sample container 580 may be rotated a predetermined number of degrees (e.g., 20 degrees, 30 degrees, etc.) and the images at each rotation increment may be stitched together to obtain an entire image of a label (not shown) on sample container 580. Additionally, camera 510 may obtain image information from which the presence or absence of foam, fill level and other information regarding the contents of the sample container may be derived. Camera 510 may transmit one or more of the obtained images to computer 231 and/or 370. In some implementations, one or more of the images obtained by camera 510 may be presented on display 310. These images may, for example, assist a user with the resolution of an error.

Plate 524 is set behind sample container 580. In some implementations, plate 524 may provide a static background for the images. In some implementations, plate 524 includes a label or barcode that can be used by camera 510 to determine whether a sample container is positioned on platform 532. For example, when the label or barcode is visible to camera 510, a determination can be made that a sample container is not positioned on platform 532. Similarly, when the label or barcode is not visible to camera 510, a determination can be made that a sample container is positioned on platform 532.

As best shown in FIG. 5C, in which platform 532 is illustrated in a transparent manner, the rotation of platform 532 is driven by motor 537. Drive pulley 533 is directly coupled to a shaft of motor 537. Platform 532 and drive pulley 533 are connected via belt 534. Furthermore, platform 532 and drive pulley 533 are rotatably coupled to arm 535. When motor 537 causes drive pulley 533 to rotate in a clockwise direction (from the perspective of FIG. 5C), platform 532 also rotates in a clockwise direction. However, when motor 537 causes drive pulley 533 to rotate in a counter-clockwise direction (from the perspective of FIG. 5C), arm 535 rotates in a counter-clockwise direction, causing platform 532 to move from underneath sample container 580 to a position beneath or within platform 560. As arm 535 rotates in a counter-clockwise direction, spring 536 (e.g., a torsion spring), which is directly coupled to arm 535, applies a force that opposes the force generated by motor 537. Additionally, as platform 532 is pivoted away, sample container 580 slides into chute 570 through opening 538.

Flip station 540 is configured to receive a sample container in either an upright position (see, e.g., the orientation of sample container 580) or a horizontal position (see, e.g., the orientation of sample container 760 in FIG. 7B). When a sample container is received in an upright position, it may rest on bottom surface 541 of flip station 540. However, when a sample container is received in a horizontal position, flip station flips the sample container from the horizontal position to an upright position. For example, if robotic subsystem 700 positions a sample container above flip station while holding the sample container in the manner shown in FIG. 7B and then releases the sample container, the bottom and/or sides of that sample container will slide along ramp structure 542, causing the sample container to rotate into an upright position. Since sample containers held in module 102 may be oriented horizontally, flip station 540 may be used by robotic subsystem 700 to reorient a sample container into an upright position before depositing it on platform 532 for imaging.

Holding station 550 is configured to receive a sample container in an upright position. If multiple sample containers need to be scanned by imaging subsystem 500, robotic subsystem 700 may use flip station 540 and/or holding station 550 to queue sample containers. Furthermore, if imaging subsystem 500 is unable to read the label on a sample container or if the images obtained by camera 310 indicate that the sample container has foam and/or is overfilled or underfilled, robotic subsystem 700 may use flip station 540 and/or holding station 550 to temporarily store the sample container until a user responds to a corresponding prompt on display 310. In some implementations, holding station 550 may be used to store a tool (e.g., a sample container) for calibrating camera 510.

FIG. 5D provides a cross-sectional view of the imaging subsystem 500. As shown, imaging subsystem 500 may include a rotating member 539 for preventing sample container 580 from falling out of chute 570 after being dropped from platform 532. For example, member 539 may be rotated in a counter-clockwise direction (from the perspective of FIG. 5D) before sample container 580 is dropped from platform 532. Furthermore, member 539 may be rotated in a clockwise direction (from the perspective of FIG. 5D) to make it easier for robotic subsystem 700 to retrieve sample container 580 from chute 570.

FIGS. 5E and 5F provide perspective views of chute 570. Chute 570 is similar to flip station 540, but reorients sample containers into a horizontal position instead of an upright position. As shown, chute 570 includes sidewalls 571, horizontal rails 573 and 574, sloped rail 575, recess 576, and stopper 577. After a sample container is dropped into chute 570, the bottom and/or sides of that sample container will slide along sloped rail 575 and another similarly sloped rail along sidewall 572 (not shown), causing the sample container to rotate into a horizontal position. Stopper 577 prevents a sample container from sliding out of chute 570. Since sample containers held in module 102 may be oriented horizontally, chute 570 may assists robotic subsystem 700 with reorienting an untested sample container that was received in one of compartments 330 and 340 before depositing that sample container in module 102 through one of doors 141-144.

In some implementations, robotic subsystem 700 may deliver sample containers to imaging subsystem 500 both before and after those sample containers have been incubated and measured in module 102. By scanning sample containers a second time before they are placed in compartments 330 and/or 340 for retrieval by a user and/or placed in waste receptacle 610 of waste management subsystem 600 for disposal, system 100 can provide increased confidence in the chain of custody of the sample containers. During operation, system 100 may experience conditions, such as power failure and/or unexpected user interactions, that create opportunities to lose the chain of custody for one or more sample containers.

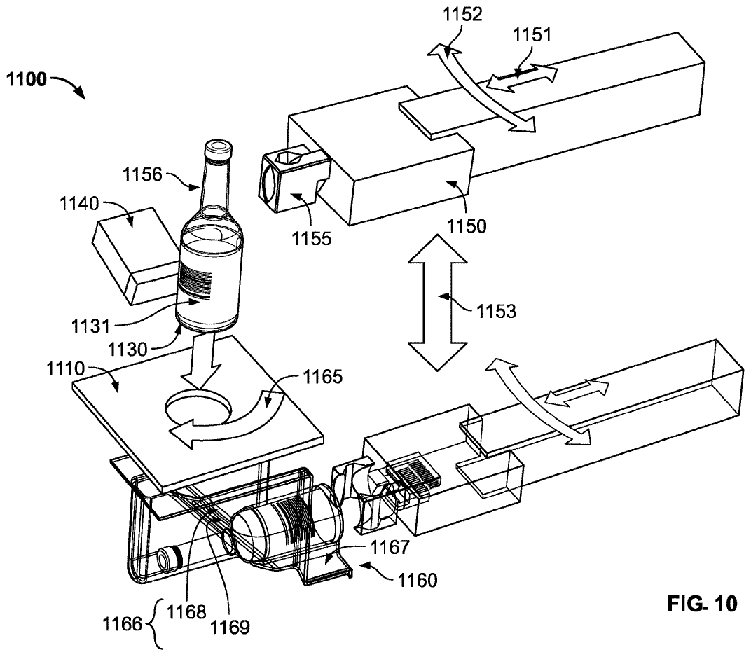

Some of the advantages of and alternatives to one or more aspects of imaging subsystem 500 are described in relation to FIGS. 10-19. For example, FIG. 10 is a schematic view of an imaging apparatus 1100. The apparatus has a platform 1110 on which the cylindrical sample container 1130 is placed. The apparatus 1100 also has a scanner 1140. A gripper arm 1150 with a clamp 1155 grips the neck 1156 of the cylindrical sample container 1130 and is used to place the cylindrical sample container 1130 onto the rotating gate 1165 of the platform 1110. The gripper arm 1150 is moveable in x (1151), theta (1152), and z (1153) so that the gripper arm 1150 may be used to place the sample container 1130 on the rotating gate 1165 in the upright position and retrieve that sample container 1130 when the sample container is lying horizontally in the chute 1160. The chute 1160 receives the cylindrical sample container in the upright position and causes the cylindrical sample container to lay in the horizontal position. Therefore chute 1160 functions as a flip station to flip the cylindrical sample container from the upright position to the horizontal position. The gripper arm 1150 is rotatable so that the clamp 1155 may grip the cylindrical sample container 1130 when the cylindrical sample container is lying horizontally. In the apparatus described in FIG. 10, an image of the label 1131 is obtained as the cylindrical sample container is rotated by the rotating gate 1165. That image is then stitched together to form a complete image of the label 1131. Stitching images together to form a larger image is well known to one skilled in the art and is not described in detail herein.

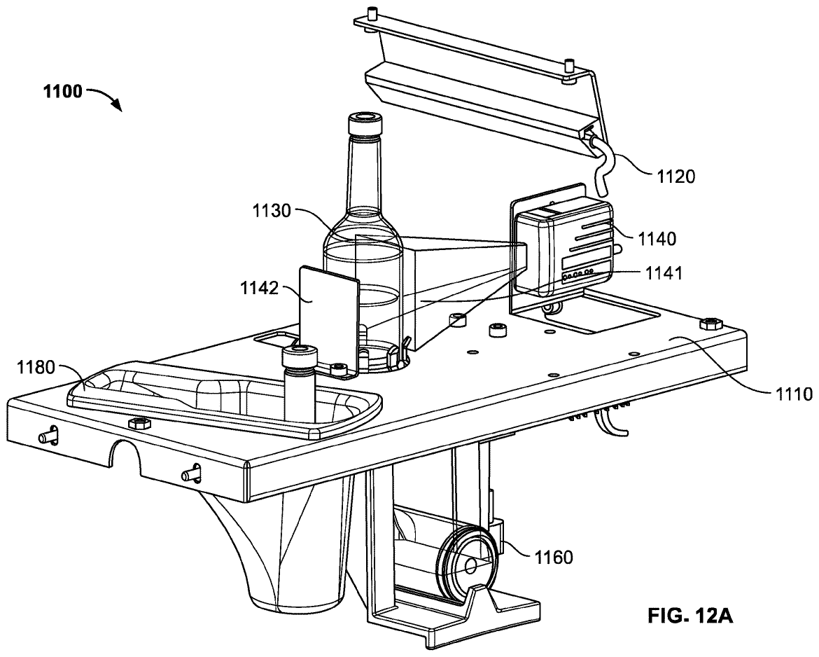

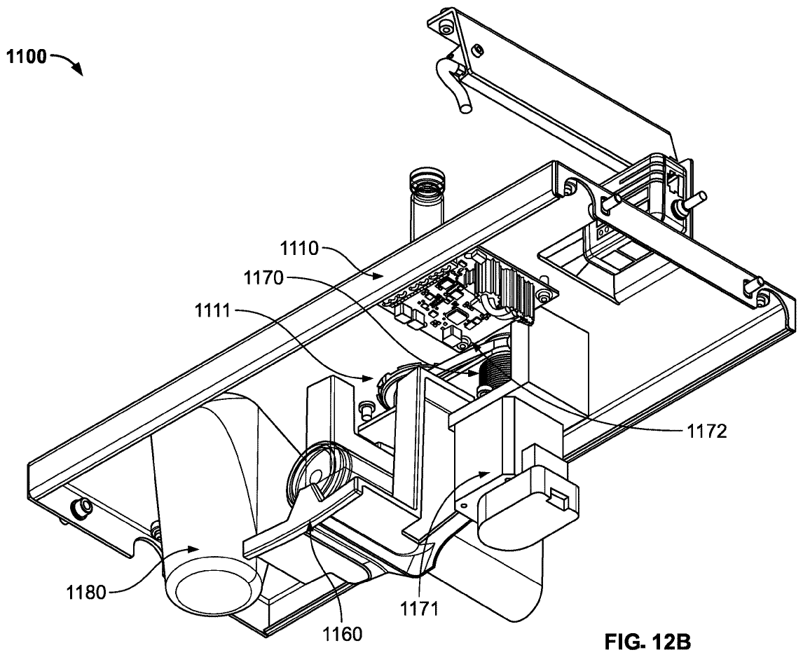

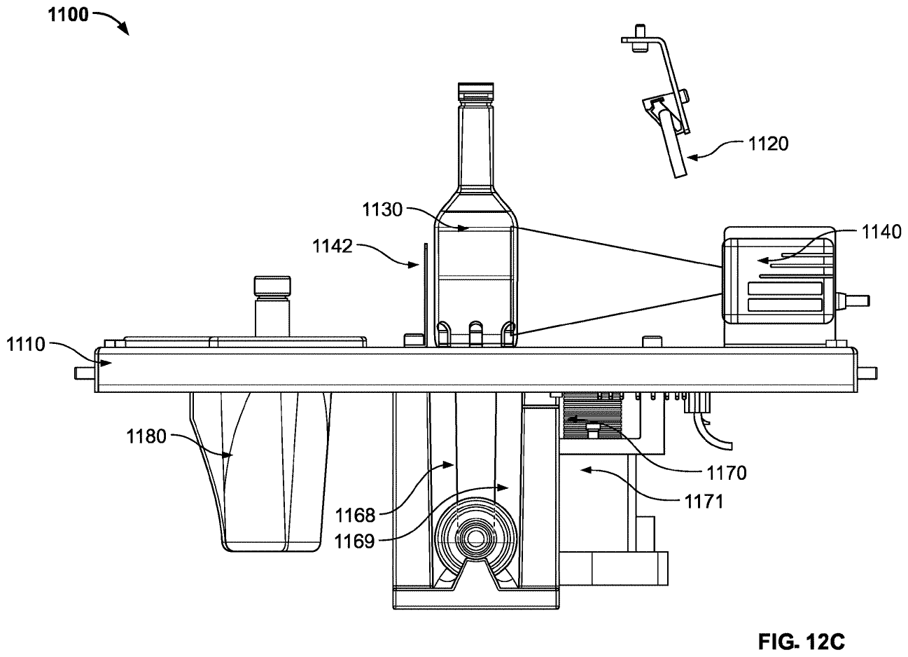

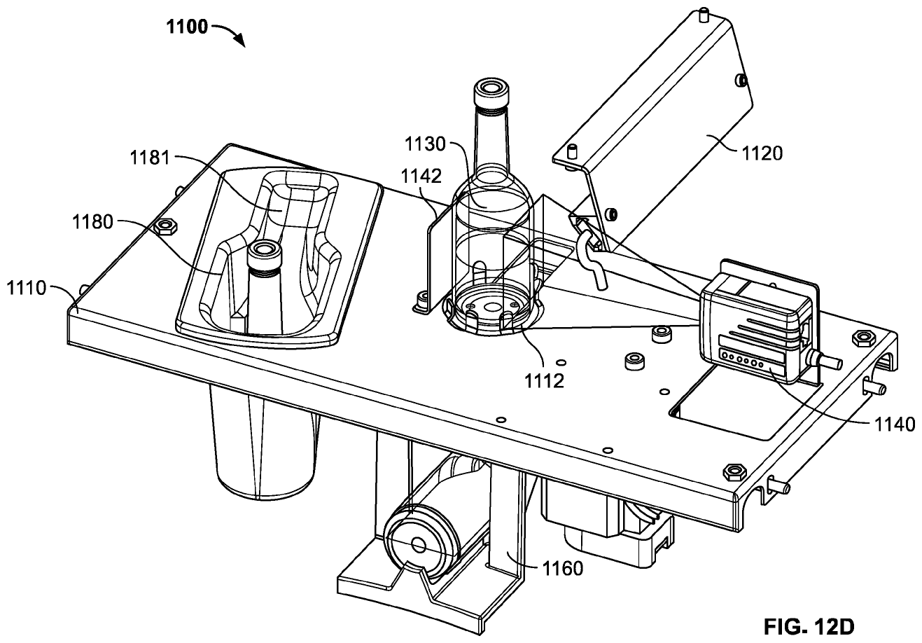

The rotating gate 1165 is rotated by a motor (not shown). Sensors or commanded steps from a theta stepper motor (not shown) inform the gripper arm 1150 to move and stop in position over the rotating plate 1165 when the clamp 1155 may release the cylindrical sample bottle 1130 on the rotating gate 1165. For imaging, the rotating platform 1110 (the rotating gate 1165 is located below the surface of the main portion of the platform 1110) rotates in one direction (either clockwise or counter clockwise). After the imaging apparatus 1100 has obtained an image of the entire label 1131 and has also obtained image information from which the presence or absence of foam, fill level and other information regarding the contents of the cylindrical sample container, imaging apparatus (e.g., camera, scanner, lights, etc.) are turned off. The rotating gate 1165 may also be actuated out of alignment with the chute 1160. When the rotating gate 1165 is aligned with the chute 1160, the cylindrical sample container does not slip through the chute when the bottle is placed on the rotating gate 1165 for imaging. The complete image may be formed by taking several images before and after rotating the bottle by about 45 degrees, for example, and then stitching those images together to provide an image of the complete bottle. After imaging, the rotating platform 1110 rotates in the opposite direction until the gate 1165 is actuated out of alignment with the opening for the chute 1160. This allows the cylindrical sample container 1130 to slip through the opening the chute 1160, which has a ramp 1166 and a platform 1167. The cylindrical sample container 1130 eases down ramp 1166 and comes to rest horizontally on platform 1167, from where it is retrieved by the clamp 1155 of gripper arm 1150. In this regard the ramp 1166 has tracks 1168, 1169 which are spaced apart so that, as the cylindrical sample container 1130 eases down the ramp 1166, the neck of the cylindrical sample container 1130 fits between tracks 1168, 1169, allowing the cylindrical sample container 1130 to lie flat. Tracks 1168 and 1169 are more readily observed in FIG. 12C.

Not shown are a calibration plate that is disposed on the end of the platform 1110 opposite the scanner 1140. The calibration plate may be used to calibrate the scanner 1140 to ensure that, when the cylindrical sample container 1130 is placed on the rotating gate 1165, it will be in the correct field of view for the scanner. The rotating gate 1165 is configured to provide a stable surface on which to set the cylindrical sample container 1130 for imaging. Since sterilizing the cylindrical sample containers prior to use may introduce deformities or irregularities in the bottom surface of the cylindrical sample containers 1130, the rotating gate 1165 may be provided with recessed portion that will allow the perimeter of the bottom of the cylindrical sample container to seat securely on the rotating gate 1165 yet provides a clearance between the interior of the bottom surface of the cylindrical container and the surface of the cylindrical sample container 1130 so that any surface deformities do not cause the cylindrical sample container to seat in an unstable manner.

Alternatives structures to the rotating gate include rubber drive wheels that are adject the cylindrical sample container or rotating grippers such as those used to screw on or screw off caps automatically. If such rotating mechanisms are used, the system is provided with a trap door or other mechanism to allow the cylindrical sample container to advance into the chute when the imaging is complete.

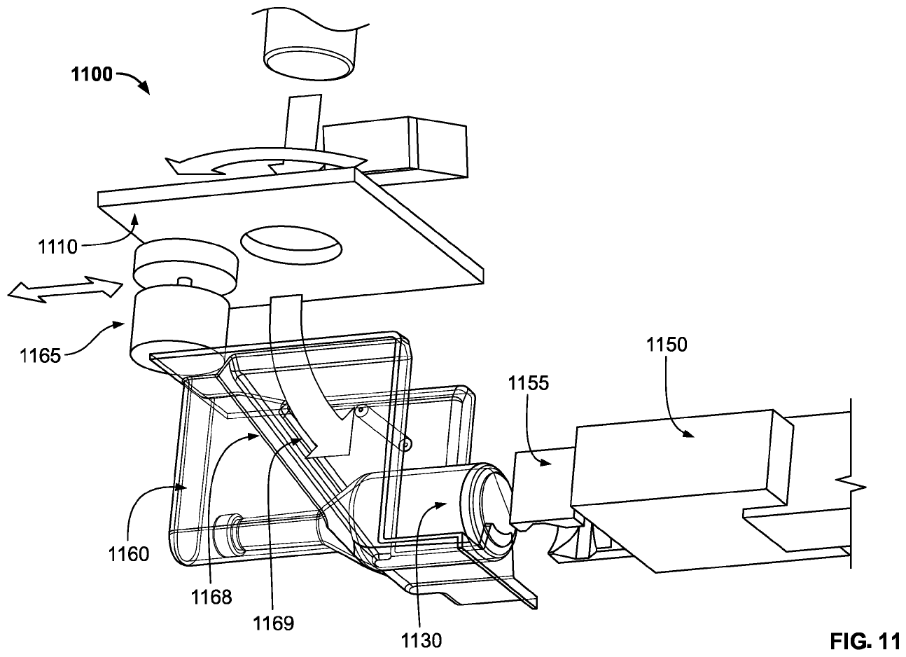

FIG. 11 is a bottom view of the imaging apparatus 1100 of FIG. 10. In FIG. 11 the rotating gate 1165 is illustrated out of alignment with chute 1160. After the cylindrical sample container 1130 has traveled down chute 1160, it rests in a horizontal position with its neck disposed between tracks 1168, 1169. The clamp 1155 of the gripper arm 1150 rotates to grip the bottom of cylindrical sample container 1130 to remove it from the chute.

FIGS. 12A-12D illustrate an alternative implementation system 1100 of FIG. 10 in which platform 1110 has a rotating platform 1111 mounted beneath it. FIG. 12A is a perspective view from above the system 1100. FIG. 12B is an upward perspective view of the system 1100. FIG. 12C is a side view of the system 1100. FIG. 12D is a downward perspective view of the system 1100. The rotating platform 1111 is driven by shaft (obscured by coil spring 1170) that is rotated by a motor 1171 from which the shaft inside coil spring 1170 extends. A belt 1172 couples the shaft to the rotating platform 1111, causing the rotation of the rotating platform 1111. After the camera 1140 obtains an image of the cylindrical sample container 1130, the rotation of the shaft inside coil spring 1170 is reversed, and, when the rotating platform rotates in the opposite direction, the rotating platform 1111 is pivoted away through use of a one-way rotation clutch, allowing the cylindrical sample container 1130 to slide into chute 1160 through opening 1112 in platform 1100. The coil spring 1170 opposes the rotating platform's motion causing it to swing back into place when the motor rotates the platform back to the original direction. While the cylindrical sample container 1130 is being rotated, it may be illuminated by a light source 1120.

The implementation of FIGS. 12A-12D has a holding station 1180, for the cylindrical sample container 1130. The holding station 1180 has a ramp structure 1181 so that the cylindrical sample container 1130 will sit in an upright position as long as it placed bottom first into the holding station 1180. The robotic arm 1150 is used to bring the cylindrical sample container 1130 into the holding station 1180. The robotic arm 1150 also moves the cylindrical sample container 1130 to the imaging location 1141 and places it therein, and retrieves the cylindrical sample container 1130 from the chute 1160. A plate 1142 is set behind the imaging location 1141. In some implementations, plate 1142 provides a static background for the image. In some implementations, plate 1142 includes a label or barcode that can be used by scanner 1140 to determine whether a sample container is positioned at the imaging location 1141. For example, when the label or barcode is visible to scanner 1140, a determination can be made that a sample container is not positioned at the imaging location 1141. Similarly, when the label or barcode is not visible to scanner 1140, a determination can be made that a sample container is positioned at the imaging location 1141. The cylindrical sample container 1130 is rotated a predetermined number of degrees (e.g., 20 degrees, 30 degrees, etc.) and the images at each rotation increment are then stitched together to obtain an entire image of the label.

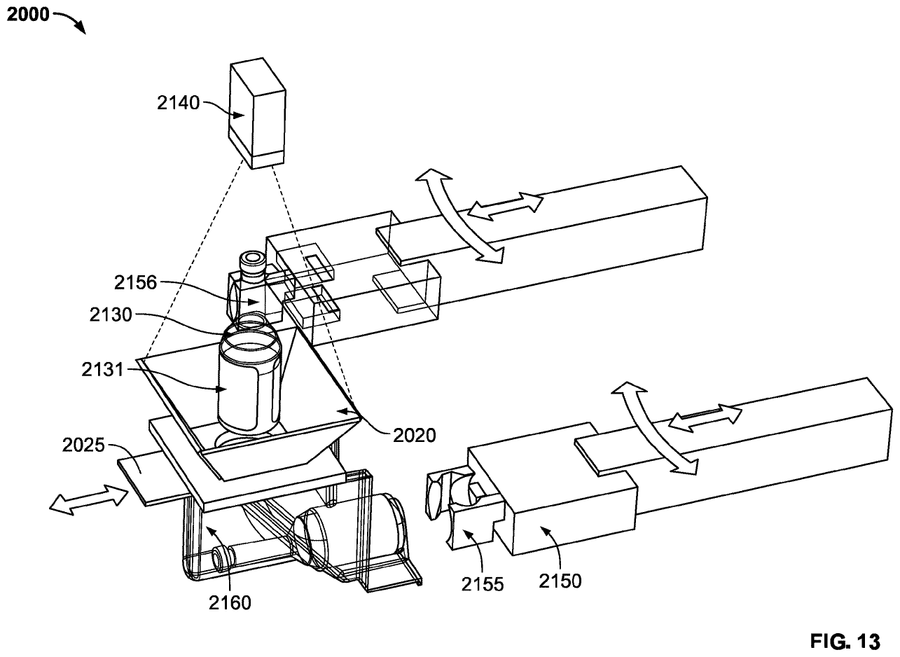

FIG. 13 is an alternative implementation of FIG. 10, but one in which the cylindrical sample container is not rotated. In this implementation, the system 2000 has a pyramidal or conical mirror 2020 for which the complete image can be formed by taking several images. The system has a trap door 2025 that slides horizontally. When the trap door 2025 is advanced inward, it holds the cylindrical sample container 2130 in place for imaging by scanner 2140. The image that is captured is of the entire label 2131. A gripper arm 2150 with a clamp 2155 grips the neck 2156 of the cylindrical sample container 2130 and is used to place the cylindrical sample container 2130 into the pyramidal mirror 2020 for imaging. The gripper arm 2150 is moveable in x, theta, and z so that the gripper arm 2150 may be used to place the cylindrical sample container 2130 in the pyramidal mirror 2020 in the upright position and retrieve the cylindrical sample container 2130 when the cylindrical sample container is lying horizontally in the chute 2160. When the trap door 2025 is advanced outward, the cylindrical sample container 2130 falls through the chute 2160 and is removed by the gripper arm 2150. Alternative implementations deploy other types of doors for allowing the cylindrical sample container to descend into the chute 2160. Examples of suitable alternative doors include drop away doors, sliding doors or a retracting pin.

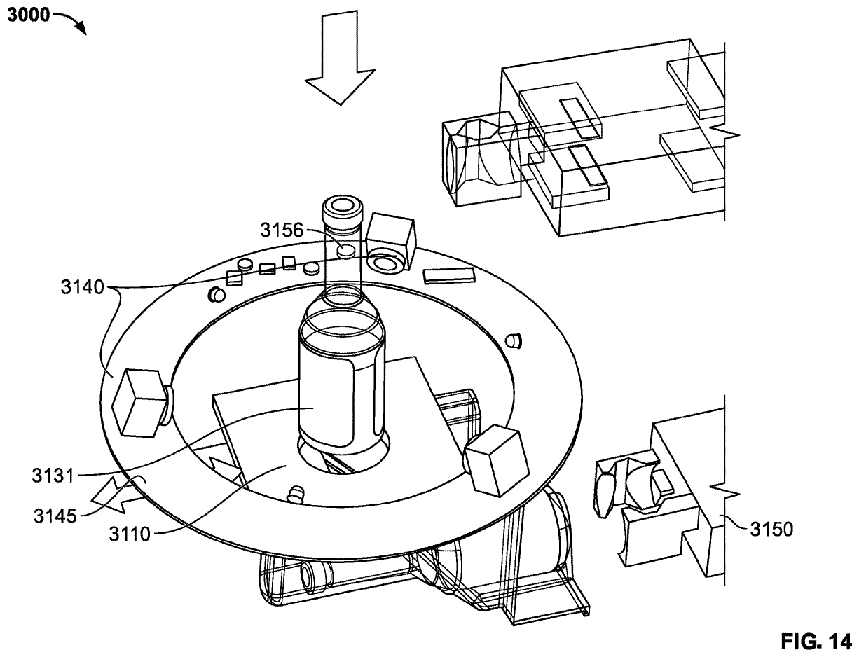

FIG. 14 illustrates an alternative system 3000 in which multiple cameras 3140 are used to obtain images of the label 3131 on the cylindrical sample container 3130. Obtaining an image with multiple cameras is a well-known technique for assembling a “flat” image from a cylindrical object, as each image is only a segment of the curved object. Stitching such images together is also well-known and not described in detail herein. The system 3000 has a platform 3110 with a trap door 3025. The trap door 3025 is closed and the cylindrical sample container is held on the platform 3110 for imaging. As illustrated, the cameras 3140 are mounted on a ring-shaped printed circuit board 3145. As described above, a gripper arm 3150 is used to grip the neck 3156 of the cylindrical sample container 3130 and place it in the imaging apparatus. After imaging, the trap door 3025 is actuated and the cylindrical sample container 3130 falls through the chute 3160 and is removed by the gripper arm 3150.

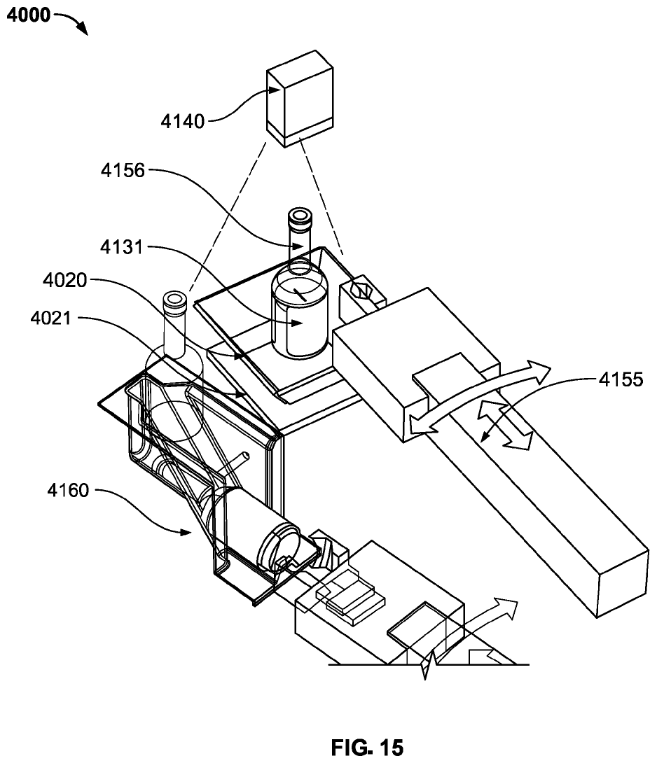

FIG. 15 illustrates a system 4000 that does not have a trap door. In this implementation, the gripper arm 4150 is used to place and remove the cylindrical sample container 4130 from the pyramidal mirror 4020 which has no opening in its base 4021. The scanner 4140 is used to obtain a single image of the entire expanse of label 4131. After imaging, in this implementation, should the user seek to have the cylindrical sample container gripped by the base rather than the neck, the gripper arm 4150 will remove the cylindrical sample container 4130 from the pyramidal mirror 4020 and place it in the chute 4160 wherein it will slide to a horizontal position as described above, after which the gripper arm 4150 will remove the cylindrical sample container from the chute 4160 by gripping the base of the cylindrical sample container 4130.

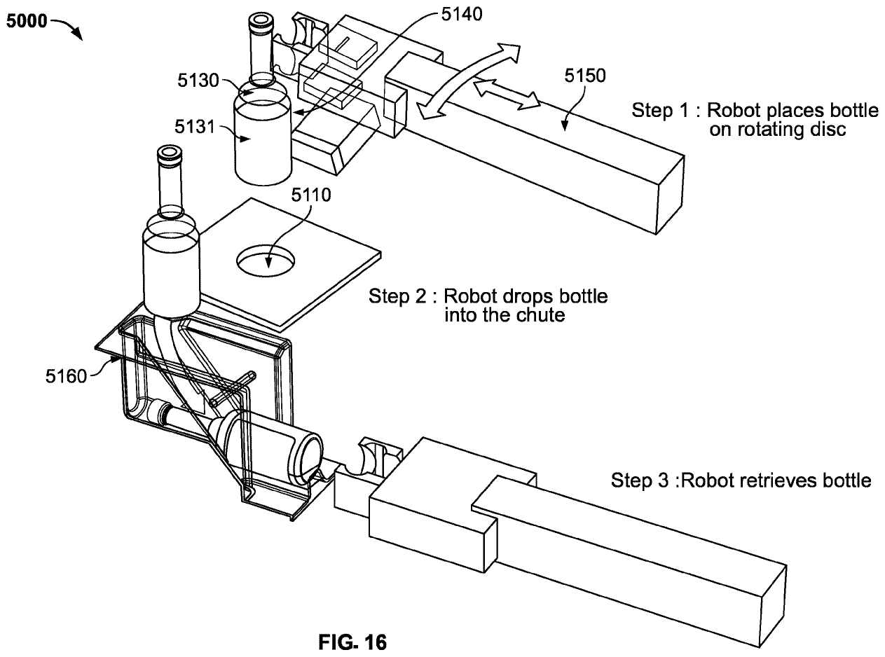

FIG. 16 is a system 5000 such as is illustrated in FIG. 10 but wherein the gripper arm 5150 moves the cylindrical sample container 5130 to the imaging position and to the chute 5160 that flips the cylindrical sample container from the upright to the horizontal position. The gripper arm 5150 has the scanner 5130 mounted thereon. Once the gripper arm 5150 places the cylindrical sample container 5130 on to the rotating platform 5110, the gripper arm then advances to align the scanner 5140 with the cylindrical sample container 5130 to obtain an image of the label 5131 as the rotating platform 5110 rotates the cylindrical sample container 5130. After the image of the cylindrical sample container is obtained, the gripper arm 5150 then moves the cylindrical sample container to the chute 5160. When placed in the chute 5160 the cylindrical sample container flips from the vertical position in which it is placed to the horizontal position, where it is retrieved by the gripper arm 5150 by grasping the bottom of the cylindrical sample container 5130.

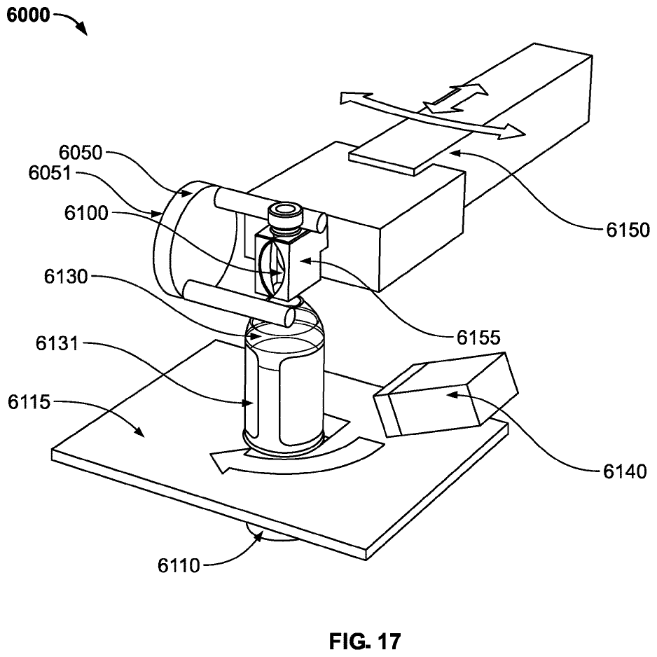

FIG. 17 is a system 6000 that does not use a chute to rotate the cylindrical sample container from the upright position to the horizontal position. System 6000 deploys a tilting gripper 6050 that grips the neck 6100 of the cylindrical sample container 6130. The system 6000 uses the rotating platform 6110, placed underneath platform 6115 to ensure that images of the entire label 6131 are captured by the scanner 6140. After an image of the cylindrical sample container 6130 is captured by the scanner 6140, the end effector 6155 of the gripper arm 6150 rotates and sets the tilting gripper 6050 such that the flat surface 6051 of the tilting gripper rests on the platform 6115. The gripper arm then releases the neck 6100 of the cylindrical sample container 6130. The cylindrical sample container is then held in the horizontal position by the tilting gripper 6050 resting on platform 6115. The gripper arm 6150 then rotates and advances end effector 6155 in position to grasp the bottom of the cylindrical sample container 6130, which is being held in the horizontal position. The end effector 6155 then grasps the bottom of the cylindrical sample container 6130 and conveys the cylindrical sample container 6130 away from the platform 6100. The tilting gripper is not conveyed away with the cylindrical sample container 6130.

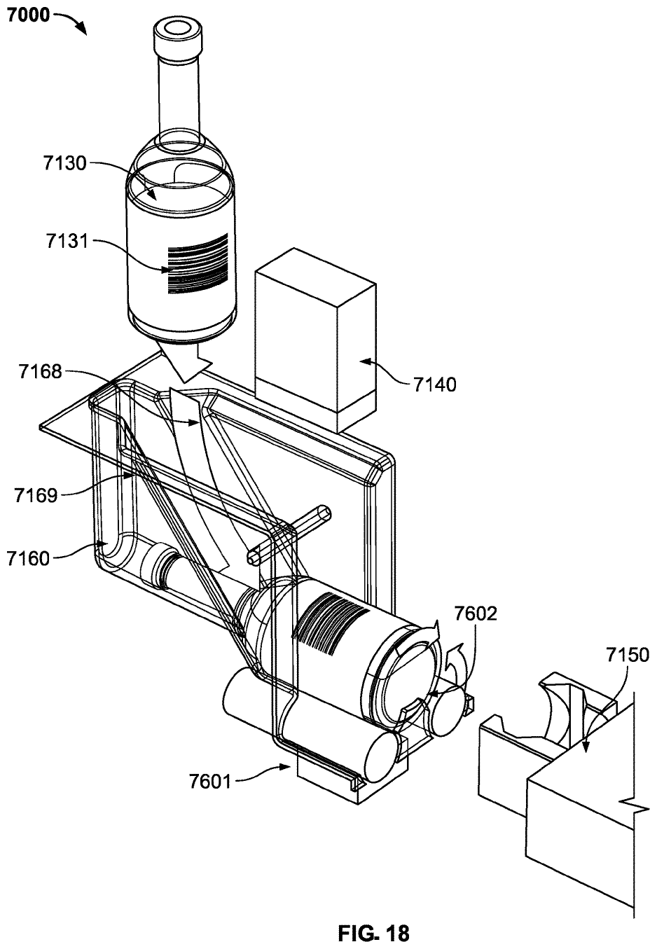

FIG. 18 illustrates a system 7000 that deploys the chute 7160 to rotate the cylindrical sample container 7130 as it lays horizontally in the chute 7160. As described above, the gripper arm 7150 holds the cylindrical sample container in a vertical orientation. The gripper arm 7150 places the cylindrical sample container 7130 in the chute where it slides down ramp 7166 along tracks 7168 and 7169. The neck 7100 of the cylindrical sample container 7130 fits between tracks 7168, 7169. The chute 7160 has rollers 7601 and 7602. The rollers may be used to cause the cylindrical sample container 7130 to rotate. With the scanner 7140 placed over the rotating cylindrical sample container, an image of the label 7131 is obtained. However, when the cylindrical sample container 7130 is in the horizontal position, the meniscus of the inoculated culture in the neck 7100 of the cylindrical sample container 7130 cannot be observed. Therefore, in this system, the volume of the sample (e.g., blood) added to the sample cannot be ascertained by observing the cylindrical sample container in its horizontal position. After an image of the label 7131 on the cylindrical sample container is obtained, the gripper arm 7150 grabs the bottom of the cylindrical sample container 7130 to remove it from chute 7160.

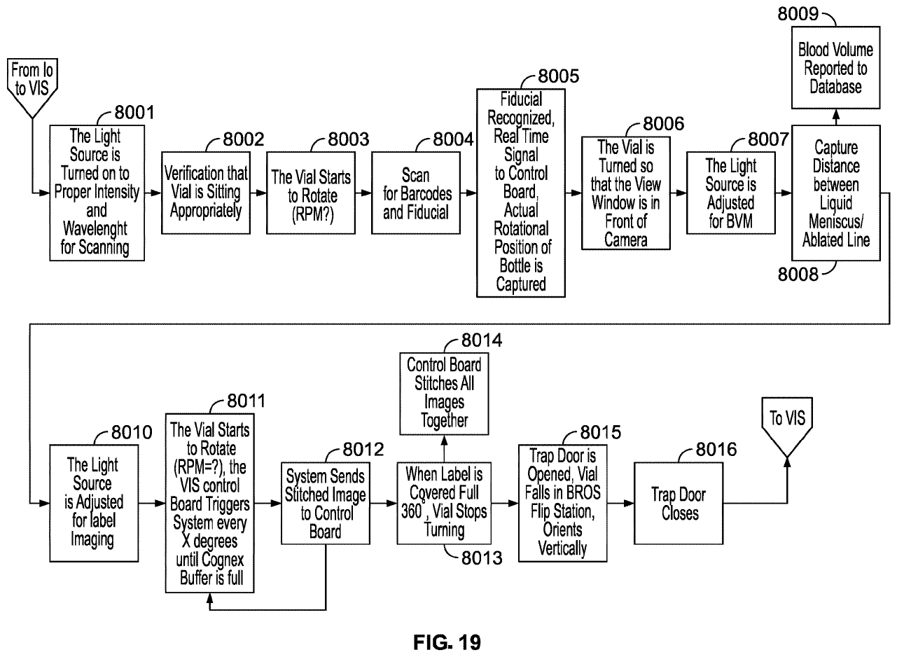

FIG. 19 is a flow chart for the positioning of the cylindrical sample container for imaging. In step 8001, the light source for the scanner is turned on and tuned so the correct intensity and wavelength for scanning. This step is controlled by software. In step 8002, sensors verify that the cylindrical sample container is in the correct position. In those implementations where the cylindrical sample container is rotated, rotation of the bottle is commenced in step 8003. The scanner then scans the bar code and any fiducial marks on the cylindrical sample container in step 8004. In step 8005, when the fiducial is recognized, the position of the cylindrical sample container is captured by the system. In step 8006, the cylindrical sample container is rotated so that the view window (i.e., a portion of the cylindrical sample container that is not covered by the label) is disposed in front of the scanner/camera to determine the liquid level in the cylindrical sample container. In step 8007, the light source is adjusted for blood volume measurements (BVM). In step 8008 the system captures a distance between the liquid meniscus in the cylindrical sample container and a line etched on the cylindrical sample container (for volume determination). The ablation line is etched at a custom height on the bottle during manufacturing denoting the intended fill level of the patient blood at bedside. Typical fill is 8-10 ml for adults and 3 ml for pediatrics using special pediatric sample containers. Each media type has a published expected fill volume, which is used in computing amount of user overfill or underfill. The volume of the patient blood in the sample container is determined using the difference in height between the blood line and the ablation line. By knowing the volume characteristics of the cylindrical sample container, the amount of patient blood fill is calculated.

In step 8009 the blood volume is reported to the data base. In step 8010 the light source is adjusted (e.g., from blue to red or white) to obtain an image of the label. In step 8011, the cylindrical sample container is rotated at the set speed. An image is captured after a preset number of degrees (e.g., 20 degrees) of rotation until a full series of images of the entire label is obtained. In some implementations, the triggers for capturing these images are provided directly by a motor (e.g., a stepper motor controller) without the use of an encoder. In step 8012, the images are stitched together to form a full image of the label. The stitched image information is fed back to the rotation controller, which continues to rotate the cylindrical sample container until the buffer that receives the image information is full. In step 8013, when the cylindrical sample container has rotated a full 360 degrees, the rotation is stopped. In step 8014 all of the label images are stitched together. In those systems where a trap door is provided to release the cylindrical sample container into the chute, the trap door is opened in step 8015. In step 8016, the trap door closes. In those systems where the chute flips from vertical to horizontal, the cylindrical sample container is retrieved in the horizontal position.

### Waste Management Subsystem

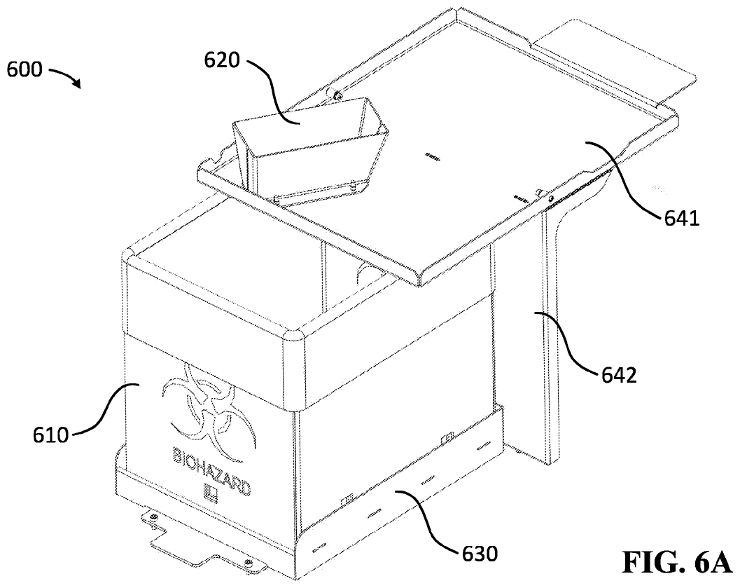

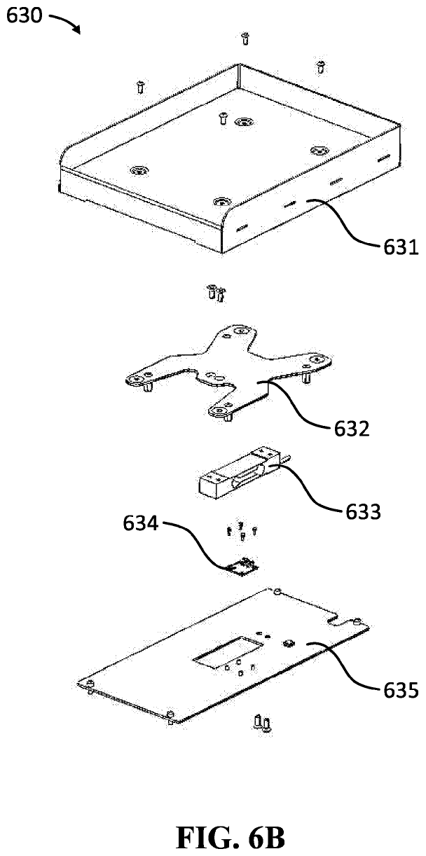

FIG. 6A provides a perspective view of waste management subsystem 600. As shown, waste management subsystem 600 includes waste receptacle 610, chute 620, waste receptacle holder 630, and support structures 641 and 642. A user can access waste receptacle 610 by opening door 110 (see FIGS. 1A-1F) and pulling waste receptacle 610 over a lip of waste receptacle holder 630. Chute 620 is positioned above waste receptacle 610 and supported by support structures 641 and 642. During operation, robotic subsystem 700 may drop negative sample containers into waste receptacle 610 through chute 620. This alleviates the user from the workload associated with negative sample containers, which is usually ˜90% of the sample containers subjected to testing for the presence of biologically active agents. In some implementations, waste management subsystem 600 may include one or more additional chutes (not shown) into which robotic subsystem 700 may drop negative sample containers into waste receptacle 610.

In some implementations, waste management subsystem 600 may include one or more sensors, such as touch sensors, optical sensors, and/or ultrasonic sensors, for monitoring system conditions. For example, FIG. 6B provides an exploded view of an implementation of waste receptacle holder 630. As shown, waste receptacle holder 630 includes base 631, support structures 632 and 635, load cell 633, and controller 634. In some implementations, controller 634 may include one or more processors, one or more application specific integrated circuits (ASICs), and/or other similar components. Controller 634 may also include a memory medium, such as a hard-drive, memory card, ROM, RAM, DVD, CD-ROM, write-capable, and/or read-only memory, that is capable of storing information. Load cell 633 may, for example, be used for detecting whether waste receptacle 610 is full. As another example, load cell 633 may be used for detecting whether waste receptacle 610 is positioned on base 631 of waste receptacle holder 630. As yet another example, load cell 633 may be used for detecting the addition of a single sample container to waste receptacle 610. Such information may, for example, be used to verify that a sample container was successfully released by robotic subsystem 700. Measurements from any of the sensors in waste management subsystem 600 may be transmitted to computer 231 and/or 370, which may then cause a corresponding alert to appear on display 310. For example, controller 634 may transmit measurements from load cell 633 to computer 370, which may then cause a corresponding alert to appear on display 310. In some implementations, one or more illumination lights may be positioned on or near door 110 to communicate similar information to a user. In some such implementations, these indicator lights may be different colors, change colors, and/or flash.

### Robotic Subsystem

FIGS. 7A-7H provide perspective views of robotic subsystem 700 and/or one or more of its components. Robotic subsystem 700 may be configured to transfer sample containers to and/or from module 102, output chutes 320, compartments 330 and 340, imaging subsystem 500, and/or waste management subsystem 600. Robotic subsystem 700 may also be configured to automatically distribute and/or redistribute sample containers around the circumference of one or more drums in module 102 to distribute the sample containers as desired. As shown in FIG. 7A, robotic subsystem 700 includes z-axis robot 710, theta-axis robot 720, r-axis robot 730, gripper assembly 740, and controller 751. Z-axis robot 710 is configured to raise and lower theta-axis robot 720, r-axis robot 730, and gripper assembly 740. Theta-axis robot 720 is configured to rotate r-axis robot 730 and gripper assembly 740. R-axis robot 730 is configured to move gripper assembly 740 forwards and backwards. Gripper assembly 740 is configured to grab and release sample containers (e.g., blood culture bottles).

As shown, z-axis robot 710 includes rail 711, counterweight housing 712, pulleys 713, motor 714, and counterweight 715. Z-axis robot 710 employs a counterweight system to improve the speed of robotic subsystem 700 and to improve overall throughput of sample containers. The counterweight system includes counterweight housing 712, pulleys 713, and counterweight 715. One or more cables (not shown) may be coupled to both theta-axis robot 720 and counterweight 715, which is positioned within counterweight housing 712. The one or more cables may extend from theta-axis robot 720, through pulleys 713, and into counterweight housing 712. In some implementations, one or more redundant cables may be used for safety reasons should the primary cable break. The counterweight system may facilitate the use of components with reduced ratings, weight, cost, and/or size, such as a lower torque motor (e.g., motor 714) and/or a rail (e.g., rail 711) that has a lower moment load rating on a carriage. Thus, the counterweight system may help reduce the overall weight, cost, and/or size of robotic subsystem 700.

FIG. 7B provides a perspective view of robotic subsystem 700 without z-axis robot 710. As shown, robotic subsystem 700 may include controller 752, cable carrier 753, and camera 770. In some implementations, controller 751 may be communicatively coupled to r-axis robot 730 and gripper assembly 740, and controller 752 may be communicatively coupled to theta-axis robot 720. For example, controllers 751 and 752 may transmit commands and/or receive measurement data from theta-axis robot 720, r-axis robot 730, and/or gripper assembly 740. In some implementations, controllers 751 and 752 may communicate with one or more controllers (e.g., controllers 751, 752, and/or 754) and/or computers (e.g., computer 231 and/or 370) through one or more electrical conduits or wireless communications. In some implementations, controllers 751 and 752 may include one or more processors, one or more application specific integrated circuits (ASICs), and/or other similar components. Controllers 751 and 752 may also include a memory medium, such as a hard-drive, memory card, ROM, RAM, DVD, CD-ROM, write-capable, and/or read-only memory, that is capable of storing information. In some implementations, cable carrier 753 may house one or more electrical conduits coupled to r-axis robot 730 and/or gripper assembly 740. Much like controllers 751 and 752, controller 754 (see FIG. 2E) may be communicatively coupled to z-axis robot 710. For example, controller 754 may transmit commands and/or receive measurement data from z-axis robot 710 through one or more electrical conduits or wireless communications.

In some implementations, camera 770 may be used to verify movements of robotic subsystem 700. For example, camera 770 may be used to verify that robotic subsystem 700 has successfully grabbed or released a sample container. As another example, camera 770 may be used to verify that gripper assembly 740 is positioned correctly relative to one or more components of module 101, such as output chutes 320, compartments 330 and 340, imaging subsystem 500, or waste management subsystem 600. In some implementations, camera 770 may enable a user to more easily view the movements of robotic subsystem 700. For example, one or more images captured by camera 770 may be shown on display 310. In some implementations, camera 770 may communicate with one or more controllers (e.g., controllers 751, 752, and/or 754) and/or computers (e.g., computer 231 and/or 370) through one or more electrical conduits or wireless communications. In some implementations, camera 770 may be removed from robotic subsystem 700. In some implementations, module 101 may include one or more sensors, such as touch sensors, optical sensors, and/or ultrasonic sensors, to verify the position of robotic subsystem 700 and/or one or more of its components. In some implementations, the electrical current and/or power drawn by one or more motors of robotic subsystem 700 (e.g., motors 714, 721, 732, and/or 741) may be used to verify whether one or more movements of robotic subsystem 700 have been started or completed.

FIG. 7C provides a perspective view of r-axis robot 730 and gripper assembly 740. Cover 731 (see FIG. 7B) has been removed to reveal a system of pulleys in r-axis robot 730. As shown, r-axis robot 730 includes motor 732, belt 733, drive pulley 734, idler pulleys 735, arms 736 and 737, and clamp 738. Drive pulley 734 and idler pulleys 735 are connected via belt 733. Drive pulley 734 is also directly coupled to a shaft of motor 732. When motor 732 causes drive pulley 734 to rotate, idler pulleys 735 will also rotate. Additionally, clamp 738, which is coupled to belt 733, will move forwards or backwards when drive pulley 734 rotates. Since clamp 738 is also coupled to arm 737, which is slidingly engaged with arm 736, the forwards and backwards movement of clamp 738 will also cause arm 737 to move forwards and backwards. The forward and backward movement of arm 737 will also cause gripper assembly 740, which is coupled to arm 737, to move forwards and backwards. As shown, drive pulley 734 and some of idler pulleys 735 are toothed. The remaining idler pulleys 735 are smooth. However, in other implementations one or more of these pulleys can be modified to be smooth or toothed.

As shown, r-axis robot 730 also includes belt tensioner 780, which includes idler pulley 781, arm 782, coupling 783, and screw 784. Idler pulley 781 contacts belt 733 and may be used to apply tension to belt 733 and to prevent it from slipping. As shown, idler pulley 781 is smooth, but in other implementations idler pulley 781 may be toothed. Idler pulley 781 is rotatably coupled to arm 782. In some implementations, arm 782 is rotatably coupled to cover 731 and/or another component of r-axis robot 730 via coupling 783. Arm 782 may rotate about an axis that extends through coupling 783. Screw 784 can be tightened to apply a force to arm 782, which causes arm 782 to rotate in a clockwise direction (from the perspective of FIG. 7C) and force idler pulley 781 to apply additional tension to belt 733. In some implementations, belt tensioner 780 may include one or more springs (not shown) to automatically set the desired tension.

As shown in FIG. 7C, motor 732 is advantageously positioned in the middle of the stroke of r-axis robot 730. Arm 737 can be moved backwards past motor 732, and arm 737 can be moved forwards past motor 732. This is accomplished by using a plurality of idler pulleys 735, as opposed to a single idler pulley. Most commercially available actuators place the motor at one end of the stroke. This may conveniently enable the actuator to only use two pulleys--a drive pully on one end that is coupled to the shaft of a motor and an idler pully at the opposite end of the stroke. This is a cost-effective solution, but results in a longer overall length for the actuator due to the motor size. Put another way, this type of design requires space at the end of the actuator for the motor. In module 101, there is minimal space behind r-axis robot 730. Therefore, it is advantageous to be able to reduce the overall length of r-axis robot 730 by using a plurality of idler pulleys 735, as shown in FIG. 7C.

Various modifications can be made to r-axis robot 730. For example, in some implementations, motor 732 may be moved to another position between the ends of the stroke of r-axis robot 730. As another example, in some implementations, r-axis robot 730 may include more or less of idler pulleys 735. As yet another example, in some implementations, belt 733, drive pulley 734, and idler pulleys 735 may be rotated 90 degrees to be parallel with a plane of the top surfaces of arms 736 and 737. As yet another example, in some implementations, different belt types and sizes, pulley sizes, and/or belt routings could also be used.

FIGS. 7D and 7E provide perspective views of gripper assembly 740 and portions of r-axis robot 730. As shown, r-axis robot 730 includes track 748, which may be used to couple arms 736 and 737. For example, track 748 of arm 737 may be coupled to rail 749 of arm 736 (see FIG. 7C). Furthermore, gripper assembly 740 includes motor 741, grippers 742 and 743, and recesses 744 and 745. During operation, motor 741 may be used to move grippers 742 and 743 closer together or farther apart. These motions enable robotic subsystem 700 to grab and release sample containers. Furthermore, as shown, robotic subsystem 700 can grab sample container 760 (e.g., a blood culture bottle) when it is oriented vertically (see FIG. 7D) and when it is oriented horizontally (see FIG. 7E). When sample container 760 is in an upright position, recesses 744 and 745 may be used to grasp a neck of sample container 760. When sample container 760 is in a horizontal position, the curved shape of grippers 742 and 743 may be used to grasp a bottom end of sample container 760.

In some implementations, grippers 742 and 743 may be forced together by one or more springs (not shown). In such implementations, motor 741 generates an opposing force to move grippers 742 and 743 farther apart. In some implementations, the one or more springs may be positioned within a common housing with motor 741. In the event of a power failure, the one or more springs may prevent robotic subsystem 700 from dropping a sample container. For example, the force generated by the one or more springs may be sufficient for robotic subsystem 700 to continue gripping a sample container even when there is no power. In some implementations, robotic subsystem 700 may include a back-up power supply (e.g., back-up power supply 234). In addition to helping prevent robotic subsystem 700 from dropping a sample container, a back-up power supply may also help maintain the chain of custody for sample containers. Furthermore, in some implementations, a back-up power supply may enable robotic subsystem 700 to finish delivering a sample container to a destination location during a power outage. For example, if robotic subsystem 700 was in the middle of delivering a sample container to imaging subsystem 500, the back-up power supply may be used to complete that delivery.

As shown in FIGS. 7A-7E, gripper assembly 740 is advantageously separated from arm 736 of r-axis robot 730 by arm 737. In other implementations, gripper assembly 740 may also be slidingly coupled to arm 737. However, in such implementations, it may not be possible to fully retract gripper assembly 740 to a position beneath arm 736 while it is holding a sample container in an upright position. As best seen in FIG. 7C, a crimp ring, a septum, and a portion of a neck of a sample container may extend vertically past the tops of grippers 742 and 743. These portions of the sample container may crash into arm 736 in implementations where gripper assembly 740 is directly coupled to arm 737. In the implementation illustrated in FIGS. 7A-7E, this is avoided by creating a space between the tops of grippers 742 and 743 and arm 736 that can accommodate a crimp ring, a septum, and/or a portion of a neck of a sample container. Advantageously, this space is created without also increasing the size (e.g., height) of gripper assembly 740. Thus, this implementation provides a cost-effective way to be able to fully retract gripper assembly 740 while it is holding a sample container in an upright position.

As shown in FIGS. 7A-7E, the orientation of belt 733 and rail 749 may also advantageously contribute to the ability of robotic subsystem 700 to fully retract gripper assembly 740 while it is holding a sample container in an upright position. More specifically, a space beneath arm 736, which is also between belt 733 and rail 749, can accommodate a crimp ring, a septum, and/or a portion of a neck of a sample container while it is being held by gripper assembly 740 in an upright position. Advantageously, this space is created without also increasing the size (e.g., height) of gripper assembly 740. Thus, the orientation of belt 733 and rail 749 provides a cost-effective way to be able to fully retract gripper assembly 740 while it is holding a sample container in an upright position.

FIGS. 7F-7H provide perspective views of theta-axis robot 720. As shown, theta-axis robot 720 includes motor 721, belt 722, idler pulley 723, drive pulley 724, platform 725, and coupling 726. Drive pulley 724 and idler pulley 723 are connected via belt 722. Drive pulley 724 is also directly coupled to a shaft of motor 721. When motor 721 causes drive pulley 724 to rotate, idler pulley 723 will also rotate. Since idler pulley 723 is also coupled to r-axis robot 730, the rotation of drive pulley 724 will also cause r-axis robot 730 to rotate. Theta-axis robot 720 may be slidingly coupled to rail 711 of z-axis robot 710 via coupling 726.

As shown, theta-axis robot 720 also includes belt tensioner 790, which includes idler pulley 791, plate 792, recess 793, screws 794, and openings 795. Idler pulley 791 contacts belt 722 and may be used to apply tension to belt 722 and to prevent it from slipping. Idler pulley 791 is rotatably coupled to plate 792. Plate 792 is positioned within a recess 793 of platform 725. Plate 792 is coupled to screws 794, which extend through openings 795 of platform 725. When screws 794 are loose, plate 792 can slide upwards or downwards (from the perspective of FIG. 7G). As plate 792 slides downwards, it causes idler pulley 791 to apply additional tension to belt 722. When screws 794 are tightened, plate 792 is prevented from sliding upwards or downwards. In some implementations, belt tensioner 790 may include one or more springs (not shown) to automatically set the desired tension.

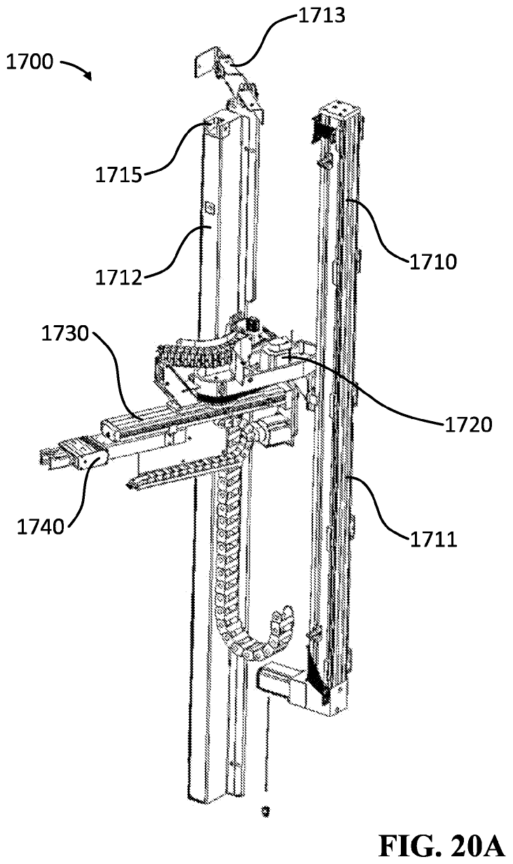

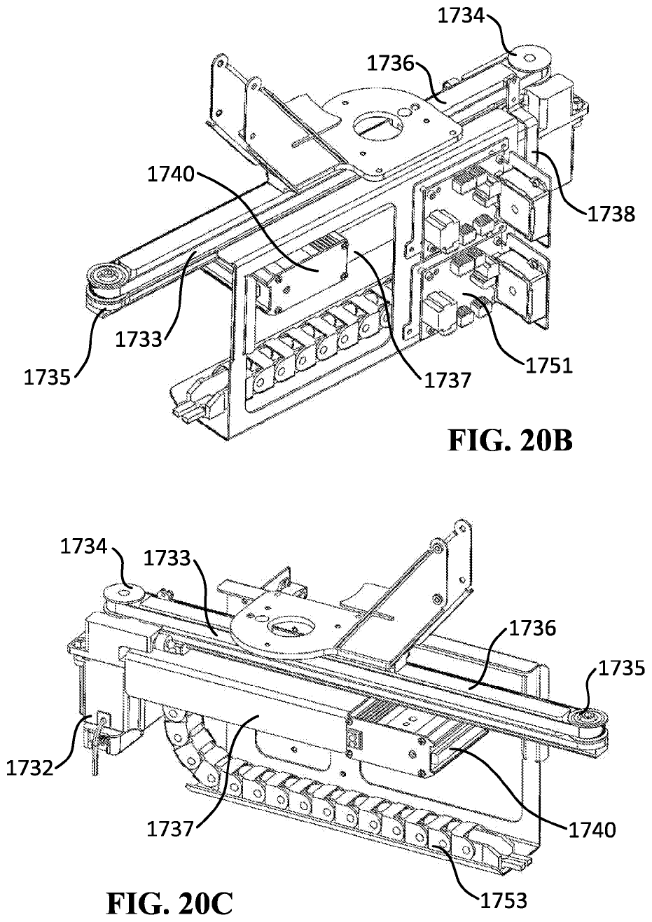

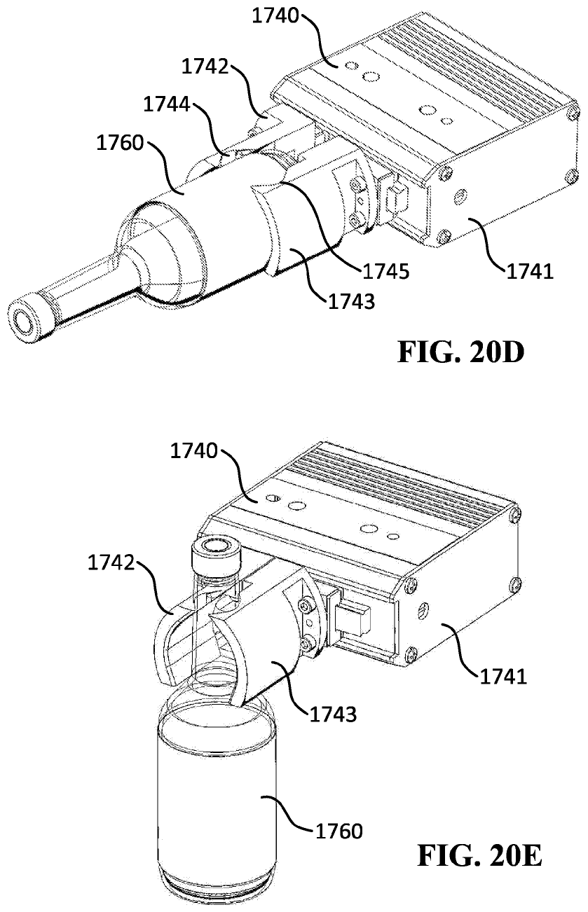

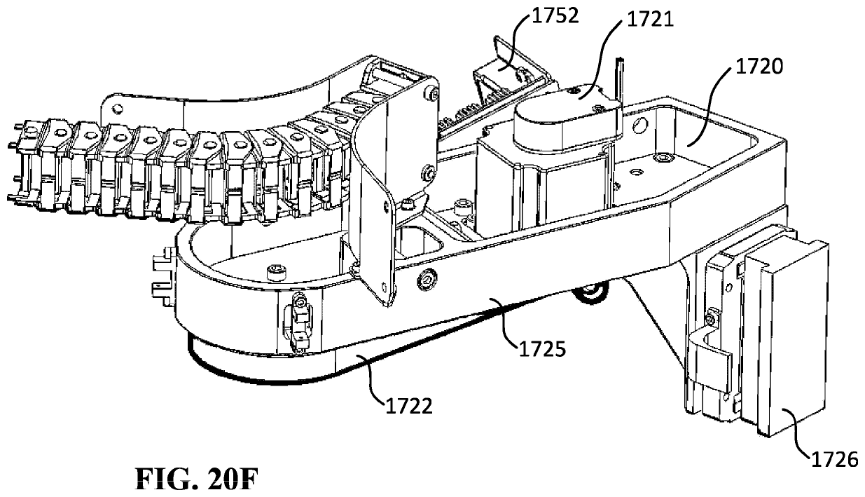

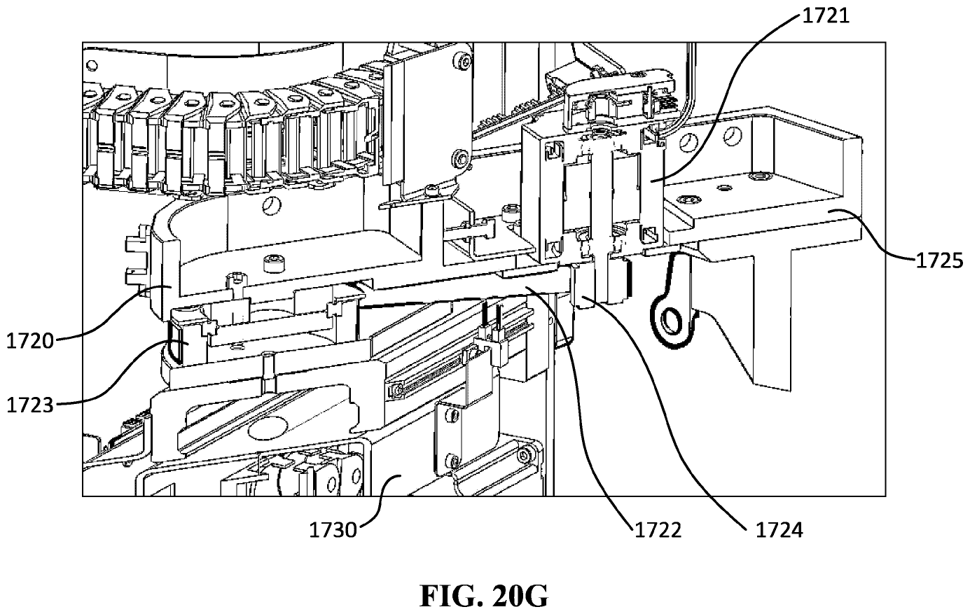

FIGS. 20A-20G illustrate another implementation of a robotic subsystem that may be compared to robotic subsystem 700. As shown in FIG. 20A, robotic subsystem 1700 includes z-axis robot 1710, theta-axis robot 1720, r-axis robot 1730, and gripper assembly 1740. Z-axis robot 1710 is configured to raise and lower theta-axis robot 1720, r-axis robot 1730, and gripper assembly 1740. Theta-axis robot 1720 is configured to rotate r-axis robot 1730 and gripper assembly 1740. R-axis robot 1730 is configured to move gripper assembly 1740 forwards and backwards. Gripper assembly 1740 is configured to grab and release sample containers (e.g., blood culture bottles).

As shown, z-axis robot 1710 includes rail 1711, counterweight housing 1712, pulleys 1713, and counterweight 1715. Z-axis robot 1710 employs a counterweight system to improve the speed of robotic subsystem 1700 and to improve overall throughput of sample containers. The counterweight system includes counterweight housing 1712, pulleys 1713, and counterweight 1715. One or more cables (not shown) may be coupled to both theta-axis robot 1720 and counterweight 1715, which is positioned within counterweight housing 1712. The one or more cables may extend from theta-axis robot 1720, through pulleys 1713, and into counterweight housing 1712. In some implementations, one or more redundant cables may be used for safety reasons should the primary cable break. The counterweight system may facilitate the use of components with reduced ratings, weight, cost, and/or size, such as a lower torque motor and/or a rail (e.g., rail 1711) that has a lower moment load rating on a carriage. Thus, the counterweight system may help reduce the overall weight, cost, and/or size of robotic subsystem 1700.

FIGS. 20B and 20C provide perspective views of r-axis robot 1730 and gripper assembly 1740. As shown, r-axis robot 1730 includes motor 1732, belt 1733, drive pulley 1734, idler pulley 1735, arms 1736 and 1737, and clamp 1738. Drive pulley 1734 and idler pulley 1735 are connected via belt 1733. Drive pulley 1734 is also directly coupled to a shaft of motor 1732. When motor 1732 causes drive pulley 1734 to rotate, idler pulley 1735 will also rotate. Additionally, clamp 1738, which is coupled to belt 1733, will move forwards or backwards when drive pulley 1734 rotates. Since clamp 1738 is also coupled to arm 1737, which is slidingly engaged with arm 1736, the forwards and backwards movement of clamp 1738 will also cause arm 1737 to move forwards and backwards. The forward and backward movement of arm 1737 will also cause gripper assembly 1740, which is coupled to arm 1737, to move forwards and backwards. As shown, drive pulley 1734 and idler pulley 1735 are toothed, but in other implementations drive pulley 1734 and idler pulley 1735 may be toothed.

Various modifications can be made to r-axis robot 1730. For example, in some implementations, r-axis robot 1730 may include more or less of idler pulleys. As yet another example, in some implementations, belt 1733, drive pulley 1734, and idler pulley 1735 may be rotated 90 degrees to be parallel with a plane of the top surfaces of arms 1736 and 1737. As yet another example, in some implementations, different belt types and sizes, pulley sizes, and/or belt routings could also be used. As yet another example, in some implementations, r-axis robot 1730 could be modified to have a carriage driven by a ball-screw and nut, rather than a linear actuator driven by an internal belt drive with pulleys at each end.

FIGS. 20D and 20E provide perspective views of gripper assembly 1740. As shown, gripper assembly 1740 includes motor 1741, grippers 1742 and 1743, and recesses 1744 and 1745. During operation, motor 1741 may be used to move grippers 1742 and 1743 closer together or farther apart. These motions enable robotic subsystem 1700 to grab and release sample containers. Furthermore, as shown, robotic subsystem 1700 can grab sample container 1760 (e.g., a blood culture bottle) when it is oriented vertically (see FIG. 20E) and when it is oriented horizontally (see FIG. 20D). When sample container 1760 is in an upright position, recesses 1744 and 1745 may be used to grasp a neck of sample container 1760. When sample container 1760 is in a horizontal position, the curved shape of grippers 1742 and 1743 may be used to grasp a bottom end of sample container 1760.

In some implementations, grippers 1742 and 1743 may be forced together by one or more springs (not shown). In such implementations, motor 1741 generates an opposing force to move grippers 1742 and 1743 farther apart. In some implementations, the one or more springs may be positioned within a common housing with motor 1741. In the event of a power failure, the one or more springs may prevent robotic subsystem 1700 from dropping a sample container. For example, the force generated by the one or more springs may be sufficient for robotic subsystem 1700 to continue gripping a sample container even when there is no power. In some implementations, robotic subsystem 1700 may include a back-up power supply (e.g., back-up power supply 234). In addition to helping prevent robotic subsystem 1700 from dropping a sample container, a back-up power supply may also help maintain the chain of custody for sample containers.

FIG. 20F provides a perspective view of theta-axis robot 1720 and FIG. 20G provides a cross-sectional view of portions of theta-axis robot 1720 and r-axis robot 1730. As shown, theta-axis robot 1720 includes motor 1721, belt 1722, idler pulley 1723, drive pulley 1724, platform 1725, and coupling 1726. Idler pulley 1723 comprises a large diameter (˜3.5″) thin section bearing containing steel ball bearings with a machined gear ring sandwiched around it. Drive pulley 1724 and idler pulley 1723 are connected via belt 1722. Drive pulley 1724 is also directly coupled to a shaft of motor 1721. When motor 1721 causes drive pulley 1724 to rotate, idler pulley 1723 will also rotate. Since idler pulley 1723 is also coupled to r-axis robot 1730, the rotation of drive pulley 1724 will also cause r-axis robot 1730 to rotate. Theta-axis robot 1720 may be slidingly coupled to rail 1711 of z-axis robot 1710 via coupling 1726.

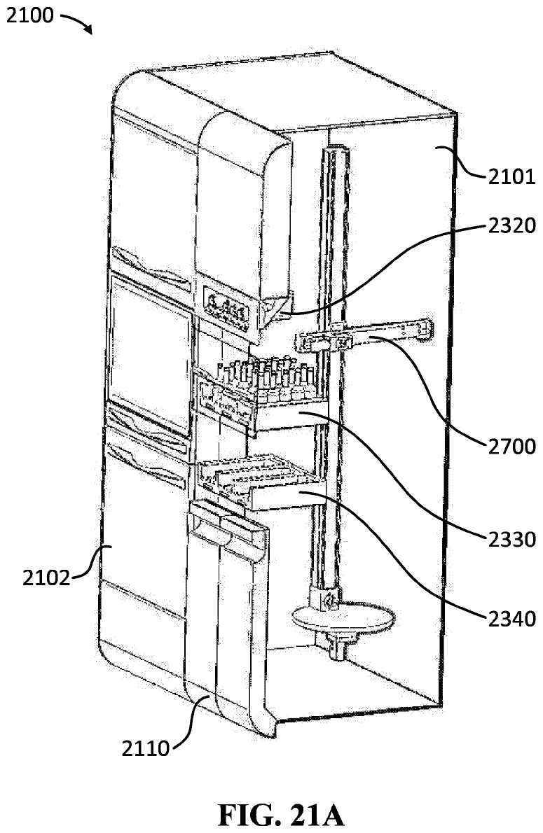

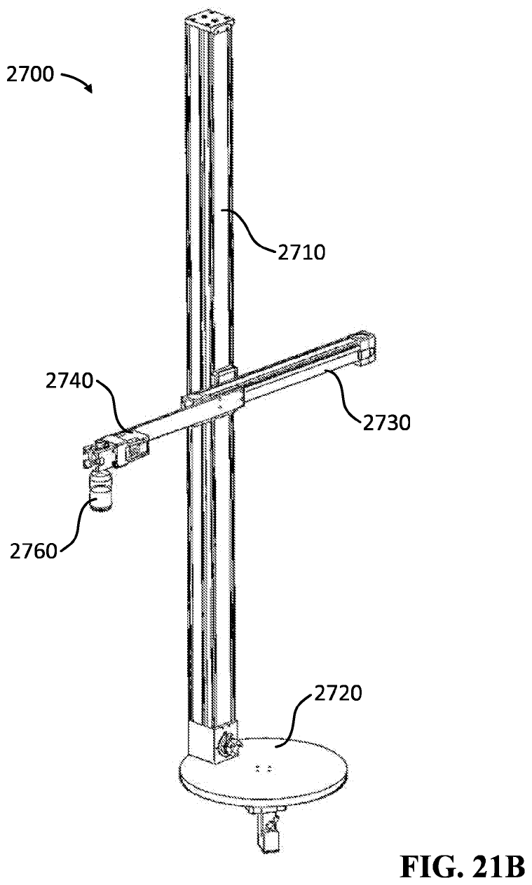

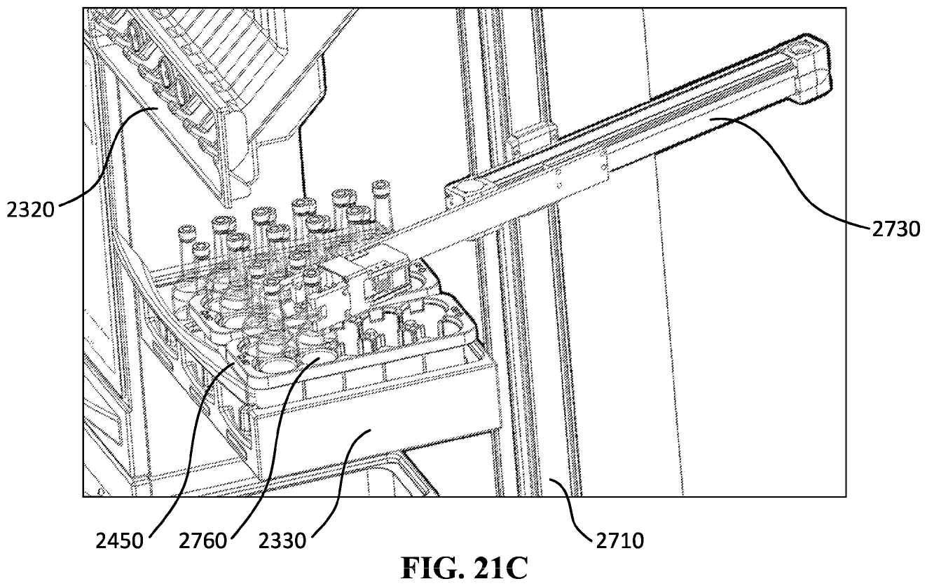

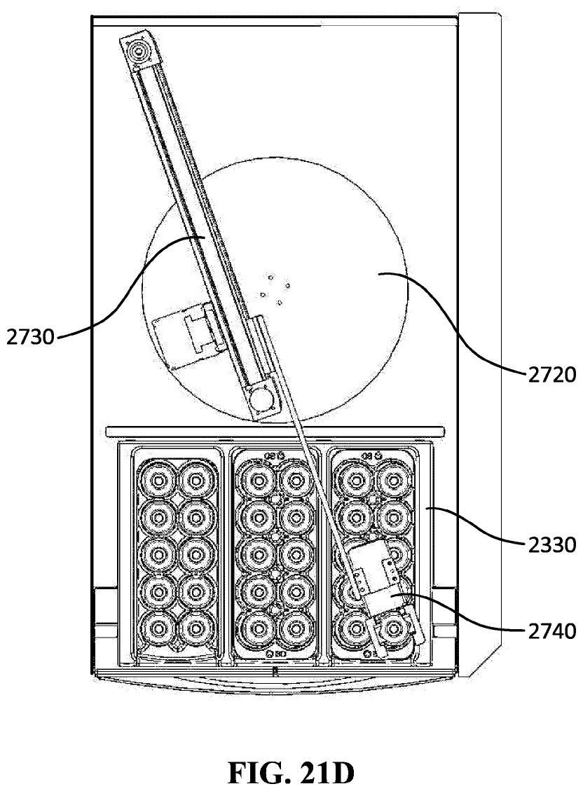

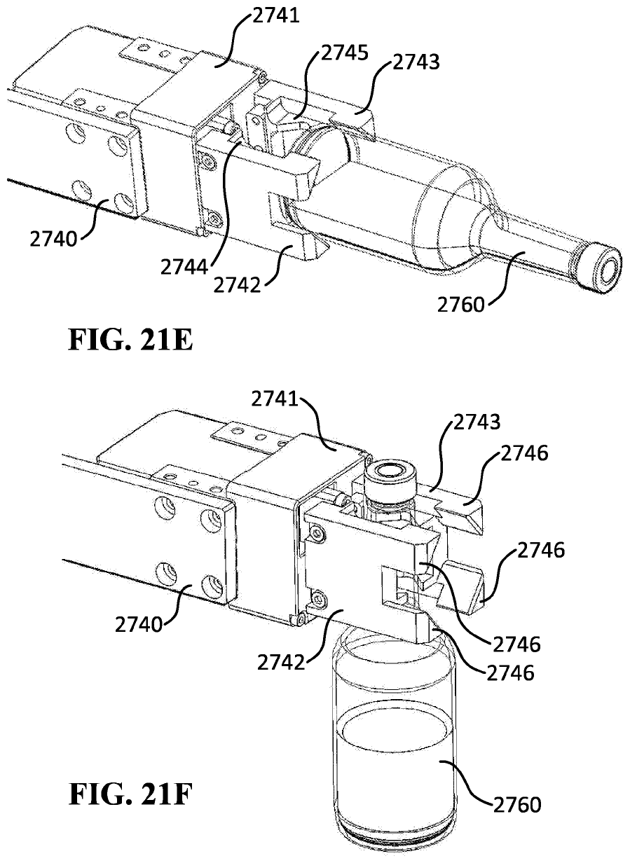

FIGS. 21A-21F illustrate another implementation of a robotic subsystem that may be compared to robotic subsystems 700 and 1700. As shown in FIG. 21A, robotic subsystem 2700 may be incorporated into an automated system 2100 for processing a plurality of sample containers (e.g., blood culture bottles) that includes modules 2101 and 2102. Module 2101 is a sample handling module that is configured to receive sample containers, scan sample containers, transfer sample containers to and from module 2102, dispose of sample containers that test negative, and provide sample containers that test positive at an output. As shown, module 2101 includes output chutes 2320, compartments 2330 and 2340, and door 2110, which may provide access to a waste receptacle. Module 2102 is an incubation and measurement module that is configured to determine whether the sample containers are contaminated with or infected by microorganisms.

As shown in FIG. 21B, robotic subsystem 2700 includes z-axis robot 2710, theta-axis robot 2720, r-axis robot 2730, and gripper assembly 2740. Z-axis robot 2710 is configured to raise and lower r-axis robot 2730 and gripper assembly 2740. Theta-axis robot 2720 is configured to rotate z-axis robot 2710, r-axis robot 2730, and gripper assembly 2740. R-axis robot 2730 is configured to move gripper assembly 2740 forwards and backwards. Gripper assembly 2740 is configured to grab and release sample containers (e.g., blood culture bottles). As shown in FIGS. 21C and 21D, robotic subsystem 2700 can retrieve a sample container 2760 from a rack 2450 in compartment 2330.

FIGS. 21E and 21F provide perspective views of gripper assembly 2740. As shown, gripper assembly 2740 includes motor 2741, grippers 2742 and 2743, recesses 2744 and 2745, and fingers 2746. During operation, motor 2741 may be used to move grippers 2742 and 2743 closer together or farther apart. These motions enable robotic subsystem 2700 to grab and release sample containers. Furthermore, as shown, robotic subsystem 2700 can grab sample container 2760 (e.g., a blood culture bottle) when it is oriented vertically (see FIG. 21F) and when it is oriented horizontally (see FIG. 21E). When sample container 2760 is in an upright position, recesses 2744 and 2745 may be used to grasp a neck of sample container 2760. When sample container 2760 is in a horizontal position, fingers 2746 may be used to grasp a bottom end of sample container 2760. In some implementations, gripper assembly 2740 may include additional fingers.

In some implementations, grippers 2742 and 2743 may be forced together by one or more springs (not shown). In such implementations, motor 2741 generates an opposing force to move grippers 2742 and 2743 farther apart. In some implementations, the one or more springs may be positioned within a common housing with motor 2741. In the event of a power failure, the one or more springs may prevent robotic subsystem 2700 from dropping a sample container. For example, the force generated by the one or more springs may be sufficient for robotic subsystem 2700 to continue gripping a sample container even when there is no power. In some implementations, robotic subsystem 2700 may include a back-up power supply (e.g., back-up power supply 234). In addition to helping prevent robotic subsystem 2700 from dropping a sample container, a back-up power supply may also help maintain the chain of custody for sample containers.

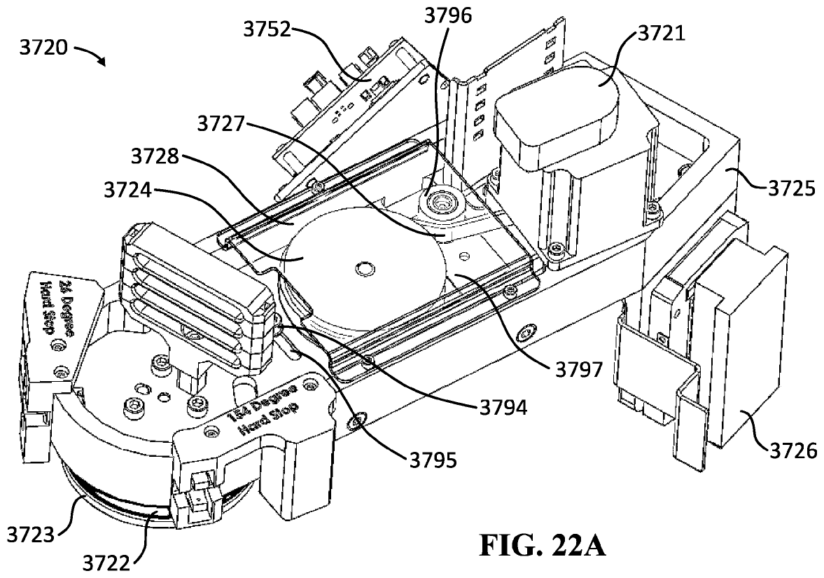

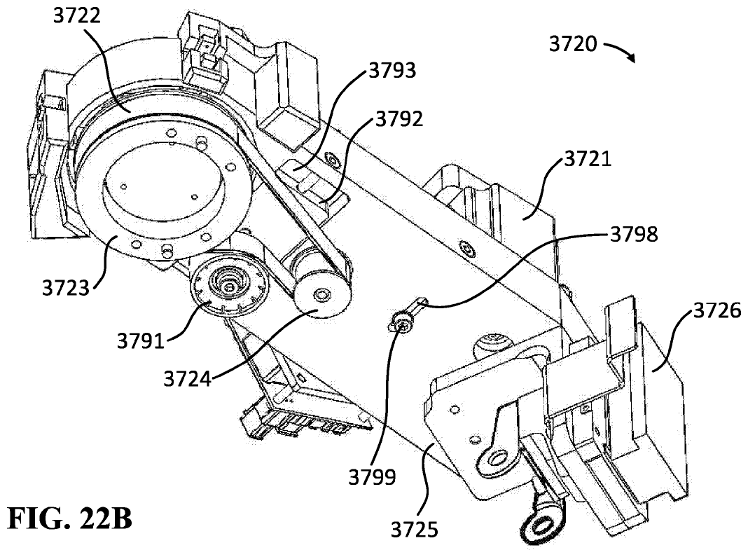

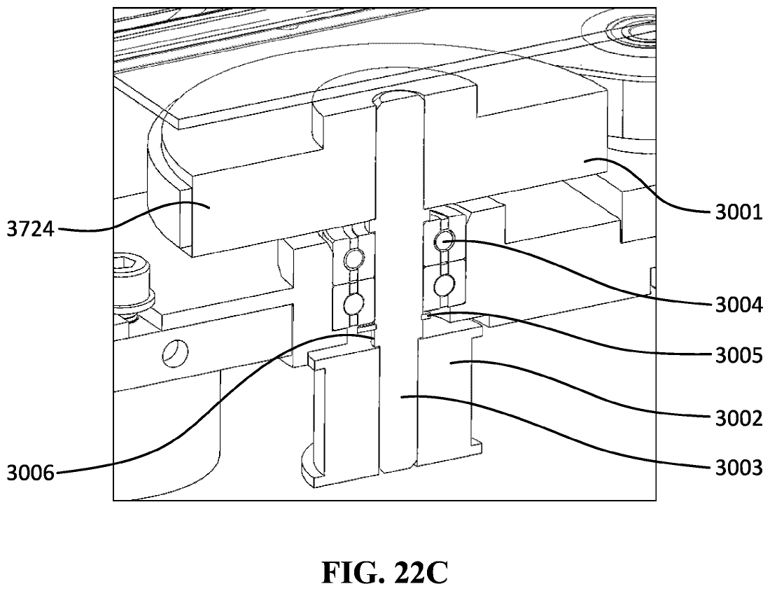

FIGS. 22A-22C illustrate another implementation of a theta-axis robot that may be compared to theta-axis robots 720 and 1720. As shown, theta-axis robot 3720 includes motor 3721, belts 3722 and 3727, idler pulleys 3723 and 3724, platform 3725, coupling 3726, and cover 3728. Idler pulleys 3723 and 3724 are connected via belt 3722. Idler pulley 3724 is also connected to a drive pully (not shown) positioned beneath motor 3721 via belt 3727. The drive pulley is directly coupled to a shaft of motor 3721. When motor 3721 causes the drive pulley to rotate, idler pulleys 3723 and 3724 will also rotate. Theta-axis robot 3720 may be slidingly coupled to a rail (e.g., rail 711) via coupling 3726.

As shown, theta-axis robot 3720 also includes two belt tensioners. One belt tensioner includes idler pulley 3791, plate 3792, recess 3793, screw 3794, and opening 3795. Idler pulley 3791 contacts belt 3722 and may be used to apply tension to belt 3722 and to prevent it from slipping. Idler pulley 3791 is rotatably coupled to plate 3792. Plate 3792 is positioned within a recess 3793 of platform 3725. Plate 3792 is coupled to screw 3794, which extends through opening 3795 of platform 3725. When screw 3794 is loose, plate 3792 can slide. When screw 3794 is tightened, plate 3792 is prevented from sliding. In some implementations, the first belt tensioner may include one or more springs (not shown) to automatically set the desired tension.

The second belt tensioner includes idler pulley 3796, plate 3797, screw 3799, and opening 3798. Idler pulley 3796 contacts belt 3727 and may be used to apply tension to belt 3727 and to prevent it from slipping. Idler pulley 3796 is rotatably coupled to plate 3797. Plate 3797 is positioned within a recess (not shown) of platform 3725. Plate 3797 is coupled to screw 3799, which extends through opening 3798 of platform 3725. When screw 3799 is loose, plate 3797 can slide. When screw 3799 is tightened, plate 3797 is prevented from sliding. In some implementations, the second belt tensioner may include one or more springs (not shown) to automatically set the desired tension. In some implementations, motor 3721 may be mounted to a sub-plate that can pivot. The pivoting motion can provide the tension without, for example, the use of plate 3797 and idler pulley 3796.

As shown in FIG. 22C, idler pulley 3724 includes top disc 3001, bottom disc 3002, shaft 3003, ball bearings 3004, wave ring 3005, and retaining rings 3006. Top disc 3001 contacts belt 3727 and can be seen from the perspective of FIG. 22A. Bottom disc 3002 contacts belt 3722 and can be seen from the perspective of FIG. 22B. Top disc 3001 and bottom disc 3002 are fixedly coupled to each other via shaft 3003. Shaft 3003 is configured to rotate with ball bearings 3004. In comparison to theta-axis robot 720, theta-axis robot 3720 advantageously has an increased torque ratio. This is accomplished by sizing top disc 3001 and bottom disc 3002 such that the diameter of top disc 3001 is greater than the diameter of bottom disc 3002. The increased gear ratio may, for example, improve the control resolution of robotic subsystem 700. Thus, in some implementations, theta-axis robot 720 may be replaced with theta-axis robot 3720. In some implementations, ball bearings 3004 may be repositioned such that, for example, one is positioned above top disc 3001 and another is positioned below top disc 3001 (from the perspective of FIG. 22C). Such an arrangement may advantageously provide additional rigidity to shaft 3003.

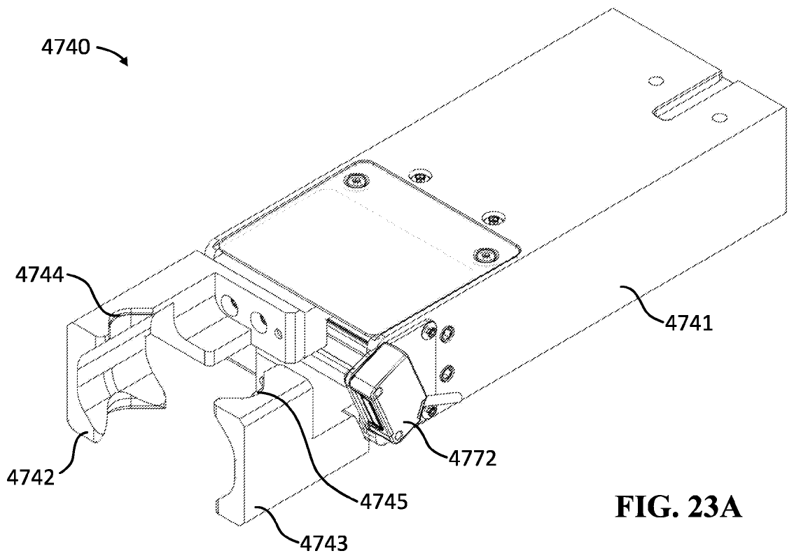

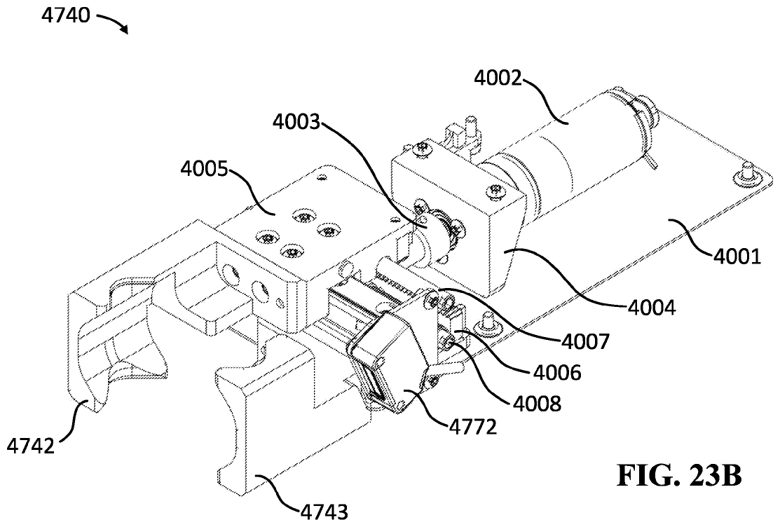

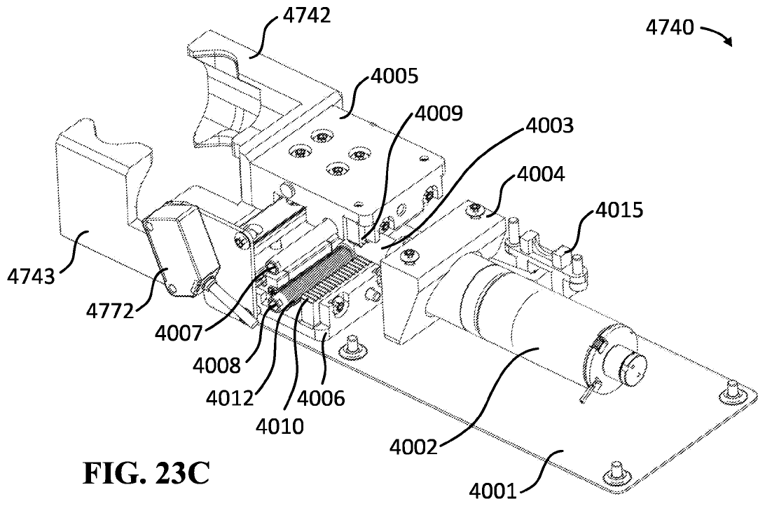

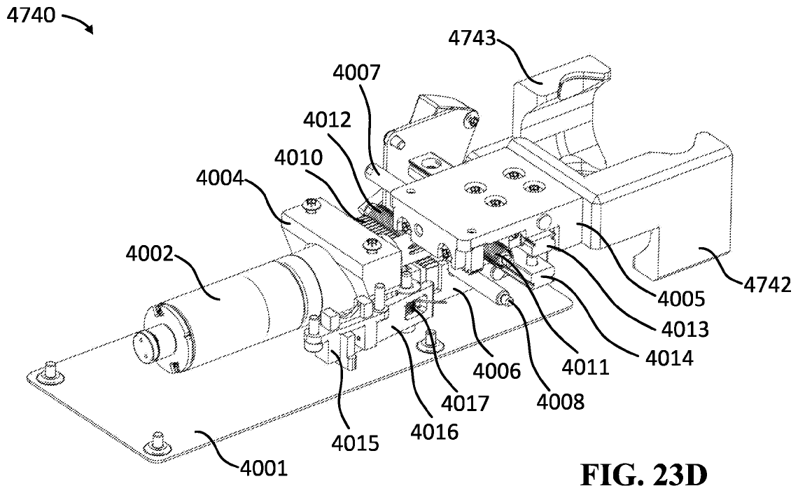

FIG. 23A illustrates another implementation of a gripper assembly that may be compared to gripper assemblies 740, 1740, and 2740. As shown, gripper assembly 4740 includes housing 4741, grippers 4742 and 4743, recesses 4744 and 4745, and sensor 4772. During operation, a motor within housing 4741 may be used to move grippers 4742 and 4743 closer together or farther apart. These motions may enable a robotic subsystem (e.g., robotic subsystem 700) to grab and release sample containers. Furthermore, the sample containers may be oriented vertically or horizontally. When a sample container is in an upright position, recesses 4744 and 4745 may be used to grasp a neck of the sample container. When a sample container is in a horizontal position, the curved portion of the body of grippers 4742 and 4743 may be used to grasp a bottom end of the sample container.

In some implementations, sensor 4772 may be a non-contact sensor, such as an optical sensor or an ultrasonic sensor. In some implementations, sensor 4772 may be used to verify movements of a robotic subsystem (e.g., robotic subsystem 700). For example, sensor 4772 may be used to verify that gripper assembly 4740 has successfully grabbed or released a sample container. As another example, sensor 4772 may be used to verify that gripper assembly 4740 is positioned correctly relative to one or more components of a module (e.g., output chutes 320, compartments 330 and 340, imaging subsystem 500, or waste management subsystem 600). By tilting sensor 4772 (as opposed to orienting it vertically or horizontally), it can advantageously be used to measure a vertical and/or horizontal distance between gripper assembly 4740 and another object. Furthermore, by positioning sensor 4772 such that it does not extend above or below housing 4741, sensor 4772 advantageously does not interfere with the movements of gripper assembly 4740. However, in some implementations, the orientation and/or position of sensor 4772 may be changed. For example, in some implementations, sensor 4772 may be oriented vertically or horizontally. As another example, in some implementations, sensor 4772 may be coupled to one of grippers 4742 and 4743 instead of housing 4741. In some implementations, sensor 4772 may communicate with one or more controllers (e.g., controllers 751, 752, and/or 754) and/or computers (e.g., computer 231 and/or 370) through one or more electrical conduits or wireless communications. In some implementations, gripper assembly 4740 may include one or more additional sensors that can also be used to verify movements of a robotic subsystem. In some implementations, sensor 4772 may be removed from gripper assembly 4740.

FIGS. 23B-23D are perspective views of gripper assembly 4740 without portions of housing 4741. As shown, gripper assembly 4740 includes platform 4001, motor 4002, pinion gear 4003, mount 4004, blocks 4005 and 4006, shafts 4007 and 4008, gear racks 4009 and 4010, springs 4011, 4012, and 4017, rails 4013 and 4014, sensor 4015, and member 4016. Block 4005 is coupled to gripper 4742 and gear rack 4009. Similarly, block 4006 is coupled to gripper 4743 and gear rack 4010. Block 4005 is slidingly coupled to shaft 4007 and rail 4013. Similarly, block 4006 is slidingly coupled to shaft 4008 and rail 4014. Motor 4002 is configured to rotate pinion gear 4003. In some implementations, motor 4002 is a compact brushless DC motor with a gear reducer and/or encoder. Pinion gear 4003 is engaged with gear racks 4009 and 4010 such that as pinion gear 4003 rotates in a first direction, grippers 4742 and 4743 are moved closer together. Similarly, as pinion gear 4003 rotates in the opposite direction, grippers 4742 and 4743 are moved farther apart. Springs 4011 and 4012 are positioned around shafts 4007 and 4008, respectively, and configured to apply a force to blocks 4005 and 4006, respectively, that causes grippers 4742 and 4743 to move closer together. Thus, in the event of a power failure, springs 4011 and 4012 may prevent a robotic subsystem (e.g., robotic subsystem 700) from dropping a sample container.

Sensor 4015, member 4016, and spring 4017 may be used to measure the position of grippers 4742 and 4743. For example, spring 4017 may apply a force to member 4016 in a first direction. However, as grippers 4742 and 4743 move closer together, block 4006 may cause member 4016 to rotate in the opposite direction. This rotation causes a portion of member 4016 to move out from underneath sensor 4015. This movement may then be detected by sensor 4015. In some implementations, sensor 4015 may communicate with one or more controllers (e.g., controllers 751, 752, and/or 754) and/or computers (e.g., computer 231 and/or 370) through one or more electrical conduits or wireless communications. In some implementations, sensor 4015 may be a non-contact sensor, such as an optical sensor. In other implementations, sensor 4015 may, for example, be a touch sensor.

In comparison to gripper assembly 740, gripper assembly 4740 may advantageously have an increased stroke length and/or a reduced width. This is accomplished by using two independent linear guide assemblies. Most grippers use a single linear guide with two carriages mounted to the same rail. This restricts the stroke because the carriages share the same guide rail. By using two linear guides, the carriages can pass each other and therefore provide more stroke in a reduced width footprint. Thus, in some implementations, gripper assembly 740 may be replaced with gripper assembly 4740.

In some implementations, motor 4002 may be a servo motor. In such implementations, motor 4002 may advantageously enable gripper assembly 4740 to apply an additionally squeezing force via grippers 4742 and 4743 to compensate for misalignment during pickups. Additionally, in such implementations, motor 4002 may advantageously provide both torque and position measurements. In some implementations, one or more controllers (e.g., controllers 751, 752, and/or 754) and/or computers (e.g., computer 231 and/or 370) may use these torque and position measurements to detect whether a sample container is positioned within grippers 4742 and 4743. Similarly, in some implementations, one or more controllers and/or computers may use these torque and position measurements to detect whether a sample container has been dropped. In some implementations, motor 4002 may be another type of motor, such as a stepper motor.

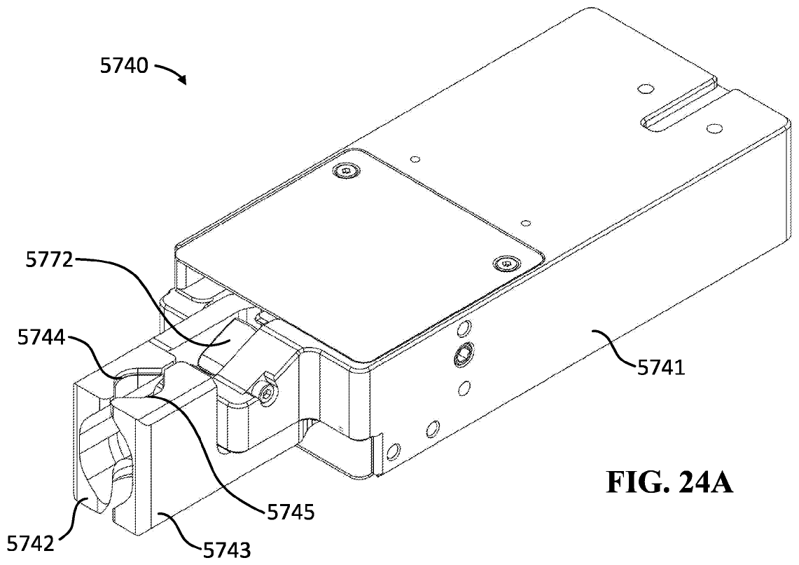

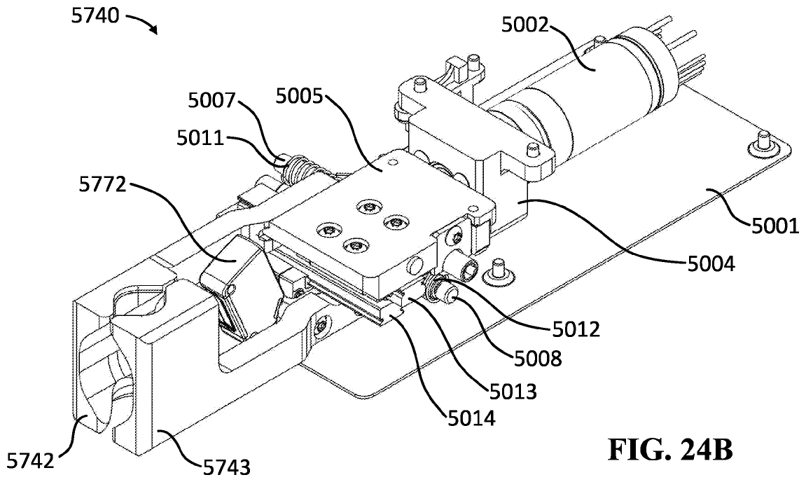

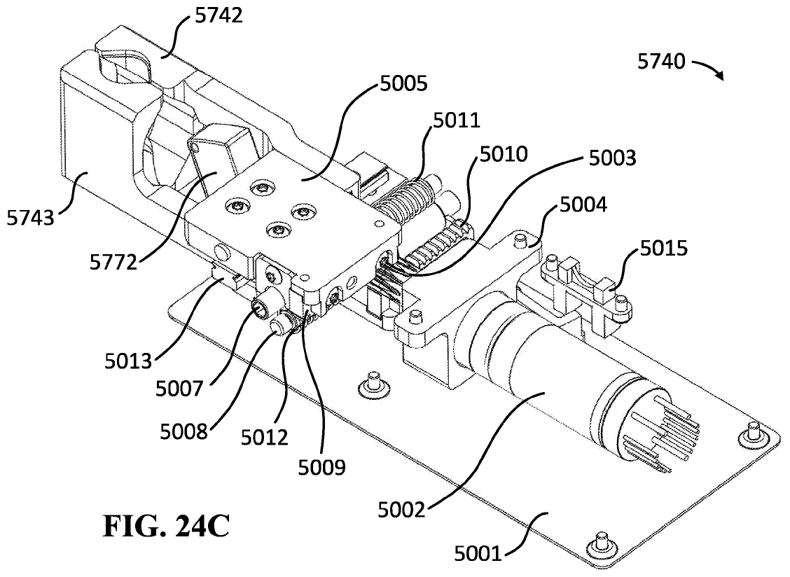

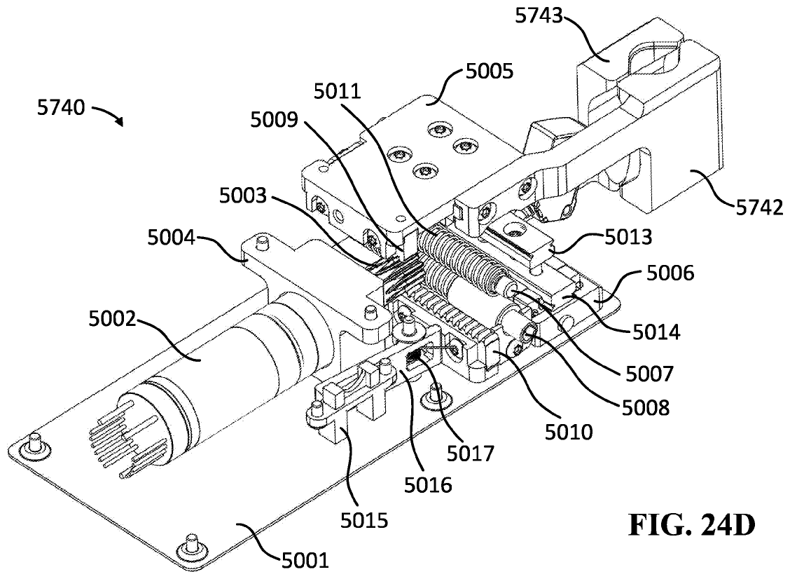

FIG. 24A illustrates another implementation of a gripper assembly that may be compared to gripper assemblies 740, 1740, 2740, and 4740. As shown, gripper assembly 5740 includes housing 5741, grippers 5742 and 5743, recesses 5744 and 5745, and sensor 5772. During operation, a motor within housing 5741 may be used to move grippers 5742 and 5743 closer together or farther apart. These motions may enable a robotic subsystem (e.g., robotic subsystem 700) to grab and release sample containers. Furthermore, the sample containers may be oriented vertically or horizontally. When a sample container is in an upright position, recesses 5744 and 5745 may be used to grasp a neck of the sample container. When a sample container is in a horizontal position, the curved portion of the body of grippers 5742 and 5743 may be used to grasp a bottom end of the sample container.

In some implementations, sensor 5772 may be a non-contact sensor, such as an optical sensor or an ultrasonic sensor. In some implementations, sensor 5772 may be used to verify movements of a robotic subsystem (e.g., robotic subsystem 700). For example, sensor 5772 may be used to verify that gripper assembly 5740 has successfully grabbed or released a sample container. As another example, sensor 5772 may be used to verify that gripper assembly 5740 is positioned correctly relative to one or more components of a module (e.g., output chutes 320, compartments 330 and 340, imaging subsystem 500, or waste management subsystem 600). By tilting sensor 5772 (as opposed to orienting it vertically or horizontally), it can advantageously be used to measure a vertical and/or horizontal distance between gripper assembly 5740 and another object. Furthermore, by positioning sensor 5772 between grippers 5742 and 5743, it can accurately verify movements of a robotic subsystem. Additionally, by positioning sensor 5772 such that it does not extend above or below grippers 5742 and 5743, sensor 5772 advantageously does not interfere with the movements of gripper assembly 5740. However, in some implementations, the orientation and/or position of sensor 5772 may be changed. For example, in some implementations, sensor 5772 may be oriented vertically or horizontally. As another example, in some implementations, sensor 5772 may be coupled to one of grippers 5742 and 5743 instead of housing 5741. In some implementations, sensor 5772 may communicate with one or more controllers (e.g., controllers 751, 752, and/or 754) and/or computers (e.g., computer 231 and/or 370) through one or more electrical conduits or wireless communications. In some implementations, gripper assembly 5740 may include one or more additional sensors that can also be used to verify movements of a robotic subsystem. In some implementations, sensor 5772 may be removed from gripper assembly 5740.

FIGS. 24B-24D are perspective views of gripper assembly 5740 without portions of housing 5741. As shown, gripper assembly 5740 includes platform 5001, motor 5002, pinion gear 5003, mount 5004, blocks 5005 and 5006, shafts 5007 and 5008, gear racks 5009 and 5010, springs 5011, 5012, and 5017, rails 5013 and 5014, sensor 5015, and member 5016. Block 5005 is coupled to gripper 5742 and gear rack 5009. Similarly, block 5006 is coupled to gripper 5743 and gear rack 5010. Block 5005 is slidingly coupled to shaft 5007 and rail 5013. Similarly, block 5006 is slidingly coupled to shaft 5008 and rail 5014. Motor 5002 is configured to rotate pinion gear 5003. In some implementations, motor 5002 is a compact brushless DC motor with a gear reducer and/or encoder. Pinion gear 5003 is engaged with gear racks 5009 and 5010 such that as pinion gear 5003 rotates in a first direction, grippers 5742 and 5743 are moved closer together. Similarly, as pinion gear 5003 rotates in the opposite direction, grippers 5742 and 5743 are moved farther apart. Springs 5011 and 5012 are positioned around shafts 5007 and 5008, respectively, and configured to apply a force to blocks 5005 and 5006, respectively, that causes grippers 5742 and 5743 to move closer together. Thus, in the event of a power failure, springs 5011 and 5012 may prevent a robotic subsystem (e.g., robotic subsystem 700) from dropping a sample container.

Sensor 5015, member 5016, and spring 5017 may be used to measure the position of grippers 5742 and 5743. For example, spring 5017 may apply a force to member 5016 in a first direction. However, as grippers 5742 and 5743 move closer together, block 5006 may cause member 5016 to rotate in the opposite direction. This rotation causes a portion of member 5016 to move out from underneath sensor 5015. This movement may then be detected by sensor 5015. In some implementations, sensor 5015 may communicate with one or more controllers (e.g., controllers 751, 752, and/or 754) and/or computers (e.g., computer 231 and/or 370) through one or more electrical conduits or wireless communications. In some implementations, sensor 5015 may be a non-contact sensor, such as an optical sensor. In other implementations, sensor 5015 may, for example, be a touch sensor.

In comparison to gripper assembly 740, gripper assembly 5740 may advantageously have an increased stroke length and/or a reduced width. This is accomplished by using two independent linear guide assemblies. Most grippers use a single linear guide with two carriages mounted to the same rail. This restricts the stroke because the carriages share the same guide rail. By using two linear guides, the carriages can pass each other and therefore provide more stroke in a reduced width footprint. Thus, in some implementations, gripper assembly 740 may be replaced with gripper assembly 5740.

In some implementations, motor 5002 may be a servo motor. In such implementations, motor 5002 may advantageously enable gripper assembly 5740 to apply an additionally squeezing force via grippers 5742 and 5743 to compensate for misalignment during pickups. Additionally, in such implementations, motor 5002 may advantageously provide both torque and position measurements. In some implementations, one or more controllers (e.g., controllers 751, 752, and/or 754) and/or computers (e.g., computer 231 and/or 370) may use these torque and position measurements to detect whether a sample container is positioned within grippers 5742 and 5743. Similarly, in some implementations, one or more controllers and/or computers may use these torque and position measurements to detect whether a sample container has been dropped. In some implementations, motor 5002 may be another type of motor, such as a stepper motor.

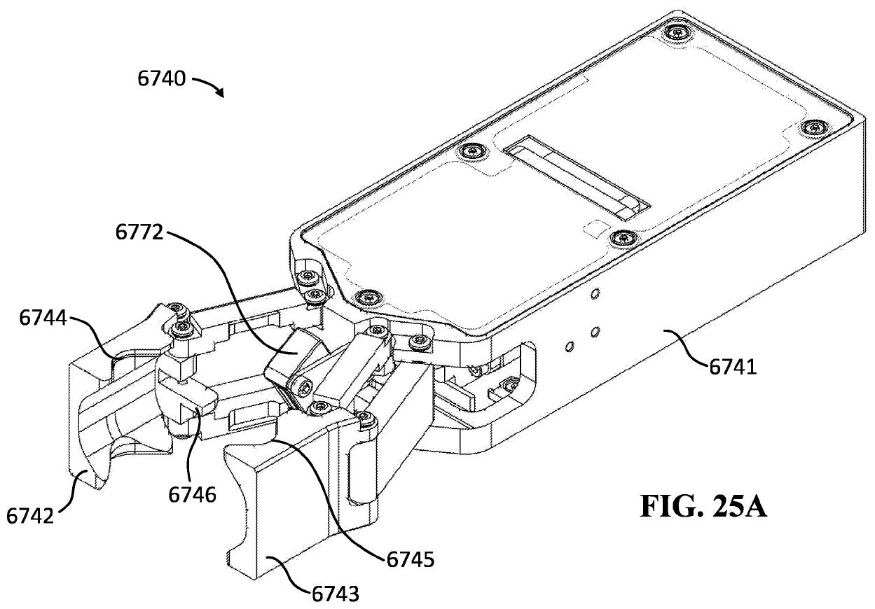

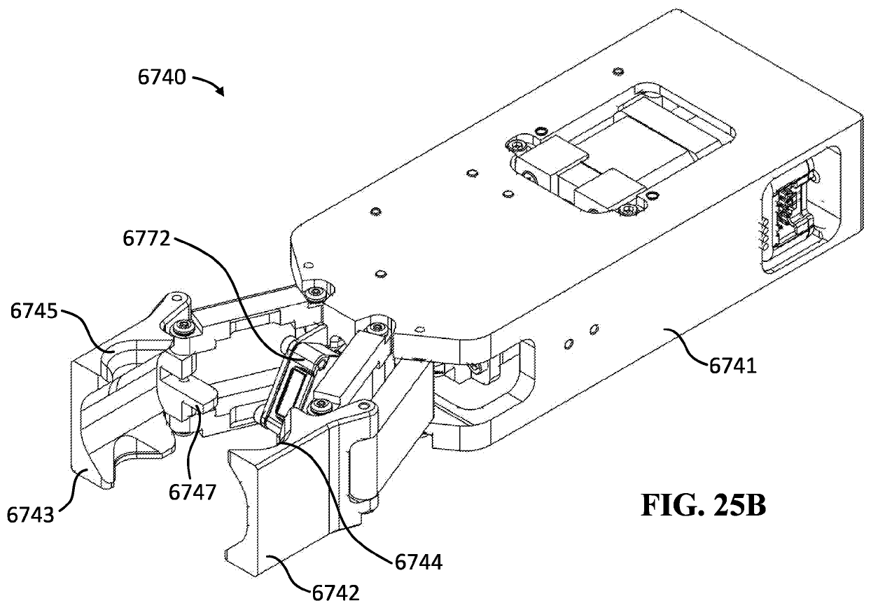

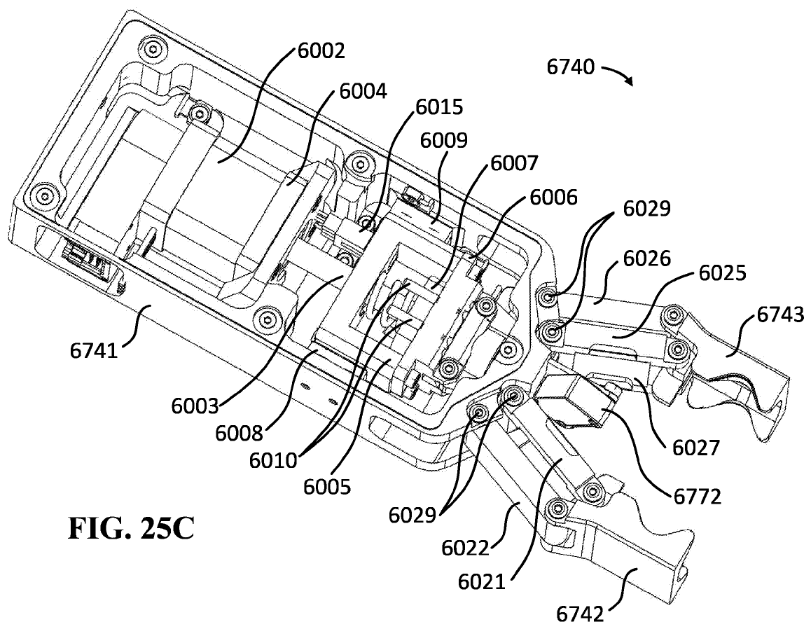

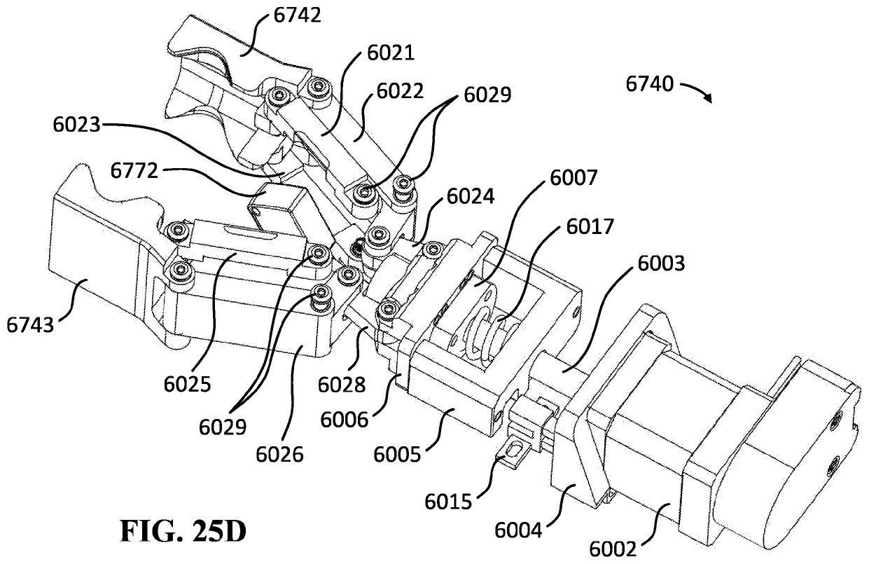

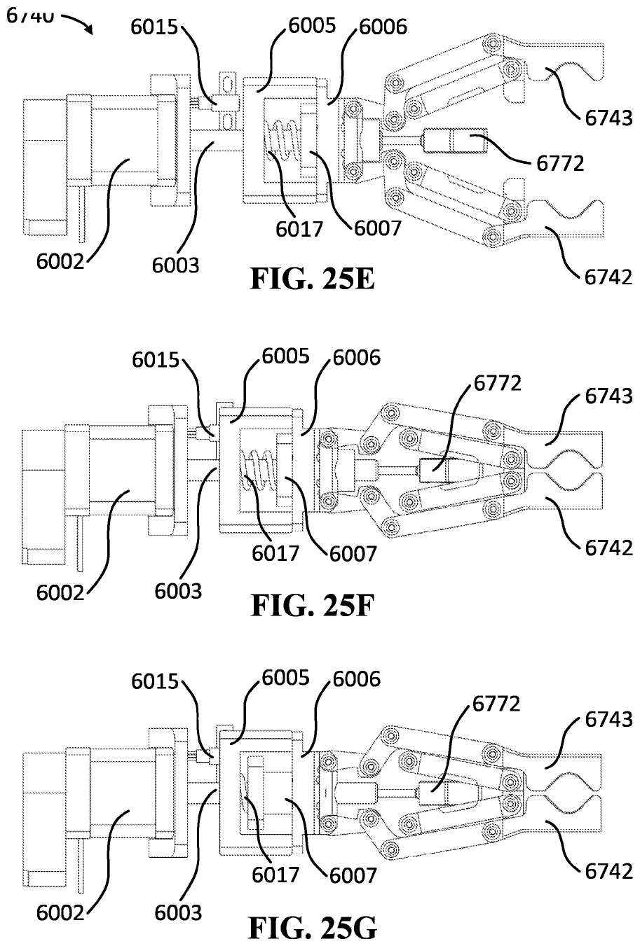

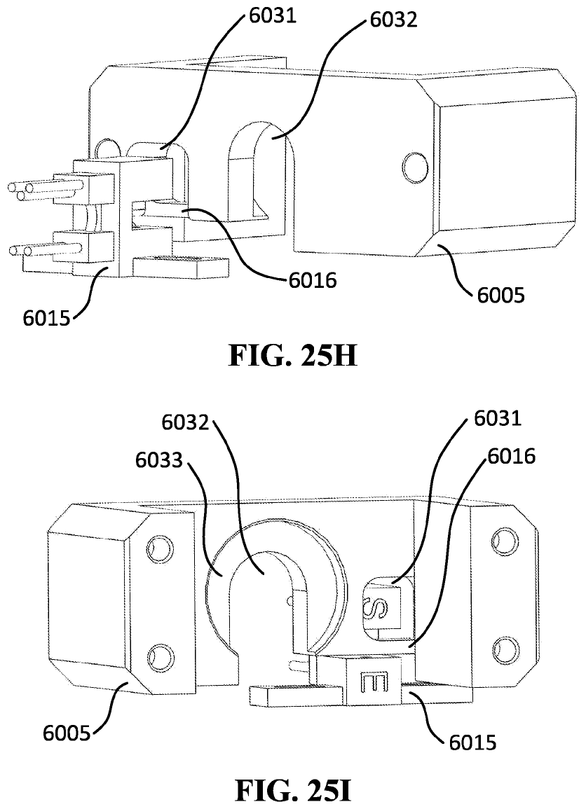

FIGS. 25A and 25B illustrate top and bottom perspective views, respectively, of another implementation of a gripper assembly that may be compared to gripper assemblies 740, 1740, 2740, 4740, and 5740. As shown, gripper assembly 6740 includes housing 6741, grippers 6742 and 6743, recesses 6744 and 6745, engagement features 6746 and 6747, and sensor 6772. During operation, a motor within housing 6741 may be used to move grippers 6742 and 6743 closer together or farther apart. These motions may enable a robotic subsystem (e.g., robotic subsystem 700) to grab and release sample containers. Furthermore, the sample containers may be oriented vertically or horizontally. When a sample container is in an upright position, recesses 6744 and 6745 may be used to grasp a neck of the sample container. Additionally, when grippers 6742 and 6743 grasp the neck of the sample container, engagement features 6746 and 6747 may interlock with one another to provide additional support. When a sample container is in a horizontal position, the curved portion of the body of grippers 6742 and 6743 may be used to grasp a bottom end of the sample container.

In some implementations, sensor 6772 may be a non-contact sensor, such as an optical sensor or an ultrasonic sensor. In some implementations, sensor 6772 may be used to verify movements of a robotic subsystem (e.g., robotic subsystem 700). For example, sensor 6772 may be used to verify that gripper assembly 6740 has successfully grabbed or released a sample container. As another example, sensor 6772 may be used to verify that gripper assembly 6740 is positioned correctly relative to one or more components of a module (e.g., output chutes 320, compartments 330 and 340, imaging subsystem 500, or waste management subsystem 600). By tilting sensor 6772 (as opposed to orienting it vertically or horizontally), it can advantageously be used to measure a vertical and/or horizontal distance between gripper assembly 6740 and another object. Furthermore, by positioning sensor 6772 between grippers 6742 and 6743, it can accurately verify movements of a robotic subsystem. Additionally, by positioning sensor 6772 such that it does not extend above or below grippers 6742 and 6743, sensor 6772 advantageously does not interfere with the movements of gripper assembly 6740. However, in some implementations, the orientation and/or position of sensor 6772 may be changed. For example, in some implementations, sensor 6772 may be oriented vertically or horizontally. As another example, in some implementations, sensor 6772 may be coupled to one of grippers 6742 and 6743 instead of housing 6741. In some implementations, sensor 6772 may communicate with one or more controllers (e.g., controllers 751, 752, and/or 754) and/or computers (e.g., computer 231 and/or 370) through one or more electrical conduits or wireless communications. In some implementations, gripper assembly 6740 may include one or more additional sensors that can also be used to verify movements of a robotic subsystem. In some implementations, sensor 6772 may be removed from gripper assembly 6740.

FIG. 25C is a perspective view of gripper assembly 6740 without a top portion of housing 6741. FIG. 25D is a perspective view of gripper assembly 6740 without housing 6741 and some other stationary components. As shown, gripper assembly 6740 includes motor 6002, shaft 6003, mount 6004, spring plate 6005, nut base 6006, flanged screw nut 6007, pads 6008 and 6009, pins 6010, sensor 6015, spring 6017, members 6021-6028, and couplings 6029. Shaft 6003 is threaded. Spring plate 6005 is slidingly engaged with shaft 6003. Pads 6008 and 6009 are coupled to housing 6741 and configured to help prevent spring plate 6005 from rotating as it slides along shaft 6003. Flanged screw nut 6007 is engaged with the threads of shaft 6003. In some implementations, flanged screw nut 6007 is a flanged ball screw nut. Nut base 6006 is coupled to spring plate 6005 and slidingly engaged with flanged screw nut 6007. Pins 6010 are coupled to nut base 6006 and configured to help prevent flanged screw nut 6007 from rotating as it moves along shaft 6003. In this particular implementation, two pins are positioned above nut base 6006 and two pins (not shown) are positioned below nut base 6006. In other implementations, more or less pins may be included to prevent flanged screw nut 6007 from rotating as it moves along shaft 6003. Spring 6017 is slidingly engaged with shaft 6003. A first end of spring 6017 contacts spring plate 6005. A second end of spring 6017, opposite the first end, contacts flanged screw nut 6007.

As shaft 6003 rotates in a first direction, flanged screw nut 6007 moves toward motor 6002. As flanged screw nut 6007 moves toward motor 6002, it pushes spring 6017 towards motor 6002. If there is minimal or no resistance between grippers 6742 and 6743, the pushing force generated by flanged screw nut 6007 is translated to spring plate 6005 (and consequently nut base 6006) via spring 6017. As a result, spring plate 6005, nut base 6006, and spring 6017 will also move towards motor 6002 as flanged screw nut 6007 moves toward motor 6002. If there is resistance between grippers 6742 and 6743 (e.g., when grippers 6742 and 6743 contact one another or when a sample container is positioned within grippers 6742 and 6743), the pushing force generated by flanged screw nut 6007 will cause spring 6017 to compress. As a result, spring plate 6005 and nut base 6006 may remain relatively stationary as flanged screw nut 6007 moves toward motor 6002. As shaft 6003 rotates in a second direction, opposite the first direction, flanged screw nut 6007 moves away from motor 6002. As flanged screw nut 6007 moves away from motor 6002, it pushes nut base 6006 (and consequently spring plate 6005 and spring 6017) away from motor 6002.

Grippers 6742 and 6743 are coupled to nut base 6006 via members 6021-6028 and couplings 6029. As shown, gripper 6742 is rotatably coupled to a first end of members 6021-6023 via couplings 6029. Similarly, gripper 6743 is rotatably coupled to a first end of members 6025-6027 via couplings 6029. A second end of members 6021, 6023, 6025, and 6027, opposite the first end, is rotatably coupled to housing 6741 via couplings 6029. A second end of members 6022 and 6026, opposite the first end, is rotatably coupled to a first end of members 6024 and 6028, respectively, via couplings 6029. A second end of members 6024 and 6028, opposite the first end, is rotatably coupled to nut base 6006 via couplings 6029. As shown, members 6021, 6023, 6025, and 6027 are straight, and members 6022, 6024, 6026, and 6028 are bent. Furthermore, the corners of members 6022 and 6026 are rotatably coupled to housing 6741 via couplings 6029.

FIGS. 25E-25G are top-down views of gripper assembly 6740 without housing 6741 and some other stationary components (see also FIG. 25D) that illustrate how grippers 6742 and 6743 are moved. As best seen in FIGS. 25E and 25F, as nut base 6006 moves toward motor 6002, it pulls members 6024 and 6028 toward motor 6002. The pulling force on members 6024 and 6028 also causes them to rotate about axes that extend through the couplings 6029 at the second ends of members 6024 and 6028. The rotation of members 6024 and 6028 also causes (a) members 6022 and 6026, respectively, to rotate about axes that extend through the couplings 6029 at the corners of members 6022 and 6026 and (b) members 6021, 6023, 6025, and 6027 to rotate about axes that extend through the couplings 6029 at the second ends of members 6021, 6023, 6025, and 6027. Collectively, the rotation of members 6021-6023 and 6025-6027 causes grippers 6742 and 6743 to move closer together. As nut base 6006 moves away from motor 6002, it pushes members 6024 and 6028 away from motor 6002. In much the same way that the pulling force on members 6024 and 6028 also caused members 6021-6028 to rotate, the pushing force on members 6024 and 6028 also causes members 6021-6028 to rotate, but in the opposite direction. As a result, grippers 6742 and 6743 move farther apart. As best seen in FIG. 25G, as nut base 6006 moves toward motor 6002, it may merely compress spring 6017 rather than move grippers 6742 and 6743 closer together if there is resistance between grippers 6742 and 6743. In FIG. 25G, this resistance is caused by grippers 6742 and 6743 contacting one another. However, a similar resistance may also be generated when an object, such as a sample container, is positioned between grippers 6742 and 6743.

FIGS. 25H and 25I are perspective views of spring plate 6005 and sensor 6015. As shown, spring plate 6005 includes member 6016, openings 6031 and 6032, and recess 6033. Shaft 6003 extends through opening 6032. The first end of spring 6017 is positioned within recess 6033 of spring plate 6005. Spring plate 6005 and sensor 6015 may be used to measure the position of grippers 5742 and 5743. For example, as spring plate 6005 moves toward motor 6002 and away from motor 6002, the corresponding movements of member 6016 may be detected by sensor 6015. For example, when spring plate 6005 is in the position illustrated in FIGS. 25E and 25G, member 6016 is not positioned underneath sensor 6015. Furthermore, when spring plate 6005 is in the position illustrated in FIG. 25F, member 6016 is positioned underneath sensor 6015. Additionally, when spring plate 6005 is in the position illustrated in FIG. 25F, a portion of sensor 6015 may extend through opening 6031. In some implementations, sensor 6015 may communicate with one or more controllers (e.g., controllers 751, 752, and/or 754) and/or computers (e.g., computer 231 and/or 370) through one or more electrical conduits or wireless communications. In some implementations, sensor 6015 may be a non-contact sensor, such as an optical sensor. In other implementations, sensor 6015 may, for example, be a touch sensor. In some implementations, the shape of spring plate 6005 may be changed. For example, opening 6031 may be filled in. In some implementations, the orientation and/or position of sensor 6015 may be changed. For example, in some implementations, sensor 6015 may be oriented vertically instead of horizontally. As another example, in some implementations, sensor 6015 may be repositioned to detect the movements of another component of gripper assembly 6740, such as nut base 6006 or flanged screw nut 6007.

In some implementations, motor 6002 may be a stepper motor. In comparison to, for example, a servo motor, a stepper motor may have a lower cost. However, a stepper motor may not provide the same torque and/or feedback. In the implementation illustrated in FIGS. 25A-25I, spring 6017 can advantageously compensate for the reduced torque of a stepper motor and/or misalignment during pickups. Spring 6017 can also advantageously simplify the programming of gripper assembly 6740. For example, one or more controllers (e.g., controllers 751, 752, and/or 754) and/or computers (e.g., computer 231 and/or 370) may drive motor 6002 to a predetermined position and spring 6017 may compensate for any errors at that position (e.g., misalignment). In some implementations, motor 6002 may be another type of motor, such as a servo motor.

Various modifications can be made to gripper assembly 6740. For example, in some implementations, one or more components may be modified and/or removed. For example, in some implementations, spring plate 6005, flanged screw nut 6007, and/or spring 6017 may be removed. In some such implementations, nut base 6006 may be modified to be engaged with the threads of shaft 6003. As another example, one or more of members 6021-6028 may be modified and/or removed. For example, in some implementations, members 6021 and 6023 can be combined into a single member. Similarly, in some implementations, members 6025 and 6027 can be combined into a single member. Such implementations may advantageously include less moving parts. However, such implementations may also include less space for sensor 6772. In the implementation illustrated in FIGS. 25A-25I, members 6021, 6023, 6025, and 6027 are advantageously positioned either above or below sensor 6772. Additionally, members 6021, 6023, 6025, and 6027 include a recess to ensure that they do not contact sensor 6772.

### Weight Measurement Subsystem

As explained above, when processing blood culture bottles in a laboratory environment that is processing a large number of blood culture bottles, there is a need to be able to monitor the fill condition of each bottle accurately. The amount of sample collected can, for example, directly impact the likelihood of detecting a bacterial and/or fungal infection. Therefore, in some implementations, in addition to using imaging subsystem 500 to determine the presence or absence of foam and/or the fill level, automated system 100 may include a scale to verify any determinations made with imaging subsystem 500. Furthermore, in some implementations, automated system 100 may use a scale instead of imaging subsystem 500 to monitor the fill condition of each sample container. In some implementations, the scale may, for example, be incorporated into modules 101 or 102. The scale may also be an external device in communication with modules 101 and 102.

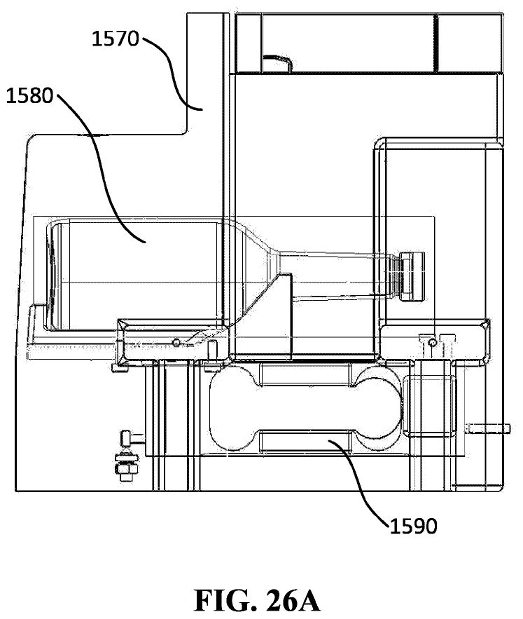

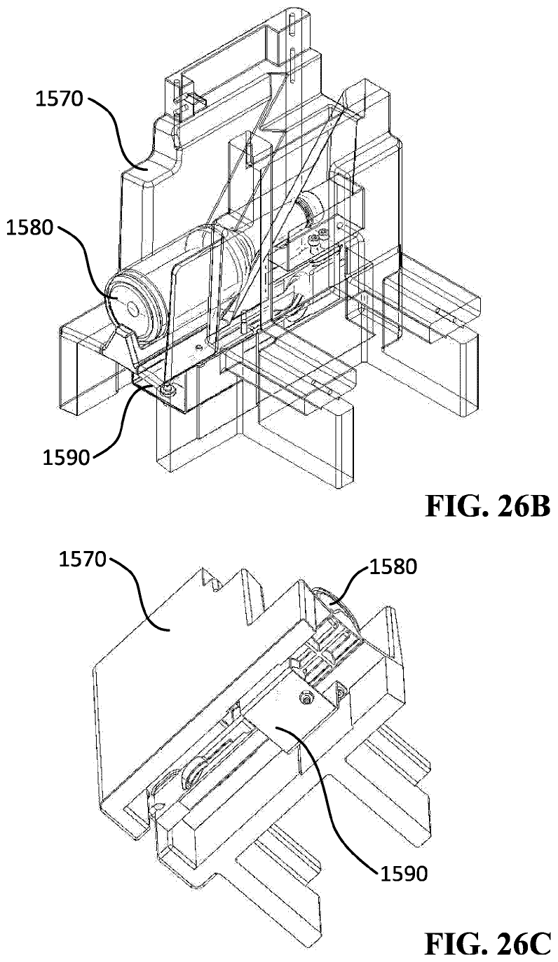

FIGS. 26A-26C illustrate an implementation in which a load cell is incorporated into a chute that may be positioned within module 101. As shown, load cell 1590 is positioned beneath chute 1570 such that it can measure the weight of sample container 1580 (e.g., a blood culture bottle). Chute 1570 may be compared to chute 570 of imaging subsystem 500. In some implementations, the assembly illustrated in FIGS. 26A-26C (e.g., load cell 1590 and chute 1570) may replace chute 570. In such implementations, a sample container may be weighed immediately after being imaged by camera 510.

£ By itself, the measured weight of a particular sample container may not readily indicate whether the sample container is overfilled or underfilled. The unfilled tare weight of every individual sample container may not be available. Furthermore, using one standard tare weight may not provide insufficient accuracy due to the large stack of production tolerances affecting weight. Next to variations in production of the sample container itself, the fill levels of sensor material, culture media, and media beads have a big influence on the tare weight of a sample container before a sample is even added.

To address these manufacturing inconsistencies, a correction factor may be applied to an average unfilled tare weight. For example, a camera (e.g., camera 510) may capture images of sample container 1580. The distance between a fill line (not shown) on sample container 1580 and a reference surface of sample container 1580 (e.g., the bottom of sample container 1580) may be measured from the images captured by the camera (e.g., using computer 231 and/or 370). In contrast to, for example, the top surface of a liquid in sample container 1580, which can be obfuscated by bubbles and media beads clinging to the inside of sample container 1580 or stuck in a neck of sample container 1580, the fill line can be reliably and accurately detected through imaging. The distance between the fill line and the reference surface may be compared to a predetermined distance to calculate a correction factor. The predetermined distance may, for example, correspond to a standard distance between a fill line and a bottom of a sample container. The corrected unfilled tare weight may then be compared to the weight measured by scale 1590 to determine whether sample container 1580 is overfilled or underfilled.

While the correction factor described above may improve the overall accuracy of the system, it may not always be possible to calculate. For example, sample containers may not always have a fill line. Therefore, in some implementations, a measured weight may be directly compared to an average unfilled tare weight (as opposed to a corrected unfilled tare weight) to determine whether a sample container is overfilled or underfilled. In such implementations, a larger margin of error may be reported to a user (e.g., via display 310). In some implementations, the average unfilled tare weight may be selected based on the contents of a sample container (e.g., media type) to further improve accuracy. In some implementations, the average unfilled tare weight may be selected based on a lot or batch in which the sample container was manufactured to further improve accuracy. For example, while the time required to weigh each sample container during manufacturing may be prohibitively expensive, it may be more cost-effective to weigh one or more representative samples from each lot or batch to calculate an average unfilled tare weight for that particular lot or batch. In some implementations, the average unfilled tare weight may be derived from a label on the sample container or received by module 101 from an external device (e.g., a server) over a network. In some implementations, automated system 100 may be configured to check (e.g., using camera 510) each sample container for any alterations that might affect the accuracy of the techniques described above (e.g., the removal of a cap and/or the addition of more labels by a user). In some implementations, automated system 100 may be configured to compensate for such alterations.

In some implementations, the techniques described above can be used to provide sample volume measurements. By comparing a measured weight to an average unfilled tare weight or a corrected unfilled tare weight, the weight of the sample can be obtained. For example, the average unfilled tare weight can be subtracted from the measured weight to obtain the weight of the sample. The weight of the sample can then be converted into a volume using a predetermined density value. For example, if the sample container is a blood culture bottle, a predetermined density value for blood can be used to convert the weight of the blood sample into a volume measurement. In some implementations, the calculated sample volume measurement can be displayed (e.g., via display 310).

In some implementations, sample containers may be individually weighed with a scale and scanned before being placed in an instrument, such as module 101. In some implementations, the scale may include an indicator light or something similar to let a user know whether the scale has fully settled. In some implementations, if the measured weight is outside an expected range, a user may be prompted to place the sample container back on the scale and to wait for the scale to indicate it has a stable measurement. In some implementations, if the user ignores the prompt and enters the bottle into module 101, the weight for the sample container may be recorded as zero, indicating no weight for the sample container was obtained.

Individually weighing each sample container could dramatically slow the workflow of entering the sample containers into module 101 by also adding steps of placing each individual sample container on the scale, waiting for the scale to settle, and removing each individual sample container from the scale to complete the loading process by, for example, scanning a barcode on each individual sample container and locating a position for each individual sample container in module 101. To alleviate this problem, a batch of sample containers could be measured at the same time. For example, a batch of sample containers (e.g., a rack of sample containers) may be placed on a scale. After the scale settles, each sample container can be removed one at a time, scanned, and placed in module 101. While the scanning and placing are happening for a particular sample container, the scale will settle and the change in weight can be used to determine the weight of that particular sample container. In some implementations, the scale may include an indicator light or something similar to let a user know whether the scale has fully settled.

In some implementations, the scale used to weigh individual sample containers or batches of sample containers may be a separate device that communicates modules 101 and 102. For example, the scale could be built into the racks used to transport sample containers. In some implementations, the scale may be integrated with one or both of modules 101 and 102. For example, the scale may be integrated into a shelf, a drawer, or the housing of module 101 or 102 (e.g., top panel 132). As another example, the scale may be an add-on device that is configured to couple to the housing of module 101 or 102.

In some implementations, the scale may communicate with computer 231 and/or 370 through one or more electrical conduits or wireless communications. In some implementations, system 100 may compare the measured weights obtained with the scale to a standard net weight to determine whether a sample container is overfilled or underfilled. In some implementations, the standard net weight may be selected based on the contents of a sample container (e.g., media type) and/or a lot or batch in which the sample container was manufactured, which may be derived from a label on the sample container, to further improve accuracy. In some implementations, system 100 may use imaging subsystem 500 to calculate a correction factor in the manner described above to further improve accuracy.

In some implementations, the technique of measuring batches of sample containers described above may be used internally to expedite the workflow within modules 101 and 102. For example, instead of placing a scale somewhere internally to individually weigh sample containers (see, e.g., scale 1590), a scale may be integrated into one or both of compartments 330 and 340 to weigh sample containers in batches. In such implementations, robotic subsystem 700 may transfer a particular sample container from one of compartments 330 and 340 to imaging subsystem 500. Once at imaging subsystem, that sample container can be scanned and the corresponding change in weight of the sample containers in one of compartments 330 and 340 can be used to determine the weight of the particular sample container.

Those skilled in the art will appreciate that many of the subsystems and components described above can be readily adapted to other types of automated systems. For example, there are many examples of automated systems in which there is a need to efficiently measure the weights of a plurality of objects. Those skilled in the art will appreciate that the systems and methods described above for measuring batches of sample containers can be readily adapted to measure the weights of other types of objects.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

From the foregoing and with reference to the various figure drawings, those skilled in the art will appreciate that certain modifications may also be made to the present disclosure without departing from the scope of the same. While several implementations of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular implementations. Those skilled in the art will envision other modifications within the scope and spirit of the claims appended hereto.

1. A sample handling module comprising:
a user interface subsystem configured to receive a plurality of untested sample containers and output a plurality of sample containers that tested positive for microbial growth;
an imaging subsystem configured to scan sample containers for label information;
a waste management subsystem configured to receive a plurality of sample containers that tested negative for microbial growth; and
a robotic subsystem configured to:
transfer each of the plurality of untested sample containers from the user interface subsystem to the imaging subsystem for scanning;
transfer each of the plurality of untested sample containers from the imaging subsystem to an incubation module configured to incubate each of the plurality of untested sample containers and measure microbial growth;
transfer each of the plurality of positive sample containers from the incubation module to the imaging subsystem for scanning;
transfer each of the plurality of positive sample containers from the imaging subsystem to the user interface subsystem for output;
transfer each of the plurality of negative sample containers from the incubation module to the imaging subsystem for scanning; and
transfer each of the plurality of negative sample containers from the imaging subsystem to the waste management subsystem for disposal.

2. The sample handling module of claim 1, wherein the user interface subsystem comprises one or more compartments, each of which is configured to receive individual untested sample containers or a rack of untested sample containers.

3. The sample handling module of claim 1, wherein the user interface subsystem comprises one or more compartments, each of which is configured to output individual positive sample containers or a rack of positive sample containers.

4. The sample handling module of claim 1, wherein the user interface subsystem comprises one or more compartments, each of which is configured to (a) receive individual untested sample containers or a rack of untested sample containers and (b) output individual positive sample containers or a rack of positive sample containers.

5. The sample handling module of claim 1, wherein the user interface subsystem comprises one or more compartments, each of which has a liner with one or more sections, wherein each of the one or more sections comprises:
a plurality of receptacles configured to accept sample containers directly; and
a pair of recesses configured to accept a rack of sample containers.

6. The sample handling module of claim 5, wherein the user interface subsystem further comprises one or more illumination lights, each of which is configured to change color based on the type of sample containers positioned within at least one of the compartments.

7. The sample handling module of claim 5, wherein the user interface subsystem further comprises one or more illumination lights, each of which is configured to (a) change to a first color when at least one of the compartments contains one or more untested sample containers and (b) change to a second color when the at least one compartment contains one or more positive sample containers.

8. The sample handling module of claim 7, wherein the one or more illumination lights are positioned above the liner of the at least one compartment.

9. The sample handling module of claim 5, wherein the user interface subsystem further comprises one or more output chutes, each of which is configured to output individual sample containers.

10. The sample handling module of claim 9, wherein the user interface subsystem further comprises a display having a graphical user interface (GUI) through which the one or more compartments and the one or more output chutes may be selected for the output of positive sample containers.

11. The sample handling module of claim 5, wherein the user interface subsystem further comprises a sliding door configured to prevent a user from loading one or more untested sample containers into at least one of the compartments while the at least one compartment is being loaded with one or more positive sample containers by the robotic subsystem.

12. The sample handling module of claim 5, wherein at least one of the compartments comprises a scale, and wherein the sample handling module further comprises one or more processors configured to determine whether the untested sample containers are overfilled or underfilled based weight measurements received from the scale.

13. The sample handling module of claim 5, wherein at least one of the compartments comprises a scale, and wherein the sample handling module further comprises one or more processors configured to:
receive from the scale a first measured weight of a plurality of untested sample containers in the at least one compartment;
control the robotic subsystem to transfer one of the untested sample containers from the at least one compartment to the imaging subsystem;
receive from the scale a second measured weight of the untested sample containers in the at least one compartment without the one untested sample container;
determine a difference between the first and second measured weights; and
store the difference as a weight of the one untested sample container in memory.

14. The sample handling module of claim 1, wherein the user interface subsystem further comprises:
a reader configured to scan identifiers on sample containers; and
a display configured to provide information for sample containers scanned by the reader.

15. The sample handling module of claim 1, wherein the user interface subsystem further comprises a reader configured to scan an identifier on a user identification card to initiate an automatic login or automatically adjust one or more system settings.

16. The sample handling module of claim 1, wherein each of the plurality of untested sample containers is received at the user interface subsystem in an upright position, and wherein each of the plurality of untested sample containers is transferred from the imaging subsystem to the incubation module in a horizontal position.

17. The sample handling module of claim 16, wherein each of the plurality of positive sample containers is transferred from the incubation module to the imaging subsystem in a horizontal position, and wherein each of the plurality of positive sample containers is transferred from the imaging subsystem to the user interface subsystem in an upright position.

18. The sample handling module of claim 17, wherein the imaging subsystem comprises:
a camera for scanning the sample containers or capturing one or more images of the sample containers;
one or more light sources for illuminating the sample containers;
a chute configured to reorient sample containers from an upright position to a horizontal position; and
a flip station configured to reorient sample containers from a horizontal position to an upright position.

19. The sample handling module of claim 1, wherein the imaging subsystem is further configured to capture one or more images of the sample containers, and wherein the sample handling module further comprises one or more processors configured to determine whether the sample containers are overfilled or underfilled based on the captured images.

20. The sample handling module of claim 1, wherein the imaging subsystem is further configured to capture one or more images of the sample containers, and wherein the sample handling module further comprises one or more processors configured to:
identify a position of a fill line on a sample container in the one or more images;
identifying a position of a reference surface on the sample container in the one or more images;
determine a distance between the fill line and the reference surface;
determine a correction factor based on a comparison between the determined distance and a predetermined distance;
adjust a predetermined tare weight with the correction factor; and
determine whether the sample container is overfilled or underfilled based on a comparison between the adjusted predetermined tare weight and a measured weight of the sample container obtained with a scale.

21. The sample handling module of claim 20, wherein the imaging subsystem comprises a chute configured to reorient sample containers from an upright position to a horizontal position, wherein the scale is coupled to the chute of the imaging subsystem, and wherein the measured weight is obtained while the sample container is positioned in the chute.

22. The sample handling module of claim 1, wherein the waste management subsystem comprises a waste receptacle and one or more chutes through which the robotic subsystem can transfer the plurality of negative sample containers into the waste receptacle.

23. The sample handling module of claim 1, wherein the waste management subsystem comprises a load cell configured to (a) detect whether a waste receptacle if full, (b) detect whether a waste receptacle is positioned on a base, or (c) detect the addition of a sample container to a waste receptacle.

24-27. (canceled)

28. A robotic system comprising:
a gripper assembly configured to grab and release sample containers;
an r-axis robot configured to move the gripper assembly forwards and backwards;
a theta-axis robot configured to simultaneously rotate the r-axis robot and the gripper assembly; and
a z-axis robot configured to simultaneously move the theta-axis robot, the r-axis robot, and the gripper assembly upwards and downwards.

29. The robotic system of claim 28, wherein the gripper assembly comprises:
a motor; and
two grippers, wherein the motor is configured to move the two grippers closer together to grasp a sample container and to move the two grippers farther apart to release a sample container, and wherein each gripper comprises:
a curved body configured to grasp a bottom end of a sample container in a horizontal position; and
a curved recess in the curved body that is configured to grasp a neck of a sample container in an upright position.

30. The robotic system of claim 29, wherein the curved body is configured to grasp a bottom end of a blood culture bottle in a horizontal position, and wherein the curved recess is configured to grasp a neck of a blood culture bottle in an upright position.

31. The robotic system of claim 28, wherein the gripper assembly comprises:
a motor; and
two grippers, wherein the motor is configured to move the two grippers closer together to grasp a sample container and to move the two grippers farther apart to release a sample container, and wherein each gripper comprises:
a plurality of fingers configured to grasp a bottom end of a sample container in a horizontal position; and
a curved recess in a body of the gripper that is configured to grasp a neck of a sample container in an upright position.

32. The robotic system of claim 31, wherein the fingers are configured to grasp a bottom end of a blood culture bottle in a horizontal position, and wherein the curved recess is configured to grasp a neck of a blood culture bottle in an upright position.

33. The robotic system of claim 28, wherein the r-axis robot comprises:
a first arm coupled to the theta-axis robot;
a second arm coupled to the gripper assembly, wherein the second arm is slidingly engaged with the first arm and configured to move forwards and backwards;
a plurality of idler pulleys;
a motor coupled to the first arm and positioned between at least two of the idler pulleys;
a drive pulley coupled to a shaft of the motor;
a belt contacting each of the idler pulleys and the drive pulley; and
a clamp coupled to the belt and the second arm.

34. The robotic system of claim 33, wherein the r-axis robot further comprises a belt tensioner configured to apply tension to the belt.

35. The robotic system of claim 34, wherein the belt tensioner comprises
an idler pulley contacting the belt;
an arm rotatably coupled to the idler pulley and a coupling; and
a screw configured to apply force to the arm when tightened, wherein the force from the screw causes (a) the arm to rotate about an axis that extends through the coupling and (b) the idler pulley to apply additional tension to the belt.

36. The robotic system of claim 28, wherein the theta-axis robot comprises:
a platform coupled to the z-axis robot;
an idler pully coupled to the r-axis robot;
a motor coupled to the platform;
a drive pulley coupled to a shaft of the motor; and
a belt contacting the idler pulley and the drive pulley,
wherein rotation of the drive pully by the motor causes the idler pully, the r-axis robot, and the gripper assembly to simultaneously rotate.

37. The robotic system of claim 28, wherein the z-axis robot comprises a rail slidingly engaged with the theta-axis robot.

38. The robotic system of claim 37, wherein the z-axis robot further comprises a counterweight system comprising:
a counterweight;
one or more pulleys; and
at least one cable that contacts the one or more pulleys and is coupled to both the counterweight and the theta-axis robot.

39-54. (canceled)
